SEQUENCE LISTING (41 sequences)

SEQ ID NO: 1 - protein, 10 aa, synthetic construct
  VARIANT 3: note = N or T
  VARIANT 5: note = E or G
  VARIANT 6: note = R or A
  VARIANT 10: note = S or V
GFXFXXYDMX

SEQ ID NO: 2 - protein, 10 aa, synthetic construct
RLNSFGRSTY

SEQ ID NO: 3 - protein, 25 aa, synthetic construct
  VARIANT 2: note = V or A
  VARIANT 10: note = G or A
  VARIANT 23: note = E or A
EXQLVESGGXLVQPGGSLRLSCXAS

SEQ ID NO: 4 - protein, 14 aa, synthetic construct
  VARIANT 4: note = Q or H
  VARIANT 6: note = P or T
  VARIANT 13: note = V or I
WVRXAXGKGPEWXS

SEQ ID NO: 5 - protein, 38 aa, synthetic construct
  VARIANT 2: note = L or A
  VARIANT 3: note = D or S
  VARIANT 7: note = G or D
  VARIANT 11: note = V or I
  VARIANT 15: note = N or T
  VARIANT 17: note = Q or K
  VARIANT 18: note = N or S
  VARIANT 19: note = M or T
  VARIANT 20: note = L or F
  VARIANT 32: note = T or A
  VARIANT 33: note = A or G
YXXSVKXRFTXSRDXAXXXXYLQMNNLKPEDXXVYYCA

SEQ ID NO: 6 - protein, 11 aa, synthetic construct
  VARIANT 6: note = Q or L
WGQGTXVTVSS

SEQ ID NO: 7 - protein, 10 aa, synthetic construct
GFNFERYDMS

SEQ ID NO: 8 - protein, 10 aa, synthetic construct
GFTFGAYDMV

SEQ ID NO: 9 - protein, 10 aa, synthetic construct
RLNSFGRSTY

SEQ ID NO: 10 - protein, 10 aa, synthetic construct
FISNGGEEVS

SEQ ID NO: 11 - protein, 19 aa, synthetic construct
RSTDVSPGLSSWWTYEYDV

SEQ ID NO: 12 - protein, 25 aa, synthetic construct
EVQLVESGGGLVQPGGSLRLSCEAS

SEQ ID NO: 13 - protein, 25 aa, synthetic construct
EAQLVESGGGLVQPGGSLRLSCEAS

SEQ ID NO: 14 - protein, 25 aa, synthetic construct
EVQLVESGGALVQPGGSLRLSCAAS

SEQ ID NO: 15 - protein, 14 aa, synthetic construct
WVRQAPGKGPEWVS

SEQ ID NO: 16 - protein, 14 aa, synthetic construct
WVRHATGKGPEWIS

SEQ ID NO: 17 - protein, 38 aa, synthetic construct
YLDSVKGRFTVSRDNAQNMLYLQMNNLKPEDTAVYYCA

SEQ ID NO: 18 - protein, 38 aa, synthetic construct
YLDSVKGRFTVSRDTAKSTFYLQMNNLKPEDAGVYYCA

SEQ ID NO: 19 - protein, 38 aa, synthetic construct
YASSVKDRFTISRDNAQNMLYLQMNNLKPEDTAVYYCA

SEQ ID NO: 20 - protein, 11 aa, synthetic construct
WGQGTQVTVSS

SEQ ID NO: 21 - protein, 11 aa, synthetic construct
WGQGTLVTVSS

SEQ ID NO: 22 - protein, 127 aa, synthetic construct
EVQLVESGGGLVQPGGSLRLSCEASGFNFERYDMSWVRQAPGKGPEWVSRLNSFGRSTYYLDSVKGRFTVSRDNAQNMLYLQMNNLKPEDTAVYYCARSTDVSPGLSSWWTYEYDVWGQGTQVTVSS

SEQ ID NO: 23 - DNA, 381 bp, synthetic construct
  source 1..381: mol_type = other DNA
gaggtccagctggtggagtctgggggaggcttggtgcagcctgggggttctctgagactctcctgtgaagcctctggattcaattttgaaagatatgatatgagttgggtccgccaggctccaggaaaggggcccgagtgggtgtcacgtctgaatagttttggacggagcacatattacttagattctgtgaagggccgattcaccgtttccagagacaacgcccagaacatgctatatctgcaaatgaacaacctgaaacctgaggacacggccgtgtattactgtgcaaggtctacggacgtctcacccgggctaagtagttggtggacatatgagtatgacgtttggggccaggggacccaggtcaccgtctccagc

SEQ ID NO: 24 - protein, 127 aa, synthetic construct
EAQLVESGGGLVQPGGSLRLSCEASGFNFERYDMSWVRQAPGKGPEWVSRLNSFGRSTYYLDSVKGRFTVSRDTAKSTFYLQMNNLKPEDAGVYYCARSTDVSPGLSSWWTYEYDVWGQGTQVTVSS

SEQ ID NO: 25 - DNA, 381 bp, synthetic construct
  source 1..381: mol_type = other DNA
gaggcccagctggtggagtctgggggaggcttggtgcagcctgggggttctctgagactctcctgtgaagcctctggattcaattttgaaagatatgatatgagttgggtccgccaggctccaggaaaggggcccgagtgggtgtcacgtctgaatagttttggacggagcacatattacttagattctgtgaagggccgattcaccgtttccagagacacggccaagagtacgttttatctgcaaatgaacaacctgaaacctgaggatgcgggcgtgtattactgtgcaaggtctacggacgtctcacccgggctaagtagttggtggacatatgagtatgacgtttggggccaggggacccaggtcaccgtctccagc

SEQ ID NO: 26 - protein, 127 aa, synthetic construct
EVQLVESGGALVQPGGSLRLSCAASGFTFGAYDMVWVRHATGKGPEWISFISNGGEEVSYASSVKDRFTISRDNAQNMLYLQMNNLKPEDTAVYYCARSTDVSPGLSSWWTYEYDVWGQGTLVTVSS

SEQ ID NO: 27 - DNA, 381 bp, synthetic construct
  source 1..381: mol_type = other DNA
gaggttcagctggtggagtctgggggagccttggtgcagcctgggggttctctgagactctcctgtgcagcctctggattcaccttcggagcctatgacatggtctgggtccgccacgctactggaaaggggcccgaatggatctcattcatctctaatggcggtgaagaggtatcgtacgcgagctccgtgaaggaccgattcaccatctccagagacaatgcccagaacatgctatatctgcaaatgaacaacctgaaacctgaggacacggccgtgtattactgtgcaaggtctacggacgtctcacccgggctaagtagttggtggacatatgagtatgacgtttggggccaggggaccctggtcaccgtctccagc

SEQ ID NO: 28 - protein, 395 aa, synthetic construct
METDTLLLWVLLLLAAQPAMAAGSEVQLVESGGGLVQPGGSLRLSCEASGFNFERYDMSWVRQAPGKGPEWVSRLNSFGRSTYYLDSVKGRFTVSRDNAQNMLYLQMNNLKPEDTAVYYCARSTDVSPGLSSWWTYEYDVWGQGTQVTVSSGPGGQGTGPGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - DNA, 1188 bp, synthetic construct
  source 1..1188: mol_type = other DNA
atggagactgacaccctgcttctttgggtgcttttgcttctcgccgcacaacctgctatggccgctggatccgaagttcaacttgtagaaagtgggggaggtctggtacaaccaggcggttctctgagactctcctgcgaggcttctgggtttaacttcgaacgctatgacatgagttgggtgcggcaggcaccggggaaaggaccggaatgggtctcccggctgaatagctttgggcgcagcacatattaccttgatagcgttaagggacgcttcacggtatcacgagataatgctcaaaacatgttgtatctccagatgaataatctgaaaccggaagacacagcagtgtactattgcgcaagaagcacagacgtttctcctggtctctcatcttggtggacctatgaatatgatgtttggggccagggcacgcaagttacggtcagtagtgggccgggcggccagggtaccggacccggtgggtctgaacctaaatcatctgataaaacgcacacatgccccccatgtcctgcgccagagctgctgggcggtccgtccgttttcctttttcctcctaagccgaaagatacgttgatgatttcaaggacgcctgaggtaacttgcgtggtcgtagatgtaagccatgaagacccggaggtcaagttcaactggtacgtagacggagtcgaagtacataacgcgaagacgaagccacgcgaggaacagtacaacagcacgtatagggtagtcagcgtactgaccgtcttgcatcaggattggttgaacggtaaggagtacaagtgtaaagtgtccaataaggcccttcccgcgccgattgaaaagacgattagcaaagccaaaggccaaccgcgagagccacaggtttacaccctgccgccgtcccgcgatgaacttaccaagaaccaggtcagtctgacttgccttgtaaaaggtttctacccatctgacatagccgtggagtgggaatccaatgggcaaccagagaataactataagactactcctccggtgcttgattctgacgggtcttttttcttgtatagcaaactcacggttgataaatctaggtggcaacagggaaatgtatttagttgttctgtaatgcacgaagcccttcacaatcactacacccaaaagagtctctctctctctcccggcaagtga

SEQ ID NO: 30 - protein, 1390 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 31 - protein, 1390 aa, Homo sapiens; identical to SEQ ID NO: 30

SEQ ID NO: 32 - protein, 232 aa, Homo sapiens
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 5 aa, synthetic construct
GSGGS

SEQ ID NO: 34 - protein, 4 aa, synthetic construct
GGGS

SEQ ID NO: 35 - protein, 5 aa, synthetic construct
GGGGS

SEQ ID NO: 36 - protein, 9 aa, synthetic construct
GGGGSGGGS

SEQ ID NO: 37 - protein, 15 aa, synthetic construct
GGGGSGGGGSGGGGS

SEQ ID NO: 38 - protein, 5 aa, synthetic construct
GPGGP

SEQ ID NO: 39 - protein, 36 aa, synthetic construct
AALVGPGGQGGGGSGGGGSGGGGSGGGGSGGGGSMA

SEQ ID NO: 40 - protein, 15 aa, synthetic construct
EPKSSDKTHTSPPSP

SEQ ID NO: 41 - protein, 12 aa, synthetic construct
GPGGQGTGPGGS

(19) United States
(12) Patent Application Publication
(10) Pub. No.: US 2025/0152759 A1
(43) Pub. Date: May 15, 2025

(54) SINGLE-DOMAIN ANTIBODIES AND VARIANTS THEREOF AGAINST MET

(71) Applicant: Wisconsin Alumni Research Foundation, Madison, WI (US)

(72) Inventors: Andrew Baschnagel, Verona, WI (US); Randall Kimple, Madison, WI (US); Reinier Hernandez, Madison, WI (US); Aaron LeBeau, Middleton, WI (US)

(73) Assignee: Wisconsin Alumni Research Foundation, Madison, WI (US)

(21) Appl. No.: 18/947,271

(22) Filed: Nov. 14, 2024

Related U.S. Application Data
(60) Provisional application No. 63/598,830, filed on Nov. 14, 2023.

(51) Int. Cl. A61K 51/10; C07K 16/22; C07K 16/28

(52) U.S. Cl. CPC A61K 51/1027; C07K 16/22; C07K 16/28; A61K 2121/00; A61K 2123/00; C07K 2317/31; C07K 2317/52; C07K 2317/565; C07K 2317/569; C07K 2319/00

(57) ABSTRACT

The present application provides constructs comprising a single-domain antibody (sdAb) moiety that specifically recognizes mesenchymal epithelial transition factor receptor (MET). Also provided are methods of making and using these constructs.

### CROSS-REFERENCE TO RELATED APPLICATIONS

Priority is hereby claimed to U.S. Provisional Application 63/598,830, filed Nov. 14, 2023, which is incorporated herein by reference in its entirety.

### SEQUENCE LISTING

The instant application contains a Sequence Listing which has been submitted in XML format and is hereby incorporated by reference in its entirety. The XML copy, created on Sep. 17, 2024, is named USPTO-09824565-P230173US02-SEQ_LIST.xml and is 50,754 bytes in size.

### FIELD OF THE INVENTION

The present invention relates to constructs comprising a single-domain antibody (sdAb) moiety that specifically recognizes mesenchymal epithelial transition factor receptor (MET), and methods of making and using thereof.

### BACKGROUND

Non-small cell lung cancer (NSCLC) comprises approximately 85% of all lung cancer cases and is the leading cause of cancer death in the United States. Among patients with NSCLC, 3-4% have a mutated mesenchymal epithelial transition (MET) gene, and 3-6% have an amplified form of this gene (Digumarthy et al. 2019; Okuda et al. 2008). MET, also known as c-MET or hepatocyte growth factor receptor (HGFR), is a transmembrane receptor tyrosine kinase that plays a key role in cell proliferation, embryogenesis, tissue development, and wound healing (Birchmeier et al. 1998, Reungwetwattana et al. 2017, Birchmeier et al. 2003, Trusolino et al. 2010). The most common MET mutation occurs in exon 14, which leads to a truncated receptor lacking the Y1003 c-Cb1 binding site, resulting in decreased MET protein ubiquitination and increased expression of the MET protein (Cancer Genome Atlas Research Network 2014, Peschard et al. 2001, Rolle et al. 2016). Both MET exon skipping (METex14) mutations and MET amplifications can be targeted with therapy. Currently, crizotinib, capmatinib, and tepotinib are the 3 tyrosine kinase inhibitors that are FDA-approved for treating METex14 NSCLC (Desai et al. 2022, Hong et al. 2021). While these therapies are initially effective, resistance inevitably arises and new more effective therapies are needed. Since both METex14 mutations and MET amplifications lead to high surface expression and receptor activation, the MET receptor represents an ideal target for antibody-based imaging and therapy. A number of monoclonal antibody therapies directed against MET exists that are in various preclinical and clinical stages of development in NSCLC. Thus far, however, results using conventional mono- and bi-specific antibodies against MET have been disappointing with little therapeutic benefit in the clinic (Spigel et al. 2017, Camidge et al. 2022, Scagliotti et al. 2020, Camidge et al. 2021, Waqar et al. 2021).

None of the previous MET antibody studies in NSCLC utilized camelid-derived nanobodies. VHH domains from camelid heavy-chain antibodies have the potential to be a more effective imaging and therapeutic agent than conventional monoclonal antibodies (FIG. 1A) (Muyldermans et al. 2013). These VVHs are characterized by their small size (˜15 kDa, 4 nm by 2.5 nm), high solubility, stability, specificity and affinity, and ease of cloning (Van Audenhove et al. 2016). The complementary determining regions (CDRs) of camelid VHHs can be more than 20 amino acids long, more than twice the length of a human CDR, yet the VHH is still only a fraction of the size of a human binding domain (Muyldermans et al. 2021). This is because the camelid VHH only has 3 heavy chain CDRs instead of an additional 3 light chain CDRs that a human antibody has (Konning et al. 2017). These unique traits provide VHHs with a unique architecture and molecular dexterity, allowing them to bind epitopes inaccessible to conventional antibodies (cryptic epitopes) with unparalleled affinity.

There is a critical unmet need for effective antibody-based therapies that can target MET on the surface of transformed cells and for treating other conditions.

### SUMMARY OF THE INVENTION

On aspect of the invention is directed to isolated anti-MET constructs comprising a single-domain antibody (sdAb) moiety specifically recognizing MET.

In some versions, the sdAb moiety comprises a CDR1, a CDR2, and a CDR3.

In some versions, the sdAb moiety comprises:
a CDR1 comprising the amino acid sequence of GFX¹FX²X³YDMX⁴ (SEQ ID NO:1), wherein:
   X¹ is N or T;
   X² is E or G;
   X³ is R or A; and
   X⁴ is S or V,
      or a variant thereof comprising up to about 3 (such as about any of 1, 2, or 3) amino acid substitutions;
a CDR2 comprising the amino acid sequence of X⁵X⁶X⁷X⁸X⁹GX¹⁰X¹¹X¹²X¹³, wherein:
   X⁵ is R or F;
   X⁶ is L or I;
   X⁷ is N or S;
   X⁸ is S or N;
   X⁹ is F or G;
   X¹⁰ is R or E;
   X¹¹ is S or E;
   X¹² is T or V; and
   X¹³ is Y or S,
      or a variant thereof comprising up to about 3 (such as about any of 1, 2, or 3) amino acid substitutions; and/or
a CDR3 comprising the amino acid sequence of RSTDVSPGLSSWWTYEYDV (SEQ ID NO: 11), or a variant thereof comprising up to about 3 (such as about any of 1, 2, or 3) amino acid substitutions.

In some versions, the sdAb moiety comprises:
a CDR1 comprising the amino acid sequence of any one of GENFERYDMS (SEQ ID NO: 7) and GFTFGAYDMV (SEQ ID NO:8), or a variant thereof comprising up to about 3 (such as about any of 1, 2, or 3) amino acid substitutions;
a CDR2 comprising the amino acid sequence of any one of RLNSFGRSTY (SEQ ID NO: 9) and FISNGGEEVS (SEQ ID NO:10), or a variant thereof comprising up to about 3 (such as about any of 1, 2, or 3) amino acid substitutions; and/or
a CDR3 comprising the amino acid sequence of RSTDVSPGLSSWWTYEYDV (SEQ ID NO: 11), or a variant thereof comprising up to about 3 (such as about any of 1, 2, or 3) amino acid substitutions.

In some versions, the sdAb moiety comprises:
a CDR1 comprising the amino acid sequence of any one of GENFERYDMS (SEQ ID NO: 7) and GFTFGAYDMV (SEQ ID NO:8);
a CDR2 comprising the amino acid sequence of any one of RLNSFGRSTY (SEQ ID NO: 9) and FISNGGEEVS (SEQ ID NO:10); and/or
a CDR3 comprising the amino acid sequence of RSTDVSPGLSSWWTYEYDV (SEQ ID NO: 11).

In some versions, the sdAb moiety comprises:
a CDR1 comprising the amino acid sequence of GENFERYDMS (SEQ ID NO:7) or a variant thereof comprising up to about 3 (such as about any of 1, 2, or 3) amino acid substitutions, a CDR2 comprising the amino acid sequence of RLNSFGRSTY (SEQ ID NO:9) or a variant thereof comprising up to about 3 (such as about any of 1, 2, or 3) amino acid substitutions, and CDR3 the amino a comprising acid sequence of RSTDVSPGLSSWWTYEYDV (SEQ ID NO:11) or a variant thereof comprising up to about 3 (such as about any of 1, 2, or 3) amino acid substitutions; or
a CDR1 comprising the amino acid sequence of GFTFGAYDMV (SEQ ID NO:8) or a variant thereof comprising up to about 3 (such as about any of 1, 2, or 3) amino acid substitutions, a CDR2 comprising the amino acid sequence of FISNGGEEVS (SEQ ID NO:10) or a variant thereof comprising up to about 3 (such as about any of 1, 2, or 3) amino acid substitutions, and a CDR3 comprising the amino acid sequence of RSTDVSPGLSSWWTYEYDV (SEQ ID NO:11) or a variant thereof comprising up to about 3 (such as about any of 1, 2, or 3) amino acid substitutions.

In some versions, the sdAb moiety comprises:
a CDR1 comprising the amino acid sequence of GENFERYDMS (SEQ ID NO:7), a CDR2 comprising the amino acid sequence of RLNSFGRSTY (SEQ ID NO:9), and a CDR3 comprising the amino acid sequence of RSTDVSPGLSSWWTYEYDV (SEQ ID NO:11); or
a CDR1 comprising the amino acid sequence of GFTFGAYDMV (SEQ ID NO:8), a CDR2 comprising the amino acid sequence of FISNGGEEVS (SEQ ID NO:10), and a CDR3 comprising the amino acid sequence of RSTDVSPGLSSWWTYEYDV (SEQ ID NO:11).

In some versions, the sdAb moiety comprises:
an FR1 comprising the amino acid sequence of EX¹⁴QLVESGGX¹⁵LVQPGGSLRLSCX¹⁶AS (SEQ ID NO:3), wherein:
   X¹⁴ is V or A;
   X¹⁵ is G or A; and
   X¹⁶ is E or A,
      or a variant thereof comprising up to about 3 (such as about any of 1, 2, or 3) amino acid substitutions;
an FR2 comprising the amino acid sequence of WVRX¹⁷AX¹⁸GKGPEWX¹⁹S (SEQ ID NO: 4), wherein:
   X¹⁷ is Q or H;
   X¹⁸ is P or T; and
   X¹⁹ is V or I,
      or a variant thereof comprising up to about 3 (such as about any of 1, 2, or 3) amino acid substitutions;
an FR3 comprising the amino acid sequence of YX²⁰X²¹SVKX²²RFTX²³SRDX²⁴AX²⁵X²⁶X²⁷X²⁸YLQMNNLKPEDX²⁹X³⁰VYYCA (SEQ ID NO: 5), wherein:
   X²⁰ is L or A;
   X²¹ is D or S;
   X²² is G or D;
   X²³ is V or I;
   X²⁴ is N or T;
   X²⁵ is Q or K;
   X²⁶ is N or S;
   X²⁷ is M or T;
   X²⁸ is L or F;
   X²⁹ is T or A; and
   X³⁰ is A or G,
      or a variant thereof comprising up to about 3 (such as about any of 1, 2, or 3) amino acid substitutions; and/or
an FR4 comprising the amino acid sequence of WGQGTX³¹VTVSS (SEQ ID NO:6), wherein X³¹ is Q or L, or a variant thereof comprising up to about 3 (such as about any of 1, 2, or 3) amino acid substitutions.

In some versions, the sdAb moiety comprises any one of the following:
a VHH domain comprising the amino acid sequence of SEQ ID NO:22, or a variant thereof comprising a CDR1 comprising the amino acid sequence of GENFERYDMS (SEQ ID NO: 7), a CDR2 comprising the amino acid sequence of RLNSFGRSTY (SEQ ID NO:9), and a CDR3 comprising the amino acid sequence of RSTDVSPGLSSWWTYEYDV (SEQ ID NO: 11) and having at least about 90% or at least about 95% sequence identity to SEQ ID NO: 22;
a VHH domain comprising the amino acid sequence of SEQ ID NO:24, or a variant thereof comprising a CDR1 comprising the amino acid sequence of GENFERYDMS (SEQ ID NO: 7), a CDR2 comprising the amino acid sequence of RLNSFGRSTY (SEQ ID NO:9), and a CDR3 comprising the amino acid sequence of RSTDVSPGLSSWWTYEYDV (SEQ ID NO: 11) and having at least about 90% or at least about 95% sequence identity to SEQ ID NO: 24; or
a VHH domain comprising the amino acid sequence of SEQ ID NO:26, or a variant thereof comprising a CDR1 comprising the amino acid sequence of GFTFGAYDMV (SEQ ID NO: 8), a CDR2 comprising the amino acid sequence of FISNGGEEVS (SEQ ID NO:10), and a CDR3 comprising the amino acid sequence of RSTDVSPGLSSWWTYEYDV (SEQ ID NO: 11) and having at least about 90% or at least about 95% sequence identity to SEQ ID NO: 26.

In some versions, the sdAb moiety specifically recognizing MET is camelid, chimeric, partially humanized, or fully humanized.

In some versions, the sdAb moiety specifically recognizing MET is fused to a human IgG1 Fc.

In some versions, the isolated anti-MET construct is a heavy chain-only antibody.

In some versions, the isolated anti-MET construct is fused to a second antibody moiety. In some versions, the isolated anti-MET construct is fused to an anti-EGFR antibody moiety.

In some versions, the isolated anti-MET construct is labeled. In some versions, the isolated anti-MET construct is radiolabeled.

In some versions, the sdAb moiety is conjugated to a cytotoxic agent.

Another aspect of the invention is directed to pharmaceutical compositions. In some versions, the pharmaceutical compositions comprise an isolated anti-MET construct of the invention and a pharmaceutically acceptable carrier.

Another aspect of the invention is directed to methods of treating a MET-related condition in an individual in need thereof. In some versions, the methods comprise administering to the individual a therapeutically effective amount of an isolated anti-MET construct of the invention.

In some versions, the MET-related disease is cancer. In some versions, the cancer is selected from the group consisting of lung cancer and head and neck cancer.

In some versions, the methods further comprise screening the individual, wherein the screening comprises administering a screening amount of an isolated anti-MET construct of the invention to the individual and imaging the individual for presence of the isolated anti-MET construct in the individual.

Another aspect of the invention is directed to methods of screening an individual. In some versions, the methods comprise administering a screening amount of an isolated anti-MET construct of the invention to the individual and imaging the individual for presence of the isolated anti-MET construct in the individual.

The objects and advantages of the invention will appear more fully from the following detailed description of the preferred embodiment of the invention made in conjunction with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A. Schematic with the average size of an IgG antibody, camelid heavy chain antibody, and VHH.

FIG. 1B. Schematic of exemplary generation and uses of the anti-MET VHHs of the invention.

FIGS. 2A-2B. Discovery of potential anti-MET VHHs from a camelid antibody phage display library. FIG. 2A. Screening of VHH clones with c-MET specificity by ELISA. 384 unpurified VHH clones in culture supernatant were added to wells coated with c-MET protein. VHH binding was detected using a peroxidase conjugated anti-HA-tag monoclonal antibody and Turbo TMB reagent. 12 clones with high 420 nm Absorbance values (greater than 0.9) were identified. FIG. 2B. Dilution ELISA w VHH clones with high affinity to c-MET. 12 unpurified VHH were serially diluted from 1 to 0.05 relative concentration and added to human c-MET protein coated plates. Two clones showed saturating ELISA signals.

FIGS. 3A-3D. Characterization and selection of 1E7 VHH among 3 VHHs with high MET binding. FIG. 3A. Specificity of 1E7, 1A2, and 2G9 VHHs for human (h), rhesus macaque (mk), and murine (ms) MET was determined by ELISA. Unpurified VHH clones were serially diluted from 1 to 0.00025 and incubated on plates that were coated with the target protein. 1E7 showed saturating ELISA signals on human MET and minimal binding to rhesus macaque and murine MET. 1A2 and 2G9 did not show clear specificity. FIGS. 3B and 3C. Biolayer interferometry was used to calculate and compare 1E7 (FIG. 3B), 1A2 (FIG. 3C), and 2G9 (FIG. 3D) binding affinities to human MET. 1E7 demonstrated the highest binding affinity, whereas 1A2 and 2G9 showed weaker binding affinities to human MET.

FIGS. 4A-4C. In vitro analysis of 1E7-Fc illustrating specificity to c-MET-positive cell lines. FIG. 4A. Confirmation of 1E7-Fc specificity for MET using BLI. 1E7-Fc demonstrated binding only to MET (black line) and minimal to no binding to other surface proteins. FIG. 4B. 1E7-Fc selectivity for MET expressing cells was assessed by flow cytometry. Cells were stained with 1E7-Fc at 1 μg/mL for 1 hour (green) and then with a human 1gG Fc secondary antibody, PE, at 5 μg/mL for 1 hour. Unstained cells (grey) were used as negative controls.

FIG. 4C. Cellular internalization of 1E7-Fc was visualized by immunofluorescent confocal microscopy. Live cells were stained with fluorescent cell plasma membrane dye (green), placed in a TOKEI biochamber, treated with 1 mM of VHH-Fc for 10 minutes (red), and imaged every minute for 2 hours by Nikon Spinning Disk Microscope at 40x. Images at beginning and end of the sequence were collected and colorized through Nikon NIS-Elements.

FIGS. 5A and 5B. 1E7-Fc does not inhibit cell growth or block MET signaling pathways. FIG. 5A. Proliferation assay shows that 1E7-Fc does not impact cell survival. Effect of 1E7-Fc on cell viability in vitro was assessed for indicated cell lines. Viability was assessed using XXX assay 72 h after treatment with indicated concentrations of VHH-Fc. Points mean; bar SEM (n=6). FIG. 5B. Western blots were performed to assess MET, p-MET (Tyr1234/1235), AKT, p-AKT (Ser473), ERK, p-ERK (Thr202/Tyr204), and GAPDH in cells treated with 1E7-Fc. Cells were treated respective drugs for 4 h and Capmatinib (5 nM) was used as a positive control.

FIGS. 6A-6D. 1E7-Fc targets and accumulates in MET-expressing xenografts. FIGS. 6A and 6B. [89Zr]Zr-1E7-Fc can detect MET-positive lung cancer xenografts by PET/CT imaging. Representative PET/CT images of mice bearing EBC-1 (FIG. 6A) and UW-Lung 21 (FIG. 6B) subcutaneous xenografts. Mice (n=3 per cell line) received around 5.5 MBq [89Zr]Zr-1E7-Fc via tail vein and imaged at the labeled time points. FIG. 6C. Representative 3D PET/CT images of [89Zr]Zr-1E7-Fc in mice bearing EBC-1 and UW-Lung 21 subcutaneous xenografts at 48 h. FIG. 6D. In vivo validation of MET expression on tumor xenografts. Representative images of MET IHC staining in EBC-1 and UW-Lung 21 xenografts. Scale bar 50 μm.

FIGS. 7A-7D. Confirmation of [89Zr]Zr-1E7-Fc selectivity and stability in vivo. FIG. 7A. Quantitative analysis of the same subcutaneous xenografts as FIGS. 6A-6D. Regions of interest (ROIs) were drawn to obtain values for the heart and tumor for the indicated time points. The inverse relationship between the organs suggests all radiolabeled 1E7-Fc localized in the MET-positive xenograft over time. FIG. 7B. Biodistribution of [89Zr]Zr1E7-Fc in organs at 24 h. Organs and tissues are harvested at 24 h, and the radioactive counts were measured for each one. There is a significantly higher uptake of [89Zr]Zr-1E7-Fc in the tumor relative to muscle. Values are the mean (n=3)±SEM. FIG. 7C. Biodistribution of [89Zr]Zr-1E7-Fc at 24 h in the tumor is significantly higher than in the bone and muscle. Values are the mean (n=3)±SEM. FIG. 7D. In vivo stability of 1E7-Fc is around 3 days. 1E7-Fc was injected into mice via the tail vein, and blood was collected at 8 time points for sera collection. The amount of 1E7-Fc remaining in the sera that binds to human MET was measured with a dilution ELISA. Data are the mean (n=3)±SEM.

FIGS. 8A-8D. MET expression is highly prevalent in HNSCC and correlates with worse prognosis. FIG. 8A. Distribution of MET protein expression of the 203 patient samples based on tissue microarray (TMA) analysis reveals majority of HNSCC had MET expression. FIG. 8B. Representative TMA immunostaining (top) and immunofluorescent (bottom) images. Staining allowed for algorithm-based categorization from individual signals (red=MET, blue=DAPI, green=pan-cytokeratin). FIGS. 8C and 8D. Kaplan-Meier curves from patients with HNSCC queried from TCGA database demonstrating that while MET expression did not serve as a significant prognostic marker in patients with HNSCC who were HPV-positive (FIG. 8C), high MET expression correlates with worse PFS in patients who were HPV-negative (FIG. 8D).

FIGS. 9A-9H. Camelid Antibody 1E7-Fc selectively binds to high MET expressing HNSCC cells. FIGS. 9A and 9B. Western blotting (FIG. 9A) and xenograft IHC analysis (FIG. 9B) was performed to assess MET and phospho-MET (p-Met; Tyr1234/1235) in cell lines UD-SCC2, UM-SCC47, UPCI-SCC90, UM-SCC1, Detroit 562, and T-47D. FIG. 9C-9H. Flow Cytometry demonstrating binding affinity by comparing fluorescent intensity of cells exposed to 100 nM 1E7-Fc (pink) and controls (blue).

FIG. 10. Camelid Antibody 1E7-Fc is internalized when bound to MET expressing Detroit 562 cell line. Immunofluorescent live cell imaging of binding and internalization of 1E7-Fc (red) on Detroit 562 and T-47D relative to cell membrane (green) and nucleus (blue). Top row presents merged signals from fluorescent channels and bottom row presents isolated channel from far-red camelid fluorescent.

FIGS. 11A-11D and 11B. Camelid Antibody does inhibit cell proliferation or cause cytotoxicity. FIGS. 11A-11C. Sensitivity to 1E7-Fc was measured by proliferation assay. FIG. 11D. Western blotting was used to assess MET, phospho-MET (p-Met; Tyr1234/1235), AKT, phospho-AKT (p-AKT; Ser473), MAPK/ERK, phospho-MAPK/ERK (p-MAPK/ERK; Thr202/Tyr204) in Detroit 562 and T-47D. Cells were pretreated with 1E7-Fc (10 nm or 100 nM) or capmatinib (5 nM) for 4, 24, or 48 hours and collected after at indicated time points.

FIGS. 12A-12C. 1E7-Fc targets and accumulates in a MET-expressing xenograft. FIG. 12A. [89Zr]Zr-1E7-Fc can detect MET-positive HNSCC cancer xenograft tumors (white circle) by PET/CT imaging. Mice (n=4) received approximately 9 MBq of [⁸⁹Zr]Zr-1E7-Fc via tail vein and imaged at labeled time points. FIG. 12B. Quantitative analysis of ROI were drawn and evaluated at designated time points for tumor and heart. FIG. 12C. Biodistribution of [⁸⁹Zr]Zr-1E7-Fc at 72 hours was generated from radioactive counts measured from harvested organs and tissues.

### DETAILED DESCRIPTION OF THE INVENTION

The present invention provides a single-domain antibody (sdAb) specifically recognizing mesenchymal epithelial transition factor receptor (MET) (hereinafter also referred to as “anti-MET sdAb”) and its antibody variants, including but not limited to, a larger protein or polypeptide comprising the anti-MET sdAb, such as a heavy chain-only antibody (HCAb), or an anti-MET sdAb fused to a full-length antibody or an antigen-binding fragment thereof, as a new strategy to treat MET-related diseases, such as cancer.

Single-chain antibodies (sdAbs) are different from conventional 4-chain antibodies by having a single monomeric antibody variable domain, such as heavy chain variable domain (VHH, also abbreviated in the art as VHH), which can exhibit high affinity to an antigen without the aid of a light chain. Camelid VHH is known as the smallest functional antigen-binding fragment with a molecular weight of approximately 15 kD.

Accordingly, one aspect of the present application provides an isolated anti-MET construct comprising a sdAb moiety specifically recognizing MET. The isolated anti-MET construct can be, for example, an anti-MET sdAb, a polypeptide comprising multiple anti-MET sdAbs described herein fused together, an HCAb comprising an anti-MET sdAb described herein fused to a human IgG1 Fc, or an anti-MET sdAb fused to a full-length antibody, such as an anti-MET antibody, or an antigen-binding fragment thereof. The anti-MET construct can be monospecific or multispecific, monovalent or multivalent.

Also provided are compositions (such as pharmaceutical compositions), kits and articles of manufacture comprising the construct comprising an anti-MET sdAb moiety, methods of making the construct comprising an anti-MET sdAb moiety, and methods of treating MET-related disease (such as cancer) using the construct comprising an anti-MET sdAb moiety.

### Definitions

The terms “MET,” “c-Met,” “mesenchymal epithelial transition factor receptor,” “mesenchymal epithelial transition,” “mesenchymal epithelial transition receptor,” “mesenchymal epithelial transition factor,” and “hepatocyte growth factor receptor” are used interchangeably, and include variants, isoforms, splice variants, and species homologs of human MET. The canonical sequence for human MET is SEQ ID NO:30. Accordingly, the anti-MET construct of the invention can, in certain cases, cross-react with MET from species other than human, or other proteins which are structurally related to human MET (e.g., human MET homologs). In other cases, the anti-MET construct can be completely specific for human MET and not exhibit species or other types of cross-reactivity.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of cancer. The methods of the invention contemplate any one or more of these aspects of treatment.

The term “therapeutically effective amount” used herein refers to an amount of an agent, a combination of agents, or a pharmaceutical composition comprising such agents sufficient to treat a specified disorder, condition, or disease such as to ameliorate, palliate, lessen, and/or delay one or more of its symptoms. In reference to cancer, a therapeutically effective amount comprises an amount sufficient to cause a tumor to shrink and/or to decrease the growth rate of the tumor (such as to suppress tumor growth) or to prevent or delay other unwanted cell proliferation. In some embodiments, a therapeutically effective amount is an amount sufficient to delay development. In some embodiments, an effective amount is an amount sufficient to prevent or delay recurrence. A therapeutically effective amount can be administered in one or more administrations. The therapeutically effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

“Screening amount” used herein refers to an amount of an agent, a combination of agents, or a pharmaceutical composition comprising such agents sufficient to select a subject for treatment, such as an amount for the agent to bind to a cancer cell or solid tumor in the subject and subsequently be detected at the location of the cancer cell or solid tumor, e.g., by imaging the subject using gamma camera imaging such as planar gamma camera imaging, single photon emission computed tomography or positron emission tomography, optionally combined with a non-nuclear imaging technique such as X-ray imaging, computed tomography and/or magnetic resonance imaging. In some embodiments, a screening amount is an amount that is not therapeutically effective. In some embodiments, the screening amount is different than (e.g., lower than) a “therapeutically effective amount” for treatment as described herein.

As used herein, “imaging an individual” refers to capturing one or more images of an individual using a device that is capable of detecting a labeled (e.g., radiolabeled) construct as described herein. The one or more images may be further altered by a computer program and/or a person skilled in the art in order to enhance the images (e.g., by adjusting contrast or brightness of the one or more images). Any device capable of detecting a labeled (e.g., radiolabeled) construct as described herein is contemplated for use, such as a device for gamma camera imaging such as planar gamma camera imaging, for single photon emission computed tomography or for positron emission tomography, or a device able to combine a nuclear imaging technique with a non-nuclear imaging technique such as X-ray imaging, computed tomography and/or magnetic resonance imaging. For example, such device can be a device for single photon emission computed tomography/computed tomography (SPECT/CT) imaging. Such devices are known in the art and commercially available.

The term “antibody” or “antibody moiety” is used in the broadest sense and encompasses various antibody structures, including but not limited to monoclonal antibodies, polyclonal antibodies, multi specific antibodies (e.g., bispecific antibodies), full-length antibodies and antigen-binding fragments thereof, so long as they exhibit the desired antigen-binding activity.

The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen-binding sites, while IgA antibodies comprise from 2-5 of the basic 4-chain units which can polymerize to form polyvalent assemblages in combination with the J chain. In the case of IgGs, the 4-chain unit is generally about 150,000 Daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (CH) for each of the α and γ chains and four C_{H} domains for μ and ε isotypes. Each L chain has at the N-terminus, a variable domain (V_{L}) followed by a constant domain at its other end. The V_{L} is aligned with the V_{H} and the CL is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see e.g., Basic and Clinical Immunology, 8th Edition, Daniel P. Sties, Abba I. Terr and Tristram G. Parsolw (eds), Appleton & Lange, Norwalk, Conn., 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG and IgM, having heavy chains designated α, δ, ε, γ and μ, respectively. The γ and α classes are further divided into subclasses on the basis of relatively minor differences in the C_{H} sequence and function, e.g., humans express the following subclasses: IgG1, IgG2A, IgG2B, IgG3, IgG4, IgA1 and IgA2.

The term “heavy chain-only antibody” or “HCAb” refers to a functional antibody, which comprises heavy chains, but lacks the light chains usually found in 4-chain antibodies. Camelid animals (such as camels, llamas, or alpacas) are known to produce HCAbs.

The term “single-domain antibody” or “sdAb” refers to a single antigen-binding polypeptide having three complementary determining regions (CDRs). The sdAb alone is capable of binding to the antigen without pairing with a corresponding CDR-containing polypeptide. In some cases, single-domain antibodies are engineered from camelid HCAbs, and their heavy chain variable domains are referred herein as “VHHs” (variable domain of the heavy chain of the heavy chain antibody). Some VHHs can also be known as nanobodies. Camelid sdAb is one of the smallest known antigen-binding antibody fragments (see, e.g., Hamers-Casterman et al., Nature 363:446-8 (1993); Greenberg et al., Nature 374:168-73 (1995); Hassanzadeh-Ghassabeh et al., Nanomedicine (Lond), 8:1013-26 (2013)). A basic VHH has the following structure from the N-terminus to the C-terminus: FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3.

An “isolated” antibody (or construct) is one that has been identified, separated and/or recovered from a component of its production environment (e.g., natural or recombinant). Preferably, the isolated polypeptide is free of association with all other components from its production environment. Contaminant components of its production environment, such as that resulting from recombinant transfected cells, are materials that would typically interfere with research, diagnostic or therapeutic uses for the antibody, and may include enzymes, hormones, and other proteinaceous or non-proteinaceous solutes. In preferred embodiments, the polypeptide will be purified: (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of a spinning cup sequenator; or (3) to homogeneity by SDS-PAGE under non-reducing or reducing conditions using Coomassie Blue or, preferably, silver stain. Isolated antibody (or construct) includes the antibody in situ within recombinant cells since at least one component of the antibody's natural environment will not be present. Ordinarily, however, an isolated polypeptide, antibody, or construct will be prepared by at least one purification step.

The “variable region” or “variable domain” of an antibody refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites. Heavy-chain only antibodies from the Camelid species have a single heavy chain variable region, which is referred to as “VHH”. VHH is thus a special type of V_{H}.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called complementary determining regions (CDRs) or hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g. U.S. Pat. No. 4,816,567), phage-display technologies (see, e.g., Clackson et al., Nature, 352:624-628 (1991); Marks et al., J. Mol. Biol. 222:581-597 (1992); Sidhu et al., J. Mol. Biol. 338 (2): 299-310 (2004); Lee et al., J. Mol. Biol. 340 (5): 1073-1093 (2004); Fellouse, Proc. Natl. Acad. Sci. USA 101 (34): 12467-12472 (2004); and Lee et al., J. Immunol. Methods 284 (1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90:2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10:779-783 (1992); Lonberg et al., Nature 368:856-859 (1994); Morrison, Nature 368:812-813 (1994); Fishwild et al., Nature Biotechnol. 14:845-851 (1996); Neuberger, Nature Biotechnol. 14:826 (1996); and Lonberg and Huszar, Intern. Rev. Immunol. 13:65-93 (1995).

The terms “full-length antibody”, “intact antibody”, or “whole antibody” are used interchangeably to refer to an antibody in its substantially intact form, as opposed to an antibody fragment. Specifically, full-length 4-chain antibodies include those with heavy and light chains including an Fc region. Full-length heavy-chain only antibodies include the heavy chain (such as VHH) and an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, preferably the antigen binding and/or the variable region of the intact antibody. Examples of antibody fragments include, but are not limited to Fab, Fab′, F(ab′)₂ and Fv fragments; diabodies; linear antibodies (see U.S. Pat. No. 5,641,870, Example 2; Zapata et al., Protein Eng. 8 (10): 1057-1062) [1995]; single-chain antibody molecules; single-domain antibodies (such as VHH), and multispecific antibodies formed from antibody fragments. Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)2 fragment which roughly corresponds to two disulfide linked Fab fragments having different antigen-binding activity and is still capable of cross-linking antigen. Fab′ fragments differ from Fab fragments by having a few additional residues at the carboxy-terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

The term “constant domain” refers to the portion of an immunoglobulin molecule having a more conserved amino acid sequence relative to the other portion of the immunoglobulin, the variable domain, which contains the antigen-binding site. The constant domain contains the C_{H}1, C_{H}2 and C_{H}3 domains (collectively, CH) of the heavy chain and the CHL (or CL) domain of the light chain.

The “light chains” of antibodies (immunoglobulins) from any mammalian species can be assigned to one of two clearly distinct types, called kappa (“x”) and lambda (“k”), based on the amino acid sequences of their constant domains.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

“Functional fragments” of the antibodies described herein comprise a portion of an intact antibody, generally including the antigen binding or variable region of the intact antibody or the Fc region of an antibody which retains or has modified FcR binding capability. Examples of antibody fragments include linear antibody, single-chain antibody molecules and multispecific antibodies formed from antibody fragments.

The term “diabodies” refers to small antibody fragments prepared by constructing sFv fragments (see preceding paragraph) with short linkers (about 5-10 residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 93/11161; Hollinger et al., Proc. Natl. Acad. Sci. USA 90:6444-6448 (1993).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is (are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). “Humanized antibody” is used as a subset of “chimeric antibodies”.

“Humanized” forms of non-human (e.g., llama or camelid) antibodies are antibodies that contain minimal sequence derived from non-human immunoglobulin. In some embodiments, a humanized antibody is a human immunoglobulin (recipient antibody) in which residues from an CDR (hereinafter defined) of the recipient are replaced by residues from an CDR of a non-human species (donor antibody) such as mouse, rat, rabbit, camel, llama, alpaca, or non-human primate having the desired specificity, affinity, and/or capacity. In some instances, framework (“FR”) residues of the human immunoglobulin are replaced by corresponding non-human residues. Furthermore, humanized antibodies can comprise residues that are not found in the recipient antibody or in the donor antibody. These modifications can be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR is typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147 (1): 86-95 (1991). See also van Dijk and van de Winkel, Curr. Opin. Pharmacol., 5:368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

The term “hypervariable region,” “HVR,” or “HV,” when used herein refers to the regions of an antibody variable domain which are hypervariable in sequence and/or form structurally defined loops. Generally, single-domain antibodies comprise three HVRs (or CDRs): HVR1 (or CDR1), HVR2 (or CDR2), and HVR3 (or CDR3). HVR3 (or CDR3) displays the most diversity of the three HVRs, and is believed to play a unique role in conferring fine specificity to antibodies. See, e.g., Hamers-Casterman et al., Nature 363:446-448 (1993); Sheriff et al., Nature Struct. Biol. 3:733-736 (1996).

The term “Complementarity Determining Region” or “CDR” is used to refer to hypervariable regions as defined by the Kabat system. See Kabat et al., Sequences of Proteins of Immunological Interest, 5th Ed. Public Health Service, National Institutes of Health, Bethesda, Md. (1991).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk, J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below in Table 1.

TABLE 1
HVR delineations.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |

(Kabat Numbering)

| | | | | |
|---|---|---|---|---|
| H1 | H31-H35 | H26-H35 | H26-H32 | H30-H35 |

(Chothia Numbering)

| | | | | |
|---|---|---|---|---|
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the V_{L} and 26-35 (H1), 50-65 or 49-65 (H2) and 93-102, 94-102, or 95-102 (H3) in the V_{H}. The variable domain residues are numbered according to Kabat et al., supra, for each of these definitions.

The amino acid residues of a single-domain antibody (such as VHH) are numbered according to the general numbering for V_{H} domains given by Kabat et al. (“Sequence of proteins of immunological interest”, US Public Health Services, NIH Bethesda, Md., Publication No. 91), as applied to VHH domains from Camelids in the article of Riechmann and Muyldermans, J. Immunol. Methods 2000 Jun. 23; 240 (1-2): 185-195. According to this numbering, FR1 of a VHH comprises the amino acid residues at positions 1-30, CDR1 of a VHH comprises the amino acid residues at positions 31-35, FR2 of a VHH comprises the amino acids at positions 36-49, CDR2 of a VHH comprises the amino acid residues at positions 50-65, FR3 of a VHH comprises the amino acid residues at positions 66-94, CDR3 of a VHH comprises the amino acid residues at positions 95-102, and FR4 of a VHH comprises the amino acid residues at positions 103-113. In this respect, it should be noted that—as is well known in the art for V_{H} domains and for VHH domains—the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering).

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

Unless indicated otherwise herein, the numbering of the residues in an immunoglobulin heavy chain is that of the EU index as in Kabat et al., supra. The “EU index as in Kabat” refers to the residue numbering of the human IgG1 EU antibody.

“Framework” or “FR” residues are those variable-domain residues other than the HVR residues as herein defined.

A “human consensus framework” or “acceptor human framework” is a framework that represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. Generally, the subgroup of sequences is a subgroup as in Kabat et al., Sequences of Proteins of Immunological Interest, 5th Ed. Public Health Service, National Institutes of Health, Bethesda, Md. (1991). Examples include for the V_{L}, the subgroup may be subgroup kappa I, kappa II, kappa III or kappa IV as in Kabat et al., supra. Additionally, for the VH, the subgroup may be subgroup I, subgroup II, or subgroup III as in Kabat et al. Alternatively, a human consensus framework can be derived from the above in which particular residues, such as when a human framework residue is selected based on its homology to the donor framework by aligning the donor framework sequence with a collection of various human framework sequences. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less.

An “affinity-matured” antibody is one with one or more alterations in one or more CDRs thereof that result in an improvement in the affinity of the antibody for antigen, compared to a parent antibody that does not possess those alteration(s). In some embodiments, an affinity-matured antibody has nanomolar or even picomolar affinities for the target antigen. Affinity-matured antibodies are produced by procedures known in the art. For example, Marks et al., Bio/Technology 10:779-783 (1992) describes affinity maturation by V_{H}- and V_{L}-domain shuffling. Random mutagenesis of CDR and/or framework residues is described by, for example: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154 (7): 3310-9 (1995); and Hawkins et al, J. Mol. Biol. 226:889-896 (1992).

As used herein, the term “specifically binds,” “specifically recognizes,” or is “specific for” refers to measurable and reproducible interactions such as binding between a target and an antigen binding protein (such as a sdAb), which is determinative of the presence of the target in the presence of a heterogeneous population of molecules including biological molecules. For example, an antigen binding protein (such as a sdAb) that specifically binds a target (which can be an epitope) is an antigen binding protein (such as a sdAb) that binds this target with greater affinity, avidity, more readily, and/or with greater duration than it binds other targets. In some embodiments, the extent of binding of an antigen binding protein (such as a sdAb) to an unrelated target is less than about 10% of the binding of the antigen binding protein (such as sdAb) to the target as measured, e.g., by a radioimmunoassay (RIA). In some embodiments, an antigen binding protein (such as a sdAb) that specifically binds a target has a dissociation constant (K_{d}) of ≤10⁻⁵M, ≤10⁻⁶M, ≤10⁻⁷M, ≤10⁻⁸M, ≤10⁻⁹M, ≤10⁻¹⁰M, ≤10⁻¹¹ M, or ≤10⁻¹² M. In some embodiments, an antigen binding protein specifically binds an epitope on a protein that is conserved among the protein from different species. In some embodiments, specific binding can include, but does not require exclusive binding.

The term “specificity” refers to selective recognition of an antigen binding protein (such as a sdAb) for a particular epitope of an antigen. Natural antibodies, for example, are monospecific. The term “multispecific” as used herein denotes that an antigen binding protein has polyepitopic specificity (i.e., is capable of specifically binding to two, three, or more, different epitopes on one biological molecule or is capable of specifically binding to epitopes on two, three, or more, different biological molecules). “Bispecific” as used herein denotes that an antigen binding protein has two different antigen-binding specificities. The term “monospecific” as used herein denotes an antigen binding protein (such as a sdAb) that has one or more binding sites each of which bind the same epitope of the same antigen.

The term “valent” as used herein denotes the presence of a specified number of binding sites in an antigen binding protein. A natural antibody for example or a full length antibody has two binding sites and is bivalent. As such, the terms “trivalent”, “tetravalent”, “pentavalent” and “hexavalent” denote the presence of two binding site, three binding sites, four binding sites, five binding sites, and six binding sites, respectively, in an antigen binding protein.

“Antibody effector functions” refer to those biological activities attributable to the Fc region (a native sequence Fc region or amino acid sequence variant Fc region) of an antibody, and vary with the antibody isotype. Examples of antibody effector functions include: Clq binding and complement dependent cytotoxicity; Fc receptor binding; antibody—dependent cell-mediated cytotoxicity (ADCC); phagocytosis; down regulation of cell surface receptors (e.g., B cell receptors); and B cell activation. “Reduced or minimized” antibody effector function means that which is reduced by at least 50% (alternatively 60%, 65%, 70%, 75%, 80%, 85%, 90%, 95%, 96%, 97%, 98%, 99%) from the wild type or unmodified antibody. The determination of antibody effector function is readily determinable and measurable by one of ordinary skill in the art. In a preferred embodiment, the antibody effector functions of complement binding, complement dependent cytotoxicity and antibody dependent cytotoxicity are affected. In some embodiments, effector function is eliminated through a mutation in the constant region that eliminated glycosylation, e.g., “effector-less mutation.” In one aspect, the effector-less mutation is an N297A or DANA mutation (D265A+N297A) in the C_{H}2 region. Shields et al., J. Biol. Chem. 276 (9): 6591-6604 (2001). Alternatively, additional mutations resulting in reduced or eliminated effector function include: K322A and L234A/L235A (LALA). Alternatively, effector function can be reduced or eliminated through production techniques, such as expression in host cells that do not glycosylate (e.g., E. coli) or in which result in an altered glycosylation pattern that is ineffective or less effective at promoting effector function (e.g., Shinkawa et al., J. Biol. Chem. 278 (5): 3466-3473 (2003).

“Antibody-dependent cell-mediated cytotoxicity” or ADCC refers to a form of cytotoxicity in which secreted Ig bound onto Fc receptors (FcRs) present on certain cytotoxic cells (e.g., natural killer (NK) cells, neutrophils and macrophages) enable these cytotoxic effector cells to bind specifically to an antigen-bearing target cell and subsequently kill the target cell with cytotoxins. The antibodies “arm” the cytotoxic cells and are required for killing of the target cell by this mechanism. The primary cells for mediating ADCC, NK cells, express FcγRIII only, whereas monocytes express FcγRI, FcγRII and FcγRIII Fc expression on hematopoietic cells is summarized in Table 3 on page 464 of Ravetch and Kinet, Annu. Rev. Immunol. 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and natural killer (NK) cells. Alternatively, or additionally, ADCC activity of the molecule of interest may be assessed in vivo, e.g., in an animal model such as that disclosed in Clynes et al., PNAS USA 95:652-656 (1998).

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies described herein include human IgG1, IgG2 (IgG2A, IgG2B), IgG3 and IgG4. “Fc receptor” or “FcR” describes a receptor that binds the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see M. Daeron, Annu. Rev. Immunol. 15: 203-234 (1997). FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus. Guyer et al., J. Immunol. 117:587 (1976) and Kim et al., J. Immunol. 24:249 (1994). Methods of measuring binding to FcRn are known (see, e.g., Ghetie and Ward, Immunol. Today 18: (12): 592-8 (1997); Ghetie et al., Nature Biotechnology 15 (7): 637-40 (1997); Hinton et al., J. Biol. Chem. 279 (8): 6213-6 (2004); WO 2004/92219 (Hinton et al.). Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody variants which improved or diminished binding to FcRs. See also, e.g., Shields et al., J. Biol. Chem. 9 (2): 6591-6604 (2001).

“Complement dependent cytotoxicity” or “CDC” refers to the lysis of a target cell in the presence of complement. Activation of the classical complement pathway is initiated by the binding of the first component of the complement system (Clq) to antibodies (of the appropriate subclass) which are bound to their cognate antigen. To assess complement activation, a CDC assay, e.g., as described in Gazzano-Santoro et al., J. Immunol. Methods 202:163 (1996), may be performed. Antibody variants with altered Fc region amino acid sequences and increased or decreased Clq binding capability are described in U.S. Pat. No. 6,194,551B1 and WO99/51642. The contents of those patent publications are specifically incorporated herein by reference. See, also, Idusogie et al. J. Immunol. 164:4178-4184 (2000). “Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity that reflects a 1:1 interaction between members of a binding pair. Binding affinity can be indicated by K_{d}, K_{off}, Kₒₙ, or Kₐ. The term “K_{off}”, as used herein, is intended to refer to the off rate constant for dissociation of an antibody (or antigen-binding domain) from the antibody/antigen complex, as determined from a kinetic selection set up, expressed in units of s⁻¹. The term “Kₒₙ”, as used herein, is intended to refer to the on rate constant for association of an antibody (or antigen-binding domain) to the antigen to form the antibody/antigen complex, expressed in units of M⁻¹s⁻¹. The term equilibrium dissociation constant “K_{D}” or “K_{d}”, as used herein, refers to the dissociation constant of a particular antibody-antigen interaction, and describes the concentration of antigen required to occupy one half of all of the antibody-binding domains present in a solution of antibody molecules at equilibrium, and is equal to K_{off}/Kₒₙ, expressed in units of M. The measurement of Kₐ presupposes that all binding agents are in solution. In the case where the antibody is tethered to a cell wall, e.g., in a yeast expression system, the corresponding equilibrium rate constant is expressed as EC50, which gives a good approximation of K_{d}. The affinity constant, Kₐ, is the inverse of the dissociation constant, K_{d}, expressed in units of M⁻¹.

The dissociation constant (K_{D} or K_{d}) is used as an indicator showing affinity of antibodies to antigens. For example, easy analysis is possible by the Scatchard method using antibodies marked with a variety of marker agents, as well as by using BiacoreX (made by Amersham Biosciences), which is an over-the-counter, measuring kit, or similar kit, according to the user's manual and experiment operation method attached with the kit. The K_{D} value that can be derived using these methods is expressed in units of M (Mols). An antibody or antigen-binding fragment thereof that specifically binds to a target may have a dissociation constant (K_{d}) of, for example, ≤10⁻⁵M, ≤10⁻⁶M, ≤10⁻⁷M, ≤10⁻⁸M, ≤10⁻⁹M, ≤10⁻¹⁰M, ≤10⁻¹⁰ M, ≤10⁻¹¹ M, or ≤10⁻¹² M.

Binding specificity of the antibody or antigen-binding domain can be determined experimentally by methods known in the art. Such methods comprise, but are not limited to Western blots, ELISA-, RIA-, ECL-, IRMA-, EIA-, BIAcore-tests and peptide scans.

Half maximal inhibitory concentration (IC₅₀) is a measure of the effectiveness of a substance (such as an antibody) in inhibiting a specific biological or biochemical function. It indicates how much of a particular drug or other substance (inhibitor, such as an antibody) is needed to inhibit a given biological process (e.g., the binding between MET and B7-1, or component of a process, i.e. an enzyme, cell, cell receptor or microorganism) by half. The values are typically expressed as molar concentration. IC₅₀ is comparable to an EC50 for agonist drug or other substance (such as an antibody). EC₅₀ also represents the plasma concentration required for obtaining 50% of a maximum effect in vivo. As used herein, an “IC₅₀” is used to indicate the effective concentration of an antibody (such as an anti-MET sdAb) needed to neutralize 50% of the antigen bioactivity (such as MET bioactivity) in vitro. IC₅₀ or EC₅₀ can be measured by bioassays such as inhibition of ligand binding by FACS analysis (competition binding assay), cell based cytokine release assay, or amplified luminescent proximity homogeneous assay (AlphaLISA).

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An “isolated” nucleic acid molecule encoding a construct, antibody, or antigen-binding fragment thereof described herein is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies described herein is in a form other than in the form or setting in which it is found in nature. Isolated nucleic acid molecules therefore are distinguished from nucleic acid encoding the polypeptides and antibodies described herein existing naturally in cells. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “radiolabeled,” as in a “radiolabeled” amino acid sequence, radiolabeled” antibody fragment, or “radiolabeled” VHH, refers to the radioisotopic labeling of that amino acid sequence, antibody fragment or VHH, wherein the amino acid sequence, antibody fragment or VHH is labelled by including, coupling, or chemically linking a radionuclide to its amino acid sequence structure.

The terms “radionuclide,” “radioactive nuclide,” “radioisotope,” and “radioactive isotope” are used interchangeably herein and refer to atoms with an unstable nucleus, characterized by excess energy available to be imparted either to a newly created radiation particle within the nucleus or via internal conversion. During this process, the radionuclide is said to undergo radioactive decay, resulting in the emission of gamma ray(s) and/or subatomic particles such as alpha or beta particles. These emissions constitute ionizing radiation. Radionuclides occur naturally, or can be produced artificially. In some embodiments, the radioisotope is both a “y-emitter and β-emitter”, meaning the radioisotope emits both gamma (Y) rays and beta (B) particles.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

“Adjuvant setting” refers to a clinical setting in which an individual has had a history of cancer, and generally (but not necessarily) been responsive to therapy, which includes, but is not limited to, surgery (e.g., surgery resection), radiotherapy, and chemotherapy. However, because of their history of cancer, these individuals are considered at risk of development of the disease. Treatment or administration in the “adjuvant setting” refers to a subsequent mode of treatment. The degree of risk (e.g., when an individual in the adjuvant setting is considered as “high risk” or “low risk”) depends upon several factors, most usually the extent of disease when first treated.

“Neoadjuvant setting” refers to a clinical setting in which the method is carried out before the primary/definitive therapy.

The term “pharmaceutical formulation” of “pharmaceutical composition” refers to a preparation that is in such form as to permit the biological activity of the active ingredient to be effective, and that contains no additional components that are unacceptably toxic to a subject to which the formulation would be administered. Such formulations are sterile. A “sterile” formulation is aseptic or free from all living microorganisms and their spores.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

### Anti-MET Constructs

### Anti-MET Single-Domain Antibody Moiety

The isolated anti-MET constructs described herein can comprise a single-domain antibody (sdAb) moiety that specifically recognizes MET (or “anti-MET sdAb”). In some embodiments, the isolated anti-MET construct is an anti-MET sdAb.

### Single-Domain Antibodies

Exemplary sdAbs include, but are not limited to, heavy chain variable domains from heavy-chain only antibodies (e.g., VHH (variable domain of the heavy chain of the heavy chain antibody) in Camelidae or V_{NAR} (Variable domain of the shark New Antigen Receptor) in cartilaginous fish), binding molecules naturally devoid of light chains, single domains (such as V_{H} or V_{L}) derived from conventional 4-chain antibodies, humanized heavy-chain only antibodies, human single-domain antibodies produced by transgenic mice or rats expressing human heavy chain segments, and engineered domains and single domain scaffolds other than those derived from antibodies. The sdAbs may be derived from any species including, but not limited to mouse, rat, human, camel, llama, lamprey, fish, shark, goat, rabbit, and bovine. Single-domain antibodies contemplated herein also include naturally occurring single-domain antibody molecules from species other than Camelidae and sharks.

In some embodiments, the sdAb is derived from a naturally occurring single-domain antigen binding molecule known as heavy chain antibody devoid of light chains (also referred herein as “heavy chain-only antibodies”, or “HCAb”). Such single domain molecules are disclosed in WO 94/04678 and Hamers-Casterman, C. et al. (1993) Nature 363:446-448, for example. For clarity reasons, the variable domain derived from a heavy chain molecule naturally devoid of light chain is known herein as a VHH to distinguish it from the conventional VH of four chain immunoglobulins. Such a VHH molecule can be derived from antibodies raised in Camelidae species, for example, camel, llama, vicuna, dromedary, alpaca and guanaco. Other species besides Camelidae may produce heavy chain molecules naturally devoid of light chain, and such VHHs are within the scope of the present application.

In some embodiments, the sdAb is derived from a variable region of the immunoglobulin found in cartilaginous fish. For example, the sdAb can be derived from the immunoglobulin isotype known as Novel Antigen Receptor (NAR) found in the serum of shark. Methods of producing single domain molecules derived from a variable region of NAR (“IgNARs”) are described in WO 03/014161 and Streltsov (2005) Protein Sci. 14:2901-2909. In some embodiments, the sdAb is recombinant, CDR-grafted, humanized, camelized, de-immunized and/or in vitro generated (e.g., selected by phage display). In some embodiments, the amino acid sequence of the framework regions may be altered by “camelization” of specific amino acid residues in the framework regions. Camelization refers to the replacement or substitution of one or more amino acid residues in the amino acid sequence of a (naturally occurring) VH domain from a conventional 4-chain antibody by one or more of the amino acid residues that occur at the corresponding position(s) in a VHH domain of a heavy chain antibody. This can be performed in a manner known per se, which will be clear to the skilled person, for example on the basis of the further description herein. Such “camelizing” substitutions are preferably inserted at amino acid positions that form and/or are present at the VH-VL interface, and/or at the so-called Camelidae hallmark residues, as defined herein (see for example WO 94/04678, Davies and Riechmann FEBS Letters 339:285-290, 1994; Davies and Riechmann Protein Engineering 9 (6): 531-537, 1996; Riechmann J. Mol. Biol. 259:957-969, 1996; and Riechmann and Muyldermans J. Immunol. Meth. 231:25-38, 1999).

In some embodiments, the sdAb is a human sdAb produced by transgenic mice or rats expressing human heavy chain segments. See, e.g., US20090307787A1, U.S. Pat. No. 8,754,287, US20150289489A1, US20100122358A1, and WO2004049794. In some embodiments, the sdAb is affinity matured.

In some embodiments, naturally occurring VHH domains against a particular antigen or target, can be obtained from (naïve or immune) libraries of Camelid VHH sequences. Such methods may or may not involve screening such a library using said antigen or target, or at least one part, fragment, antigenic determinant or epitope thereof using one or more screening techniques known per se. Such libraries and techniques are for example described in WO 99/37681, WO 01/90190, WO 03/025020 and WO 03/035694. Alternatively, improved synthetic or semi-synthetic libraries derived from (naïve or immune) VHH libraries may be used, such as VHH libraries obtained from (naïve or immune) VHH libraries by techniques such as random mutagenesis and/or CDR shuffling, as for example described in WO 00/43507.

In some embodiments, the sdAbs are generated from conventional four-chain antibodies. See, for example, EP 0 368 684, Ward et al. (Nature 1989 Oct. 12; 341 (6242): 544-6), Holt et al., Trends Biotechnol., 2003, 21 (11): 484-490; WO 06/030220; and WO 06/003388.

Because of the unique properties of sdAbs, using VHH domains as single antigen-binding proteins or as antigen-binding domains (i.e. as part of a larger protein or polypeptide) offers a number of significant advantages over the conventional V_{H} and V_{L}, scFv and conventional antibody fragments (such as Fab or (Fab′)₂): 1) only a single domain is required to bind an antigen with high affinity, so there is no need to have a second domain, nor to assure that these two domains are present in the correct spatial conformation and configuration (e.g. no need to pair the heavy chain and light chain during folding, no need to use a specially designed linker such as for scFv); 2) VHH domains and other sdAbs can be expressed from a single gene and require no post-translational folding or modifications; 3) VHH domains and other sdAbs can be easily engineered into multivalent and/or multispecific formats (such as those described in the present application); 4) VHH domains and other sdAbs are highly soluble and do not have a tendency to aggregate (as with the mouse-derived “dAbs” described by Ward et al., Nature. 1989 Oct. 12; 341 (6242): 544-6); 5) VHH domains and other sdAbs are highly stable against heat, pH, proteases and other denaturing agents or conditions; 6) VHH domains and other sdAbs are easy and relatively cheap to prepare (even on a large production scale), such as using microbial fermentation, there is no need to use mammalian expression system (required by production of, for example, conventional antibody fragments); 7) VHH domains and other sdAbs are relatively small (approximately 15 kDa, or 10 times smaller than a conventional IgG) compared to conventional 4-chain antibodies and antigen-binding fragments thereof, thus have high(er) tissue penetration ability, such as for solid tumors and other dense tissues; and 8) VHH domains and other sdAbs can exhibit so-called “cavity-binding properties” (due to their extended CDR3 loop compared to that of conventional V_{H} domains) and can therefore access targets and epitopes not accessible to conventional 4-chain antibodies and antigen-binding fragments thereof, for example, it has been shown that VHH domains and other sdAbs can inhibit enzymes (see for example WO1997049805; Transue et al., Proteins. 1998 Sep. 1; 32 (4): 515-22; Lauwereys et al., EMBO J. 1998 Jul. 1; 17 (13): 3512-20).

### MET

MET is a receptor tyrosine kinase (RTK) that is produced as a single-chain precursor. The precursor is proteolytically cleaved at a furin site to yield a highly glycosylated extracellular a-subunit and a transmembrane β-subunit, which are linked together by a disulfide bridge. The extracellular domain includes the full a-chain and the N-terminal part of the β-chain.

The amino acid sequence of the canonical human MET protein is:

(SEQ ID NO: 30)
MKAPAVLAPGILVLLFTLVQRSNGECKEALAKSEMNVNMKYQLPN
FTAETPIQNVILHEHHIFLGATNYIYVLNEEDLQKVAEYKTGPVL
EHPDCFPCQDCSSKANLSGGVWKDNINMALVVDTYYDDQLISCGS
VNRGTCQRHVFPHNHTADIQSEVHCIFSPQIEEPSQCPDCVVSAL
GAKVLSSVKDRFINFFVGNTINSSYFPDHPLHSISVRRLKETKDG
FMFLTDQSYIDVLPEFRDSYPIKYVHAFESNNFIYFLTVQRETLD
AQTFHTRIIRFCSINSGLHSYMEMPLECILTEKRKKRSTKKEVFN
ILQAAYVSKPGAQLARQIGASLNDDILFGVFAQSKPDSAEPMDRS
AMCAFPIKYVNDFENKIVNKNNVRCLQHFYGPNHEHCFNRTLLRN
SSGCEARRDEYRTEFTTALQRVDLFMGQFSEVLLTSISTFIKGDL
TIANLGTSEGRFMQVVVSRSGPSTPHVNFLLDSHPVSPEVIVEHT
LNQNGYTLVITGKKITKIPLNGLGCRHFQSCSQCLSAPPFVQCGW
CHDKCVRSEECLSGTWTQQICLPAIYKVFPNSAPLEGGTRLTICG
WDFGFRRNNKFDLKKTRVLLGNESCTLTLSESTMNTLKCTVGPAM
NKHENMSIIISNGHGTTQYSTFSYVDPVITSISPKYGPMAGGTLL
TLTGNYLNSGNSRHISIGGKTCTLKSVSNSILECYTPAQTISTEF
AVKLKIDLANRETSIFSYREDPIVYEIHPTKSFISGGSTITGVGK
NLNSVSVPRMVINVHEAGRNFTVACQHRSNSEIICCTTPSLQQLN
LQLPLKTKAFFMLDGILSKYFDLIYVHNPVFKPFEKPVMISMGNE
NVLEIKGNDIDPEAVKGEVLKVGNKSCENIHLHSEAVLCTVPNDL
LKLNSELNIEWKQAISSTVLGKVIVQPDQNFTGLIAGVVSISTAL
LLLLGFFLWLKKRKQIKDLGSELVRYDARVHTPHLDRLVSARSVS
PTTEMVSNESVDYRATFPEDQFPNSSQNGSCRQVQYPLTDMSPIL
TSGDSDISSPLLQNTVHIDLSALNPELVQAVQHVVIGPSSLIVHF
NEVIGRGHFGCVYHGTLLDNDGKKIHCAVKSLNRITDIGEVSQFL
TEGIIMKDFSHPNVLSLLGICLRSEGSPLVVLPYMKHGDLRNFIR
NETHNPTVKDLIGFGLQVAKGMKYLASKKFVHRDLAARNCMLDEK
FTVKVADEGLARDMYDKEYYSVHNKTGAKLPVKWMALESLQTQKF
TTKSDVWSFGVLLWELMTRGAPPYPDVNTFDITVYLLQGRRLLQP
EYCPDPLYEVMLKCWHPKAEMRPSFSELVSRISAIFSTFIGEHYV
HVNATYVNVKCVAPYPSLLSSEDNADDEVDTRPASFWETS

Various human MET isoforms are known in the art. A particular human MET sequence will generally be at least 90% identical SEQ ID NO:30 and contain amino acid residues that identify the amino acid sequence as being human when compared to MET amino acid sequences of other species (e.g., murine). In some embodiments, a human MET may be at least about 95%, 96%, 97%, 98%, or 99% identical in amino acid sequence to SEQ ID NO:30. In some embodiments, a human MET sequence will display no more than 10 amino acid differences from SEQ ID NO:30. In some embodiments, the human MET may display no more than 5, 4, 3, 2, or 1 amino acid difference SEQ ID NO:30. Percent identity can be determined as described herein. In some embodiments, the anti-MET sdAb moiety described herein specifically recognizes a MET polypeptide with 100% amino acid sequence identity to SEQ ID NO: 30.

In some embodiments, the anti-MET sdAb moiety may cross-react with MET from species other than human, or other proteins which are structurally related to human MET (e.g., human MET homologs). In some embodiments, the anti-MET sdAb moiety is completely specific for human MET and does not exhibit species or other types of cross-reactivity. In some embodiments, the anti-MET sdAb moiety specifically recognizes a soluble isoform of human MET.

In some embodiments, the anti-MET sdAb moiety described herein specifically recognizes the extracellular domain (ECD) of MET. In some embodiments, the anti-MET sdAb moiety specifically recognizes the N-terminal portion of the MET extracellular domain (ECD). In some embodiments, the anti-MET sdAb moiety specifically recognizes the C-terminal portion of the MET extracellular domain (ECD). In some embodiments, the anti-MET sdAb moiety specifically recognizes the middle portion of the MET extracellular domain (ECD).

In some embodiments, the anti-MET sdAb moiety described herein specifically recognizes human METex14. METex14 is a MET protein in which exon 14 is lacking due to exon 14 skipping. The amino acid sequence of an exemplary human METex 14 protein is (MET Exon 14 NP_000236.2):

(SEQ ID NO: 31)
MKAPAVLAPGILVLLFTLVQRSNGECKEALAKSEMNVNMKYQLPN
FTAETPIQNVILHEHHIFLGATNYIYVLNEEDLQKVAEYKTGPVL
EHPDCFPCQDCSSKANLSGGVWKDNINMALVVDTYYDDQLISCGS
VNRGTCQRHVFPHNHTADIQSEVHCIFSPQIEEPSQCPDCVVSAL
GAKVLSSVKDRFINFFVGNTINSSYFPDHPLHSISVRRLKETKDG
FMFLTDQSYIDVLPEFRDSYPIKYVHAFESNNFIYFLTVQRETLD
AQTFHTRIIRFCSINSGLHSYMEMPLECILTEKRKKRSTKKEVFN
ILQAAYVSKPGAQLARQIGASLNDDILFGVFAQSKPDSAEPMDRS
AMCAFPIKYVNDFFNKIVNKNNVRCLQHFYGPNHEHCENRTLLRN
SSGCEARRDEYRTEFTTALQRVDLFMGQFSEVLLTSISTFIKGDL
TIANLGTSEGREMQVVVSRSGPSTPHVNFLLDSHPVSPEVIVEHT
LNQNGYTLVITGKKITKIPLNGLGCRHFQSCSQCLSAPPFVQCGW
CHDKCVRSEECLSGTWTQQICLPAIYKVEPNSAPLEGGTRLTICG
WDFGFRRNNKFDLKKTRVLLGNESCTLTLSESTMNTLKCTVGPAM
NKHFNMSIIISNGHGTTQYSTFSYVDPVITSISPKYGPMAGGTLL
TLTGNYLNSGNSRHISIGGKTCTLKSVSNSILECYTPAQTISTEF
AVKLKIDLANRETSIFSYREDPIVYEIHPTKSFISGGSTITGVGK
NLNSVSVPRMVINVHEAGRNFTVACQHRSNSEIICCTTPSLQQLN
LQLPLKTKAFFMLDGILSKYFDLIYVHNPVFKPFEKPVMISMGNE
NVLEIKGNDIDPEAVKGEVLKVGNKSCENIHLHSEAVLCTVPNDL
LKLNSELNIEWKQAISSTVLGKVIVQPDQNFTGLIAGVVSISTAL
LLLLGFFLWLKKRKQIKDLGSELVRYDARVHTPHLDRLVSARSVS
PTTEMVSNESVDYRATFPEDQFPNSSQNGSCRQVQYPLTDMSPIL
TSGDSDISSPLLQNTVHIDLSALNPELVQAVQHVVIGPSSLIVHF
NEVIGRGHFGCVYHGTLLDNDGKKIHCAVKSLNRITDIGEVSQFL
TEGIIMKDESHPNVLSLLGICLRSEGSPLVVLPYMKHGDLRNFIR
NETHNPTVKDLIGFGLQVAKGMKYLASKKFVHRDLAARNCMLDEK
FTVKVADFGLARDMYDKEYYSVHNKTGAKLPVKWMALESLQTQKF
TTKSDVWSFGVLLWELMTRGAPPYPDVNTFDITVYLLQGRRLLQP
EYCPDPLYEVMLKCWHPKAEMRPSFSELVSRISAIFSTFIGEHYV
HVNATYVNVKCVAPYPSLLSSEDNADDEVDTRPASFWETS

### Antibody Affinity

Binding specificity of the antibody or antigen-binding domain can be determined experimentally by methods known in the art. Such methods comprise, but are not limited to Western blots, ELISA-, RIA-, ECL-, IRMA-, EIA-, BIAcore-tests and peptide scans.

In some embodiments, the K_{d} of the binding between the anti-MET sdAb moiety and MET is about 10⁻⁵ M to about 10⁻⁶ M, about 10⁻⁶ M to about 10⁻⁷M, about 10⁻⁷M to about 10⁻⁸ M, about 10⁻⁸ M to about 10⁻⁹M, about 10⁻⁹ M to about 10⁻¹⁰ M, about 10⁻¹⁰ M to about 10⁻¹¹ M, about 10⁻¹¹ M to about 10⁻¹² M, about 10⁻⁵ M to about 10⁻¹² M, about 10⁻⁶ M to about 10⁻¹² M, about 10⁻⁷M to about 10⁻¹² M, about 10⁻⁸ M to about 10⁻¹² M, about 10⁻⁹ M to about 10⁻¹² M, about 10⁻¹⁰ M to about 10⁻¹² M, about 10⁻⁵ M to about 10⁻¹¹ M, about 10⁻⁷M to about 10⁻¹¹ M, about 10⁻⁸ M to about 10⁻¹¹ M, about 10⁻⁹ M to about 10⁻¹¹ M, about 10⁻⁵ M to about 10⁻¹⁰ M, about 10⁻⁷M to about 10⁻¹⁰ M, about 10⁻⁸ M to about 10⁻¹⁰ M, about 10⁻⁵ M to about 10⁻⁹ M, about 10⁻⁷M to about 10⁻⁹ M, about 10⁻⁵ M to about 10⁻⁸ M, or about 10⁻⁶ M to about 10⁻⁸ M.

In some embodiments, the Kₒₙ of the binding between the anti-MET sdAb moiety and MET is about 10² M⁻¹s⁻¹ to about 10⁴ M⁻¹s⁻¹, about 10⁴ M⁻¹s⁻¹ to about 10⁶ M⁻¹s⁻¹, about 10⁶ M⁻¹s⁻¹ to about 10⁷ M⁻¹s⁻¹, about 10² M⁻¹s⁻¹ to about 10⁷M⁻¹s⁻¹, about 10³ M⁻¹s⁻¹ to about 10⁷ M⁻¹s⁻¹, about 10⁴ M⁻¹s⁻¹ to about 10⁷M⁻¹s⁻¹, about 10⁵ M⁻¹s⁻¹ to about 10⁷ M⁻¹s⁻¹, about 10³ M⁻¹s⁻¹ to about 10⁶ M⁻¹s⁻¹, or about 10⁴ M⁻¹s⁻¹ to about 10⁶ M⁻¹s⁻¹.

In some embodiments, the K_{off} of the binding between the anti-MET sdAb moiety and MET is about 1 s⁻¹ to about 10⁻² s⁻¹, about 10⁻² s⁻¹ to about 10⁻⁴ s⁻¹, about 10⁻⁴ s⁻¹ to about 10⁻⁵ s⁻¹, about 10⁻⁵ s⁻¹ to about 10⁶ s⁻¹, about 1 s⁻¹ to about 10⁻⁶ s⁻¹, about 10⁻² s⁻¹ to about 10⁻⁶ s⁻¹, about 10⁻³ s⁻¹ to about 10⁶ s⁻¹, about 10⁻⁴ s⁻¹ to about 10⁶ s⁻¹, about 10⁻² s⁻¹ to about 10⁻⁵ s⁻¹, or about 10⁻³ s⁻¹ to about 10⁻⁵ s⁻¹.

In some embodiments, the IC₅₀ of the anti-MET sdAb moiety is less than 10 nM in an amplified luminescent proximity homogeneous assay (AlphaLISA) with 0.12 nM PD-1 and 0.2 nM MET. In some embodiments, the IC₅₀ of the anti-MET sdAb moiety is less than 500 nM in an inhibition of ligand binding by FACS analysis (competition binding assay), or cell based cytokine release assay. In some embodiments, the IC₅₀ of the anti-MET sdAb moiety is less than 1 nM, about 1 nM to about 10 nM, about 10 nM to about 50 nM, about 50 nM to about 100 nM, about 100 nM to about 200 nM, about 200 nM to about 300 nM, about 300 nM to about 400 nM, or about 400 nM to about 500 nM.

### Chimeric or Humanized Antibodies

In some embodiments, the anti-MET antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a camelid species, such as llama) and a human constant region. In a further example, a chimeric antibody is a “class switched” antibody in which the class or subclass has been changed from that of the parent antibody. Chimeric antibodies include antigen-binding fragments thereof.

In some embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences or are modified to have residues from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); Osbourn et al., Methods 36:61-68 (2005) and Klimka et al., Br. J. Cancer, 83:252-260 (2000) (describing the “guided selection” approach to FR shuffling); Vincke et al. J Biol Chem. 284 (5): 3273-3284 (2009); and Sulea, T. Humanization of Camelid Single-Domain Antibodies. Methods Mol. Biol. 2022, 2446, 299-312.

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

In some embodiments, the sdAbs are modified, such as humanized, without diminishing the native affinity of the domain for antigen and while reducing its immunogenicity with respect to a heterologous species. For example, the amino acid residues of the antibody variable domain (VHH) of a llama antibody can be determined, and one or more of the Camelid amino acids, for example, in the framework regions, are replaced by their human counterpart as found in the human consensus sequence, without that polypeptide losing its typical character, i.e. the humanization does not significantly affect the antigen binding capacity of the resulting polypeptide. Humanization of Camelid single-domain antibodies can be obtained by the introduction and mutagenesis of a limited amount of amino acids in a single polypeptide chain. This is in contrast to humanization of scFv, Fab′, (Fab′)₂ and IgG, which requires the introduction of amino acid changes in two chains, the light and the heavy chain and the preservation of the assembly of both chains.

Single-domain antibodies comprising a VHH domain can be humanized to have human-like sequences. In some embodiments, the FR regions of the VHH domain used herein comprise at least about any one of 50%, 60%, 70%, 80%, 90%, 95% or more of amino acid sequence homology to human VH framework regions. One exemplary class of humanized VHH domains is characterized in that the VHHs carry an amino acid from the group consisting of glycine, alanine, valine, leucine, isoleucine, proline, phenylalanine, tyrosine, tryptophan, methionine, serine, threonine, asparagine, or glutamine at position 45, such as, for example, L45 and a tryptophan at position 103, according to the Kabat numbering. As such, polypeptides belonging to this class show a high amino acid sequence homology to human VH framework regions and said polypeptides might be administered to a human directly without expectation of an unwanted immune response therefrom, and without the burden of further humanization.

Another exemplary class of humanized Camelid single-domain antibodies has been described in WO 03/035694 and contains hydrophobic FR2 residues typically found in conventional antibodies of human origin or from other species, but compensating this loss in hydrophilicity by the charged arginine residue on position 103 that substitutes the conserved tryptophan residue present in V_{H} from double-chain antibodies. As such, peptides belonging to these two classes show a high amino acid sequence homology to human V_{H} framework regions, and said peptides might be administered to a human directly without expectation of an unwanted immune response therefrom and without the burden of further humanization.

### Human Antibodies

In some embodiments, the anti-MET sdAb moiety provided herein is a human antibody. Human antibodies can be produced using various techniques known in the art. Human antibodies are described generally in van Dijk and van de Winkel, Curr. Opin. Pharmacol. 5:368-74 (2001) and Lonberg, Curr. Opin. Immunol. 20:450-459 (2008). Transgenic mice or rats capable of producing fully human single-domain antibodies are known in the art. See, e.g., US20090307787A1, U.S. Pat. No. 8,754,287, US20150289489A1, US20100122358A1, and WO2004049794.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133:3001 (1984); Brodeur et al., Monoclonal Antibody Production Techniques and Applications, pp. 51-63 (Marcel Dekker, Inc., New York, 1987); and Boerner et al., J. Immunol., 147:86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26 (4): 265-268 (2006) (describing human-human hybridomas). Human hybridoma technology (Trioma technology) is also described in Vollmers and Brandlein, Histology and Histopathology, 20 (3): 927-937 (2005) and Vollmers and Brandlein, Methods and Findings in Experimental and Clinical Pharmacology, 27 (3): 185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

One technique for obtaining VHH sequences directed against a particular antigen or target involves suitably immunizing a transgenic mammal that is capable of expressing heavy chain antibodies (i.e. so as to raise an immune response and/or heavy chain antibodies directed against said antigen or target), obtaining a suitable biological sample from said transgenic mammal that contains (nucleic acid sequences encoding) said VHH sequences (such as a blood sample, serum sample or sample of B-cells), and then generating VHH sequences directed against said antigen or target, starting from said sample, using any suitable technique known per se (such as any of the methods described herein or a hybridoma technique). For example, for this purpose, the heavy chain antibody-expressing mice and the further methods and techniques described in WO 02/085945, WO 04/049794 and WO 06/008548 and Janssens et al., Proc. Natl. Acad. Sci. USA. 2006 Oct. 10; 103 (41): 15130-5 can be used. For example, such heavy chain antibody expressing mice can express heavy chain antibodies with any suitable (single) variable domain, such as (single) variable domains from natural sources (e.g. human (single) variable domains, Camelid (single) variable domains or shark (single) variable domains), as well as for example synthetic or semi-synthetic (single) variable domains.

### Library-Derived Antibodies

Antibodies of the present application may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352:624-628 (1991); Marks et al., J. Mol. Biol. 222:581-597 (1992); Marks and Bradbury, in Methods in Molecular Biology 248:161-175 (Lo, ed., Human Press, Totowa, N.J., 2003); Sidhu et al., J. Mol. Biol. 338 (2): 299-310 (2004); Lee et al., J. Mol. Biol. 340 (5): 1073-1093 (2004); Fellouse, Proc. Natl. Acad. Sci. USA 101 (34): 12467-12472 (2004); and Lee et al., J Immunol. Methods 284 (1-2): 119-132 (2004). Methods for constructing single-domain antibody libraries have been described, for example, see U.S. Pat. No. 7,371,849.

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12:433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12:725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J Mol. Biol., 227:381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

### Exemplary Anti-MET Constructs and Elements Thereof

In some embodiments, the sdAb moiety comprises a CDR1, a CDR2, and/or a CDR3. In some embodiments, the sdAb moiety comprises a CDR1, a CDR2, and a CDR3.

In some embodiments, the CDR1 comprises the amino acid sequence of GFX¹FX²X³YDMX⁴ (SEQ ID NO:1), wherein:
X¹ is N or T;
X² is E or G;
X³ is R or A; and
X⁴ is S or V,
   or a variant thereof comprising up to about 3 (such as about any of 1, 2, or 3) amino acid substitutions. In some embodiments, the CDR1 comprises the amino acid sequence of any one of GENFERYDMS (SEQ ID NO:7) and GFTFGAYDMV (SEQ ID NO:8), or a variant thereof comprising up to about 3 (such as about any of 1, 2, or 3) amino acid substitutions. In embodiments, the CDR1 comprises the amino acid sequence of any one of GFNFERYDMS (SEQ ID NO:7) and GFTFGAYDMV (SEQ ID NO:8).

In some embodiments, the CDR2 comprises the amino acid sequence of X⁵X⁶X⁷x⁸X⁹GX¹⁰X¹¹X¹²X¹³, wherein:
X⁵ is R or F;
X⁶ is L or I;
X⁷ is N or S;
X⁸ is S or N;
X⁹ is F or G;
X¹⁰ is R or E;
X¹¹ is S or E;
X¹² is T or V; and
X¹³ is Y or S,
   or a variant thereof comprising up to about 3 (such as about any of 1, 2, or 3) amino acid substitutions. In some embodiments, the CDR2 comprises the amino acid sequence of any one of RLNSFGRSTY (SEQ ID NO:9) and FISNGGEEVS (SEQ ID NO:10), or a variant thereof comprising up to about 3 (such as about any of 1, 2, or 3) amino acid substitutions. In some embodiments, the CDR2 comprises the amino acid sequence of any one of RLNSFGRSTY (SEQ ID NO:9) and FISNGGEEVS (SEQ ID NO:10).

In some embodiments, the CDR3 comprises the amino acid sequence of RSTDVSPGLSSWWTYEYDV (SEQ ID NO:11), or a variant thereof comprising up to about 3 (such as about any of 1, 2, or 3) amino acid substitutions. The CDR3 comprises the amino acid sequence of RSTDVSPGLSSWWTYEYDV (SEQ ID NO:11).

In some embodiments, the sdAb moiety comprises a CDR1 comprising the amino acid sequence of GENFERYDMS (SEQ ID NO:7) or a variant thereof comprising up to about 3 (such as about any of 1, 2, or 3) amino acid substitutions, a CDR2 comprising the amino acid sequence of RLNSFGRSTY (SEQ ID NO:9) or a variant thereof comprising up to about 3 (such as about any of 1, 2, or 3) amino acid substitutions, and a CDR3 comprising the amino acid sequence of RSTDVSPGLSSWWTYEYDV (SEQ ID NO:11) or a variant thereof comprising up to about 3 (such as about any of 1, 2, or 3) amino acid substitutions. In some embodiments, the sdAb moiety comprises a CDR1 comprising the amino acid sequence of GENFERYDMS (SEQ ID NO:7), a CDR2 comprising the amino acid sequence of RLNSFGRSTY (SEQ ID NO:9), and a CDR3 comprising the amino acid sequence of RSTDVSPGLSSWWTYEYDV (SEQ ID NO:11).

In some embodiments, the sdAb moiety comprises a CDR1 comprising the amino acid sequence of GFTFGAYDMV (SEQ ID NO:8) or a variant thereof comprising up to about 3 (such as about any of 1, 2, or 3) amino acid substitutions, a CDR2 comprising the amino acid sequence of FISNGGEEVS (SEQ ID NO:10) or a variant thereof comprising up to about 3 (such as about any of 1, 2, or 3) amino acid substitutions, and a CDR3 comprising the amino acid sequence of RSTDVSPGLSSWWTYEYDV (SEQ ID NO:11) or a variant thereof comprising up to about 3 (such as about any of 1, 2, or 3) amino acid substitutions. In some embodiments, the sdAb moiety comprises a CDR1 comprising the amino acid sequence of GENFERYDMS (SEQ ID NO:7), a CDR2 comprising the amino acid sequence of RLNSFGRSTY (SEQ ID NO:9), and a CDR3 comprising the amino acid sequence of RSTDVSPGLSSWWTYEYDV (SEQ ID NO:11); or a CDR1 comprising the amino acid sequence of GFTFGAYDMV (SEQ ID NO:8), a CDR2 comprising the amino acid sequence of FISNGGEEVS (SEQ ID NO:10), and a CDR3 comprising the amino acid sequence of RSTDVSPGLSSWWTYEYDV (SEQ ID NO:11).

In some embodiments, the sdAb moiety comprises a VHH domain comprising the amino acid sequence of SEQ ID NO:22, or a variant thereof comprising a CDR1 comprising the amino acid sequence of GENFERYDMS (SEQ ID NO:7), a CDR2 comprising the amino acid sequence of RLNSFGRSTY (SEQ ID NO:9), and a CDR3 comprising the amino acid sequence of RSTDVSPGLSSWWTYEYDV (SEQ ID NO:11) and having at least about 90% or at least about 95% sequence identity to SEQ ID NO:22. In some embodiments, the sdAb moiety comprises a VHH domain comprising the amino acid sequence of SEQ ID NO:24, or a variant thereof comprising a CDR1 comprising the amino acid sequence of GENFERYDMS (SEQ ID NO:7), a CDR2 comprising the amino acid sequence of RLNSFGRSTY (SEQ ID NO: 9), and a CDR3 comprising the amino acid sequence of RSTDVSPGLSSWWTYEYDV (SEQ ID NO:11) and having at least about 90% or at least about 95% sequence identity to SEQ ID NO: 24. In some embodiments, the sdAb domain comprises a VHH domain comprising the amino acid sequence of SEQ ID NO:26, or a variant thereof comprising a CDR1 comprising the amino acid sequence of GFTFGAYDMV (SEQ ID NO:8), a CDR2 comprising the amino acid sequence of FISNGGEEVS (SEQ ID NO:10), and a CDR3 comprising the amino acid sequence of RSTDVSPGLSSWWTYEYDV (SEQ ID NO:11) and having at least about 90% or at least about 95% sequence identity to SEQ ID NO:26.

In some embodiments the sdAb moiety comprises an FR1, an FR2, an FR3, and/or an FR4. In some embodiments the sdAb moiety comprises an FR1, an FR2, an FR3, and an FR4.

In some embodiments, the sdAb moiety comprises an FR1 comprising the amino acid sequence of EX¹⁴QLVESGGX¹⁵LVQPGGSLRLSCX¹⁶AS (SEQ ID NO:3), wherein:
X¹⁴ is V or A;
X¹⁵ is G or A; and
X¹⁶ is E or A,
   or a variant thereof comprising up to about 3 (such as about any of 1, 2, or 3) amino acid substitutions. In some versions, the FR1 comprises the sequence of EVOLVESGGGLVQPGGSLRLSCEAS (SEQ ID NO:12) or a variant thereof at least 80%, 85%, 90%, 95%, 96%, 97%, 98%, or 99% thereto. In some versions, the FRI comprises the sequence of EAQLVESGGGLVQPGGSLRLSCEAS (SEQ ID NO:13) or a variant thereof at least 80%, 85%, 90%, 95%, 96%, 97%, 98%, or 99% thereto. In some versions, the FR1 comprises the sequence of EVQLVESGGALVQPGGSLRLSCAAS (SEQ ID NO:14) or a variant thereof at least 80%, 85%, 90%, 95%, 96%, 97%, 98%, or 99% thereto.

In some embodiments, the sdAb moiety comprises an FR2 comprising the amino acid sequence of WVRX¹⁷AX¹⁸GKGPEWX¹⁹S (SEQ ID NO:4), wherein:
X¹⁷ is Q or H;
X¹⁸ is P or T; and
X¹⁹ is V or I,
   or a variant thereof comprising up to about 3 (such as about any of 1, 2, or 3) amino acid substitutions. In some versions, the FR2 comprises the sequence of WVRQAPGKGPEWVS (SEQ ID NO:15) or a variant thereof at least 80%, 85%, 90%, 95%, 96%, 97%, 98%, or 99% thereto. In some versions, the FR2 comprises the sequence of WVRHATGKGPEWIS (SEQ ID NO: 16) or a variant thereof at least 80%, 85%, 90%, 95%, 96%, 97%, 98%, or 99% thereto.

In some versions, the sdAb moiety comprises an FR3 comprising comprises the amino acid sequence of YX²⁰X²¹SVKX²²RFTX²³SRDX²⁴AX²⁵X²⁶X²⁷X²⁸YLQMNNLKPEDX²⁹X³⁰VYYCA (SEQ ID NO: 5), wherein:
X²⁰ is L or A;
X²¹ is D or S;
X²² is G or D;
X²³ is V or I;
X²⁴ is N or T;
X²⁵ is Q or K;
X²⁶ is N or S;
X²⁷ is M or T;
X²⁸ is L or F;
X²⁹ is T or A; and
X³⁰ is A or G,
   or a variant thereof comprising up to about 3 (such as about any of 1, 2, or 3) amino acid substitutions. In some versions, the FR3 comprises the sequence of YLDSVKGRFTVSRDNAQNMLYLQMNNLKPEDTAVYYCA (SEQ ID NO:17) or a variant thereof at least 80%, 85%, 90%, 95%, 96%, 97%, 98%, or 99% thereto. In some versions, the FR3 the sequence comprises of YLDSVKGRFTVSRDTAKSTFYLQMNNLKPEDAGVYYCA (SEQ ID NO:18) or a variant thereof at least 80%, 85%, 90%, 95%, 96%, 97%, 98%, or 99% thereto. In some versions, the FR3 comprises the sequence of YASSVKDRFTISRDNAQNMLYLQMNNLKPEDTAVYYCA (SEQ ID NO:19) or a variant thereof at least 80%, 85%, 90%, 95%, 96%, 97%, 98%, or 99% thereto.

In some versions, the sdAb moiety comprises an FR4 comprising the amino acid sequence of WGQGTX³¹VTVSS (SEQ ID NO:6), wherein X³¹ is Q or L, or a variant thereof comprising up to about 3 (such as about any of 1, 2, or 3) amino acid substitutions. In some versions, the FR4 comprises the sequence of WGQGTQVTVSS (SEQ ID NO:20) or a variant thereof at least 80%, 85%, 90%, 95%, 96%, 97%, 98%, or 99% thereto. In some versions, the FR4 comprises the sequence of WGQGTLVTVSS (SEQ ID NO:21) or a variant thereof at least 80%, 85%, 90%, 95%, 96%, 97%, 98%, or 99% thereto.

The anti-MET sdAb moiety may comprise one or more “hallmark residues” in one or more of the FR sequences. In some embodiments, the anti-MET sdAb moiety may comprise a VHH domain comprising the amino acid sequence of any one of the following: a-1) the amino acid residue at position 37 is selected from the group consisting of F, Y, L, I, and V (such as Y or such as F); a-2) the amino acid residue at position 44 is selected from the group consisting of A, G, E, D, G, Q, R, S, and L (such as G, E, or Q); a-3) the amino acid residue at position 45 is selected from the group consisting of L, R and C (such as L or R); a-4) the amino acid residue at position 103 is selected from the group consisting of G, W, R and S (such as W or R, or such as W); and a-5) the amino acid residue at position 108 is Q; or b-1) the amino acid residue at position 37 is selected from the group consisting of F, Y, L, I, and V (such as Y or such as F); b-2) the amino acid residue at position 44 is selected from the group consisting of E and Q; b-3) the amino acid residue at position 45 is R; b-4) the amino acid residue at position 103 is selected from the group consisting of G, W, R and S (such as W); and b-5) the amino acid residue at position 108 is selected from the group consisting of Q and L (such as Q); wherein the amino acid position is according to Kabat numbering. It should be noted that these “hallmark residues” at amino acid positions 37, 44, 45, 103 and 108 according to Kabat numbering apply to anti-MET sdAb moieties of natural VHH sequences, and can be substituted during humanization. For example, Q at amino acid position 108 according to Kabat numbering can be optionally humanized to L. Other humanized substitutions will be clear to those skilled in the art. For example, potentially useful humanizing substitutions can be determined by comparing the FR sequences of a naturally occurring VHH with the corresponding FR sequences of one or more closely related human V_{H}, then introducing one or more of such potentially useful humanizing substitutions into said VHH using methods known in the art (also as described herein). The resulting humanized VHH sequences can be tested for their MET binding affinity, for stability, for ease and level of expression, and/or for other desired properties. Possible residue substitutions may also come from an antibody V_{H} domain wherein the V_{H}/V_{L} interface comprises one or more highly charged amino acid residues. The anti-MET sdAb moiety described herein can be partially or fully humanized.

In some embodiments, there is provided an anti-MET sdAb moiety comprising a VHH domain comprising the amino acid sequence of any one of SEQ ID NOs: 22, 24, and 26, or a variant thereof having at least about 80% (such as at least about any of 80%, 85%, 90%, 91%, 92%, 93%, 94%, 95%, 96%, 97%, 98%, or 99%) sequence identify to any one of SEQ ID NOs: 22, 24, and 26. In some embodiments, there is provided an anti-MET sdAb moiety comprising a VHH domain comprising the amino acid sequence of any one of SEQ ID NOS: 22, 24, and 26, or a variant thereof comprising up to about 3 (such as about any of 1, 2, or 3) amino acid substitutions in the VHH domain. In some embodiments, the anti-MET sdAb moiety comprising the VHH domain comprising the amino acid sequence of any one of SEQ ID NOs: 22, 24, and 26 or the variant thereof comprises amino acid substitutions in CDRs, such as the CDR1, and/or the CDR2, and/or the CDR3 of any one of SEQ ID NOs: 22, 24, and 26. In some embodiments, the anti-MET sdAb moiety comprising the VHH domain comprising the amino acid sequence of any one of SEQ ID NOs: 22, 24, and 26 or the variant thereof comprises the CDR1, CDR2, and CDR3 of any one of SEQ ID NOs: 22, 24, and 26, and the amino acid substitutions are in FRs, such as the FR1, and/or the FR2, and/or the FR3, and/or the FR4 of any one of SEQ ID NOs: 22, 24, and 26.

Constructs Comprising the Anti-MET sdAb Moiety

The anti-MET construct comprising the anti-MET sdAb moiety can be of any possible format.

In some embodiments, the anti-MET construct comprising the anti-MET sdAb moiety may further comprise additional polypeptide sequences, such as one or more antibody moieties, or Fc fragment of immunoglobulin. Such additional polypeptide sequences may or may not change or otherwise influence the (biological) properties of the sdAb, and may or may not add further functionality to the sdAb described herein. In some embodiments, the additional polypeptide sequences confer one or more desired properties or functionalities to the sdAb of the present invention. In some embodiments, the anti-MET construct is a chimeric antigen receptor (CAR) comprising an extracellular antigen binding domain comprising one or more anti-MET sdAb moieties described herein.

In some embodiments, the additional polypeptide sequences may be a second antibody moiety (such as sdAb, scFv, full-length antibody) that specifically recognizes a second antigen. In some embodiments, the second antigen is not MET. In some embodiments, the second antigen is EGFR. In some embodiments, the second antibody moiety specifically recognizes the same epitope on MET as the anti-MET sdAb described herein. In some embodiments, the second antibody moiety specifically recognizes a different epitope on MET as the anti-MET sdAb described herein.

In some embodiments, the additional polypeptide sequences may increase the antibody construct half-life, solubility, or absorption, reduce immunogenicity or toxicity, eliminate or attenuate undesirable side effects, and/or confer other advantageous properties to and/or reduce undesired properties of the anti-MET construct of the invention, compared to the anti-MET sdAb described herein per se. Some non-limiting examples of such additional polypeptide sequences are serum proteins, such as human serum albumin (see for example WO 00/27435) or haptenic molecules (for example haptens that are recognized by circulating antibodies, see for example WO 98/22141). It was shown that linking fragments of immunoglobulins (such as V_{H} domains) to serum albumin or fragments thereof may increase antibody half-life (see e.g. WO 00/27435 and WO 01/077137). Thus, in some embodiments, the anti-MET construct of the present invention may comprise an anti-MET sdAb moiety described herein linked to serum albumin (or to a suitable fragment thereof), optionally via a suitable linker (such as peptide linker). In some embodiments, the anti-MET sdAb moiety described herein can be linked to a fragment of serum albumin at least comprising serum albumin domain III. (see PCT/EP2007/002817).

### Heavy Chain-Only Antibody (HCAb)

In some embodiments, anti-MET sdAb moiety described herein can be linked to one or more (preferably human) C_{H}2 and/or C_{H}3 domains, optionally via a linker sequence, to increase its half-life in vivo.

Thus in some embodiments, the anti-MET construct is an HCAb (hereinafter referred to as “anti-MET HCAb”) comprising an anti-MET sdAb moiety described herein fused to an Fc fragment of an immunoglobulin, such as IgA, IgD, IgE, IgG, and IgM. In some embodiments, the anti-MET HCAb comprises an Fc sequence of IgG, such as any of IgG1, IgG2, IgG3, or IgG4. In some embodiments, the Fc fragment is a human Fc. In some embodiments, the Fc fragment is a human IgG1 Fc. In some embodiments, the anti-MET HCAb is monomeric. In some embodiments, the anti-MET HCAb is dimeric. In some embodiments, the anti-MET sdAb moiety and the Fc fragment are optionally connected by a peptide linker.

In some embodiments, there is provided an anti-MET HCAb comprising an sdAb moiety of the invention fused to an Fc fragment of an immunoglobulin. In some embodiments, the Fc fragment is a human IgG1 Fc. An exemplary IgG1 Fc is:

(SEQ ID NO: 32)
EPKSSDKTHTCPPCPAPELLGGPSVFLFPPKPKDTLMISRTPEVT
CVVVDVSHEDPEVKFNWYVDGVEVHNAKTKPREEQYNSTYRVVSV
LTVLHQDWLNGKEYKCKVSNKALPAPIEKTISKAKGQPREPQVYT
LPPSRDELTKNQVSLTCLVKGFYPSDIAVEWESNGQPENNYKTTP
PVLDSDGSFFLYSKLTVDKSRWQQGNVFSCSVMHEALHNHYTQKS
LSLSPGK

In some embodiments, the anti-MET HCAb is monomeric. In some embodiments, the anti-MET HCAb is dimeric. In some embodiments, the anti-MET sdAb moiety and the Fc fragment are optionally connected by a peptide linker. In some embodiments, the anti-MET sdAb moiety is camelid, chimeric, human, partially humanized, or fully humanized.

In some embodiments, there is provided an anti-MET HCAb comprising the amino acid sequence of SEQ ID NO:28, or a variant at least 80%, 85%, 90%, 95%, 99% identical thereto.

Multivalent and/or Multispecific Antibodies

In some embodiments, the anti-MET construct comprises an anti-MET sdAb moiety described herein fused to one or more other antibody moiety (such as an antibody moiety that specifically recognizes MET or another antigen). The one or more other antibody moiety can be of any antibody or antibody fragment format, such as a multispecific sdAb (such as bispecific sdAb), a full-length antibody, a Fab, a Fab′, a (Fab′)2, an Fv, a single chain Fv (scFv), an scFv-scFv, a minibody, a diabody, or a sdAb. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046. For a review of multispecific antibodies, see Weidle et al., Cancer Genomics Proteomics, 10 (1): 1-18, 2013; Geering and Fussenegger, Trends Biotechnol., 33 (2): 65-79, 2015; Stamova et al., Antibodies, 1 (2): 172-198, 2012. Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90:6444-6448 (1993). Triabodies and tetrabodies are also described in Hudson et al., Nat. Med. 9:129-134 (2003). Antibody fragments can be made by various techniques, including but not limited to proteolytic digestion of an intact antibody as well as production by recombinant host cells (e.g. E. coli or phage), as described herein. In some embodiments, the one or more other antibody moiety is antibody mimetics, which are small engineered proteins comprising antigen-binding domains reminiscent of antibodies (Geering and Fussenegger, Trends Biotechnol., 33 (2): 65-79, 2015). These molecules are derived from existing human scaffold proteins and comprise a single polypeptide. Exemplary antibody mimetics that can be comprised within the anti-MET construct described herein can be, but are not limited to, a designed ankyrin repeat protein (DARPin; comprising 3-5 fully synthetic ankyrin repeats flanked by N- and C-terminal Cap domains), an avidity multimer (avimer; a high-affinity protein comprising multiple A domains, each domain with low affinity for a target), or an Anticalin (based on the scaffold of lipocalins, with four accessible loops, the sequence of each can be randomized).

Techniques for making multispecific antibodies include, but are not limited to, recombinant co-expression of two immunoglobulin heavy chain-light chain pairs having different specificities (see Milstein and Cuello, Nature 305:537 (1983)), WO 93/08829, and Traunecker et al., EMBO J. 10:3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229:81 (1985)); using leucine zippers to produce bi-specific antibodies (see, e.g., Kostelny et al., J. Immunol., 148 (5): 1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147:60 (1991); and creating polypeptides comprising tandem single-domain antibodies (see, e.g., U.S. patent application No. 20110028695; and Conrath et al. J. Biol. Chem., 2001; 276 (10): 7346-50). Engineered antibodies with three or more functional antigen binding sites, including “Octopus antibodies,” are also included herein (see, e.g., US 2006/0025576A1).

In some embodiments, the anti-MET construct comprises a first anti-MET sdAb moiety of the invention described herein fused to a second anti-MET sdAb moiety of the invention. The first and second anti-MET sdAb moieties can include any anti-MET sdAb moieties described herein. The first and second anti-MET sdAb moieties can be fused via a peptide linker.

In some embodiments, the anti-MET construct comprising an anti-MET sdAb moiety and one or more other antibody moiety is monospecific. In some embodiments, the anti-MET construct comprising an anti-MET sdAb moiety and one or more other antibody moiety is multispecific (such as bispecific). Multispecific molecules are molecules that have binding specificities for at least two different antigens or epitopes (e.g., bispecific antibodies have binding specificities for two antigens or epitopes). Multispecific molecules with more than two valencies and/or specificities are also contemplated. For example, trispecific antibodies can be prepared. Tutt et al. J. Immunol. 147:60 (1991). It is to be appreciated that one of skill in the art could select appropriate features of individual multispecific molecules described herein to combine with one another to form a multi-specific anti-MET molecule of the invention.

In some embodiments, the anti-MET construct is multivalent but monospecific, i.e., the anti-MET construct comprises an anti-MET sdAb moiety described herein and at least a second antibody moiety specifically recognizing the same MET epitope as the anti-MET sdAb moiety. In some embodiments, the one or more antibody moiety specifically recognizing the same MET epitope as the anti-MET sdAb moiety described herein may comprise the same CDRs and/or the same VHH amino acid sequence as the anti-MET sdAb moiety. For example, the anti-MET construct may comprise two or more anti-MET sdAb moieties described herein, wherein the two or more anti-MET sdAb moieties are the same. In some embodiments, the anti-MET sdAb moieties are optionally connected by peptide linker(s).

In some embodiments, the anti-MET construct is multivalent and multispecific, i.e., the anti-MET construct comprises an anti-MET sdAb moiety described herein and at least a second antibody moiety specifically recognizing a second antigen other than MET, or a different MET epitope recognized by the anti-MET sdAb moiety. In some embodiments, the second antibody moiety is a sdAb. In some embodiments, the second antibody moiety specifically recognizes human serum albumin (HSA). In some embodiments, the sdAb moiety specifically recognizing MET is N terminal or C terminal to the second antibody moiety. In some embodiments, the anti-MET construct is trivalent and bispecific. In some embodiments, the anti-MET construct comprises two anti-MET sdAbs described herein and a second antibody moiety (such as an anti-HSA sdAb), wherein the second antibody moiety is in between the two anti-MET sdAbs. In some embodiments, the antibody moieties are optionally connected by peptide linker(s).

In some embodiments, the anti-MET construct comprises an anti-MET sdAb moiety of the invention described herein fused to an anti-EGFR antibody moiety. See, e.g., Cho et al. 2023 (Cho BC, Simi A, Sabari J, Vijayaraghavan S, Moores S, Spira A. Amivantamab, an Epidermal Growth Factor Receptor (EGFR) and Mesenchymal-epithelial Transition Factor (MET) Bispecific Antibody, Designed to Enable Multiple Mechanisms of Action and Broad Clinical Applications. Clin Lung Cancer. 2023 March; 24 (2): 89-97) for an exemplary anti-EGFR antibody suitable for this purpose.

### Peptide Linkers

In some embodiments, the two or more antibody moieties within the anti-MET construct can be optionally connected by a peptide linker. The length, the degree of flexibility and/or other properties of the peptide linker(s) used in the anti-MET construct may have some influence on properties, including but not limited to the affinity, specificity or avidity for one or more particular antigens or epitopes. For example, longer peptide linkers may be selected to ensure that two adjacent domains do not sterically interfere with one another. In some embodiment, a peptide linker comprises flexible residues (such as glycine and serine) so that the adjacent domains are free to move relative to each other. For example, a glycine-serine doublet can be a suitable peptide linker.

The peptide linker can be of any suitable length. In some embodiments, the peptide linker is at least about any of 1, 2, 3, 4, 5, 6, 7, 8, 9, 10, 11, 12, 13, 14, 15, 16, 17, 18, 19, 20, 25, 30, 35, 40, 50, 75, 100 or more amino acids long. In some embodiments, the peptide linker is no more than about any of 100, 75, 50, 40, 35, 30, 25, 20, 19, 18, 17, 16, 15, 14, 13, 12, 11, 10, 9, 8, 7, 6, 5 or fewer amino acids long. In some embodiments, the length of the peptide linker is any of about 1 amino acid to about 10 amino acids, about 1 amino acid to about 20 amino acids, about 1 amino acid to about 30 amino acids, about 5 amino acids to about 15 amino acids, about 10 amino acids to about 25 amino acids, about 5 amino acids to about 30 amino acids, about 10 amino acids to about 30 amino acids long, about 30 amino acids to about 50 amino acids, about 50 amino acids to about 100 amino acids, or about 1 amino acid to about 100 amino acids.

The peptide linker may have a naturally occurring sequence, or a non-naturally occurring sequence. For example, a sequence derived from the hinge region of heavy chain only antibodies may be used as the linker. See, for example, WO1996/34103. In some embodiments, the peptide linker is a mutated human IgG1 hinge (see SEQ ID NO:445 of U.S. Pat. No. 11,673,954). In some embodiments, the peptide linker is a flexible linker. Exemplary flexible linkers include glycine polymers (G)ₙ, glycine-serine polymers (including, for example, (GS)ₙ, (GSGGS (SEQ ID NO:33))ₙ, (GGGS (SEQ ID NO:34))ₙ, and (GGGGS (SEQ ID NO:35))ₙ, where n is an integer of at least one, such as 1, 2, 3, 4, 5, 6, 7, 8, 9, 10, 15, 20, 25 or more, and/or, optionally, up to 10, 15, 20, 25, 30, 35, or more), glycine-alanine polymers, alanine-serine polymers, and other flexible linkers known in the art. Exemplary peptide linkers can include peptide sequences such as GGGGSGGGS (SEQ ID NO:36), GGGGSGGGGSGGGGS (SEQ ID NO:37), GPGGP (SEQ ID NO:38), AALVGPGGQGGGGSGGGGSGGGGSGGGGSGGGGSMA (SEQ ID NO:39), EPKSSDKTHTSPPSP (SEQ ID NO:40), and GPGGQGTGPGGS (SEQ ID NO:41). Other suitable peptide linkers are provided in Klein J S, Jiang S, Galimidi R P, Keeffe J R, Bjorkman P J. Design and characterization of structured protein linkers with differing flexibilities. Protein Eng Des Sel. 2014 October; 27 (10): 325-30.

### Anti-MET Antibody Variants

In some embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleic acid sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### a) Substitution, Insertion, Deletion and Variants

In some embodiments, antibody variants having one or more amino acid substitutions are provided. Sites of interest for substitutional mutagenesis include the HVRs and FRs. Conservative substitutions are shown in Table 2 under the heading of “Preferred substitutions.” More substantial changes are provided in Table 2 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 2
Amino acid substitutions.

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) Hydrophobic: Norleucine, Met, Ala, Val, Leu, Ile;
(2) Neutral hydrophilic: Cys, Ser, Thr, Asn, Gln;
(3) Acidic: Asp, Glu;
(4) Basic: His, Lys, Arg;
(5) Residues that influence chain orientation: Gly, Pro;
(6) Aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001)) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In some embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or CDRs. In some embodiments of the variant VHH sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

### b) Glycosylation Variants

In some embodiments, an anti-MET construct provided herein is altered to increase or decrease the extent to which the construct is glycosylated. Addition or deletion of glycosylation sites to an antibody may be conveniently accomplished by altering the amino acid sequence such that one or more glycosylation sites is created or removed.

Where the anti-MET construct comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an anti-MET construct of the present application may be made in order to create antibody variants with certain improved properties.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about +3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87:614 (2004). Examples of cell lines capable of producing defucosylated antibodies include Lec13 CHO cells deficient in protein fucosylation (Ripka et al. Arch. Biochem. Biophys. 249:533-545 (1986); US Patent Application No. US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87:614 (2004); Kanda, Y. et al., Biotechnol. Bioeng., 94 (4): 680-688 (2006); and WO2003/085107). Anti-MET construct variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### c) Fc Region Variants

In some embodiments, one or more amino acid modifications may be introduced into the Fc region of the anti-MET construct provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In some embodiments, the present application contemplates an anti-MET construct variant that possesses some but not all effector functions, which make it a desirable candidate for applications in which the half-life of the anti-MET construct in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (Cell Technology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). Clq binding assays may also be carried out to confirm that the antibody is unable to bind Clq and hence lacks CDC activity. See, e.g., Clq and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18 (12): 1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9 (2): 6591-6604 (2001)).

In some embodiments, an anti-MET construct variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) Clq binding and/or Complement Dependent Cytotoxicity (CDC), e.g., as described in U.S. Pat. No. 6,194,551, WO 99/51642, and Idusogie et al. J. Immunol. 164:4178-4184 (2000).

In some embodiments, there is provided an anti-MET construct (e.g., a HCAb) variant comprising a variant Fc region comprising one or more amino acid substitutions which increase half-life and/or improve binding to the neonatal Fc receptor (FcRn). Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

Anti-MET constructs (such as HCAb or anti-MET sdAb fused to a full-length antibody) comprising any of the Fc variants described herein, or combinations thereof, are contemplated.

### d) Cysteine Engineered Antibody Variants

In some embodiments, it may be desirable to create cysteine engineered anti-MET constructs, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In some embodiments, any one or more of the following residues may be substituted with cysteine: A118 (EU numbering) of the heavy chain; and S400 (EU numbering) of the heavy chain Fc region. Cysteine engineered anti-MET constructs may be generated as described, e.g., in U.S. Pat. No. 7,521,541.

### e) Antibody Derivatives

In some embodiments, an anti-MET construct provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly (n-vinyl pyrrolidone) polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In some embodiments, conjugates of an anti-MET construct and nonproteinaceous moiety that may be selectively heated by exposure to radiation are provided. In some embodiments, the nonproteinaceous moiety is a carbon nanotube (Kam et al., Proc. Natl. Acad. Sci. USA 102:11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

In some embodiments, an anti-MET construct provided herein may be further modified to contain one or more biologically active protein, polypeptides or fragments thereof. “Bioactive” or “biologically active” as used herein means showing biological activity in the body to carry out a specific function. For example, it may mean the combination with a particular biomolecule such as protein, DNA, etc., and then promotion or inhibition of the activity of such biomolecule. In some embodiments, the bioactive protein or fragments thereof have immunostimulatory/immunoregulatory, membrane transport, or enzymatic activities.

In some embodiments, the bioactive protein or fragments thereof that can be fused with the anti-MET construct described herein is a ligand, such as lymphokines and cellular factors which interact with specific cellular receptor. Lymphokines are low molecular weight proteins which are secreted by T cells when antigens or lectins stimulate T cell growth. Examples of lymphokines include, but are not limited to, interferon-α, interferon-γ, interleukin-1 (IL-1), interleukin-2 (IL-2), interleukin-3 (IL-3), tumor necrosis factor (TNF), a colony stimulating factor (e.g. CSF-1, G-CSF or GM-CSF), chemotaxins, macrophage migration inhibitory factor (MIF), macrophage-activating factor (MAF), NK cell activating factor, T cell replacing factor, leukocyte-inhibitory factor (LIF), lymphotoxins, osteoclast-activating factor (OAF), soluble immune response suppressor (SIRS), growth-stimulating factor, monocyte growth factor, etc. Cellular factors which may be incorporated into the anti-MET fusion proteins of the invention include but are not limited to tumor necrosis factor α (TNFα), interferons (IFNs), and nerve growth factor (NGF), etc.

### Labels

The anti-MET constructs of the invention can be labeled. The labels can assist in the identification or detection of the construct. Labels for this purpose are well-known in the art and include elements such as fluorophores, enzymes, and isotopic labels, such as radiolabels (e.g., radionuclides). Various labels, such as radiolabels, methods of labeling, and methods of using radiolabeled agents for therapy and screening are described in U.S. Pat. No. 11,298,433, which is incorporated herein by reference in its entirety.

### Cytotoxic Agent Conjugates

The anti-MET constructs of the invention can be conjugated to a cytotoxic agent. See, e.g., Thomas et al. 2016 (Thomas A, Teicher B A, Hassan R. Antibody-drug conjugates for cancer therapy. Lancet Oncol. 2016 June; 17 (6): e254-e262. doi: 10.1016/S1470-2045 (16) 30030-4) for exemplary cytotoxic agents and exemplary chemical linkers.

### Pharmaceutical Compositions

Further provided by the present application are pharmaceutical compositions comprising any one of the anti-MET constructs comprising a sdAb specifically recognizing MET as described herein, and optionally a pharmaceutically acceptable carrier. Pharmaceutical compositions can be prepared by mixing an anti-MET construct described herein having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions.

The pharmaceutical composition is preferably to be stable, in which the anti-MET construct comprising anti-MET sdAb described here essentially retains its physical and chemical stability and integrity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10:29-90 (1993). Stability can be measured at a selected temperature for a selected time period. For rapid screening, the formulation may be kept at 40° C. for 2 weeks to 1 month, at which time stability is measured. Where the formulation is to be stored at 2-8° C., generally the formulation should be stable at 30° C. or 40° C. for at least 1 month, and/or stable at 2-8° C. for at least 2 years. Where the formulation is to be stored at 30° C., generally the formulation should be stable for at least 2 years at 30° C., and/or stable at 40° C. for at least 6 months. For example, the extent of aggregation during storage can be used as an indicator of protein stability. In some embodiments, the stable formulation of anti-MET construct described herein may comprise less than about 10% (preferably less than about 5%) of the anti-MET construct present as an aggregate in the formulation.

Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers (e.g. sodium chloride), stabilizers, metal complexes (e.g. Zn-protein complexes); chelating agents such as EDTA and/or non-ionic surfactants.

Examples of physiologically acceptable carriers include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugars such as sucrose, mannitol, trehalose or sorbitol; salt-forming counterions such as sodium; metal complexes (e.g. Zn-protein complexes); and/or nonionic surfactants such as TWEEN™, polyethylene glycol (PEG), and PLURONICS™ or polyethylene glycol (PEG).

Buffers are used to control the pH in a range which optimizes the therapeutic effectiveness, especially if stability is pH dependent. Buffers are preferably present at concentrations ranging from about 50 mM to about 250 mM. Suitable buffering agents for use in the present application include both organic and inorganic acids and salts thereof. For example, citrate, phosphate, succinate, tartrate, fumarate, gluconate, oxalate, lactate, acetate. Additionally, buffers may comprise histidine and trimethylamine salts such as Tris.

Preservatives are added to retard microbial growth, and are typically present in a range from 0.2%-1.0% (w/v). The addition of a preservative may, for example, facilitate the production of a multi-use (multiple-dose) formulation. Suitable preservatives for use in the present application include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” are present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intra-molecular interactions. Tonicity agents can be present in any amount between 0.1% to 25% by weight, preferably 1% to 5%, taking into account the relative amounts of the other ingredients. Preferred tonicity agents include polyhydric sugar alcohols, preferably trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, a-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) are present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/ml, preferably about 0.07 mg/ml to about 0.2 mg/ml.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In order for the pharmaceutical compositions to be used for in vivo administration, they must be sterile. The pharmaceutical composition may be rendered sterile by filtration through sterile filtration membranes. The pharmaceutical compositions herein generally are placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intra-arterial, intralesional or intraarticular routes, topical administration, inhalation or by sustained release or extended-release means. In some embodiments, the pharmaceutical composition is administered locally, such as intratumorally.

Sustained-release preparations may be prepared. Suitable examples of sustained-release preparations include semi-permeable matrices of solid hydrophobic polymers containing the antagonist, which matrices are in the form of shaped articles, e.g. films, or microcapsules. Examples of sustained-release matrices include polyesters, hydrogels (for example, poly (2-hydroxyethyl-methacrylate), or poly (vinylalcohol)), polylactides (U.S. Pat. No. 3,773,919), copolymers of L-glutamic acid and. ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The pharmaceutical compositions herein may also contain more than one active compound as necessary for the particular indication being treated, preferably those with complementary activities that do not adversely affect each other. Alternatively, or in addition, the composition may comprise a cytotoxic agent, chemotherapeutic agent, cytokine, immunosuppressive agent, or growth inhibitory agent. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition.

### Methods of Treating MET-Related Diseases

The anti-MET construct comprising sdAb specifically recognizing MET as described herein, and the compositions (such as pharmaceutical compositions) thereof are useful for a variety of applications, such as in diagnosis, molecular assays, and therapy.

One aspect of the invention provides a method of treating a MET-related condition in an individual in need thereof, comprising administering to the individual a therapeutically effective amount of a pharmaceutical composition comprising the anti-MET construct described herein.

“MET-related condition” as used herein refers to any condition involving aberrant MET expression, activity, and/or signaling. The aberrant MET expression and/or signaling can result from MET gene amplification (e.g., duplication, etc.), splicing mutations (e.g., METex14 mutations), or other mutations or conditions.

In some embodiments, the MET-related condition comprises cancer. Accordingly, in some embodiments, there is provided a method of treating cancer comprising administering to the individual a therapeutically effective amount of a pharmaceutical composition comprising an isolated anti-MET construct comprising a single-domain antibody (sdAb) moiety specifically recognizing MET. In some embodiments, the pharmaceutical composition is administered systemically (such as intravenously). In some embodiments, the pharmaceutical composition is administered locally (such as intratumorally). In some embodiments, the method further comprises administering to the individual an additional cancer therapy (such as surgery, radiation, chemotherapy, immunotherapy, hormone therapy, or a combination thereof). In some embodiments, the individual is a human. In some embodiments, the method of treating cancer has one or more of the following biological activities: (1) killing cancer cells (including bystander killing); (2) inhibiting proliferation of cancer cells; (3) inducing immune response in a tumor; (4) reducing tumor size; (5) alleviating one or more symptoms in an individual having cancer; (6) inhibiting tumor metastasis; (7) prolonging survival; (8) prolonging time to cancer progression; and (9) preventing, inhibiting, or reducing the likelihood of the recurrence of a cancer. In some embodiments, the method of killing cancer cells mediated by the pharmaceutical composition described herein can achieve a tumor cell death rate of at least about any of 40%, 50%, 60%, 70%, 80%, 90%, 95%, or more. In some embodiments, the method of killing cancer cells mediated by the pharmaceutical composition described herein can achieve a bystander tumor cell (uninfected by the oncolytic VV) death rate of at least about any of 10%, 20%, 30%, 40%, 50%, 60%, 70%, 80%, 90%, 95%, or more. In some embodiments, the method of reducing tumor size mediated by the pharmaceutical composition described herein can reduce at least about 10% (including for example at least about any of 20%, 30%, 40%, 60%, 70%, 80%, 90%, or 100%) of the tumor size. In some embodiments, the method of inhibiting tumor metastasis mediated by the pharmaceutical composition described herein can inhibit at least about 10% (including for example at least about any of 20%, 30%, 40%, 60%, 70%, 80%, 90%, or 100%) of the metastasis. In some embodiments, the method of prolonging survival of an individual (such as a human) mediated by the pharmaceutical composition described herein can prolongs the survival of the individual by at least any of 1, 2, 3, 4, 5, 6, 7, 8, 9, 10, 11, 12, 18, or 24 months. In some embodiments, the method of prolonging time to cancer progression mediated by the pharmaceutical composition described herein can prolongs the time to cancer progression by at least any of 1, 2, 3, 4, 5, 6, 7, 8, 9, 10, 11, or 12 weeks.

The methods described herein are suitable for treating a variety of cancers, including both solid cancer and liquid cancer. The methods are applicable to cancers of all stages, including early stage cancer, non-metastatic cancer, primary cancer, advanced cancer, locally advanced cancer, metastatic cancer, or cancer in remission. The methods described herein may be used as a first therapy, second therapy, third therapy, or combination therapy with other types of cancer therapies known in the art, such as chemotherapy, surgery, hormone therapy, radiation, gene therapy, immunotherapy (such as T-cell therapy), bone marrow transplantation, stem cell transplantation, targeted therapy, cryotherapy, ultrasound therapy, photodynamic therapy, radio-frequency ablation or the like, in an adjuvant setting or a neoadjuvant setting (i.e., the method may be carried out before the primary/definitive therapy). In some embodiments, the method is used to treat an individual who has previously been treated. In some embodiments, the cancer has been refractory to prior therapy. In some embodiments, the method is used to treat an individual who has not previously been treated.

Exemplary cancers treatable with the methods of the invention include lung cancer (e.g., non-small cell lung cancer (NSCLC)), gastric cancer, colon cancer, heart cancer, neck cancer, breast cancer, melanoma (e.g., metastatic malignant melanoma), renal cancer (e.g. clear cell carcinoma), head cancer, head and neck cancer, prostate cancer (e.g. hormone refractory prostate adenocarcinoma), bone cancer, pancreatic cancer, skin cancer, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, gastrointestinal carcinoid tumor, colorectal cancer, gastrointestinal stromal tumor, Leiomyosarcoma, T-cell lymphoma, environmentally induced cancers including those induced by asbestos, and combinations of said cancers. The present invention is also useful for treatment of metastatic cancers, especially metastatic cancers that express MET. Cancers whose growth may be inhibited using the antibodies of the invention include cancers typically responsive to immunotherapy.

Aberrant MET expression, activity, and/or signaling has been shown to be involved in the resistance of a number of cancers to therapy. Such cancers include ALK-positive cancers, EGFR-positive cancers, ErbB2-postive cancers, FGFR-driven cancers, and BRAF-mutated tumors, among others (Dagogo-Jack I, Yoda S, Lennerz J K, Langenbucher A, Lin J J, Rooney M M, Prutisto-Chang K, Oh A, Adams N A, Yeap B Y, Chin E, Do A, Marble H D, Stevens S E, Digumarthy S R, Saxena A, Nagy R J, Benes C H, Azzoli C G, Lawrence M S, Gainor J F, Shaw A T, Hata A N. MET Alterations Are a Recurring and Actionable Resistance Mechanism in ALK-Positive Lung Cancer. Clin Cancer Res. 2020 Jun. 1; 26 (11): 2535-2545) (Engelman J A, Zejnullahu K, Mitsudomi T, Song Y, Hyland C, Park J O, Lindeman N, Gale C M, Zhao X, Christensen J, Kosaka T, Holmes A J, Rogers A M, Cappuzzo F, Mok T, Lee C, Johnson B E, Cantley L C, Jänne P A. MET amplification leads to gefitinib resistance in lung cancer by activating ERBB3 signaling. Science. 2007 May 18; 316 (5827): 1039-43) (Ko B, He T, Gadgeel S, Halmos B. MET/HGF pathway activation as a paradigm of resistance to targeted therapies. Ann Transl Med. 2017 January; 5 (1): 4). Exemplary therapies include tyrosine kinase inhibitors such as gefitinib and erlotinib. Accordingly, some versions of the invention are directed to treating such cancers.

Dosages and desired drug concentrations of pharmaceutical compositions of the present application may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles laid down by Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

When in vivo administration of the anti-MET construct comprising an anti-MET sdAb moiety described herein are used, normal dosage amounts may vary from about 10 ng/kg up to about 100 mg/kg of mammal body weight or more per day, preferably about 1 mg/kg/day to 10 mg/kg/day, such as about 1-3 mg/kg/day, about 2-4 mg/kg/day, about 3-5 mg/kg/day, about 4-6 mg/kg/day, about 5-7 mg/kg/day, about 6-8 mg/kg/day, about 6-6.5 mg/kg/day, about 6.5-7 mg/kg/day, about 7-9 mg/kg/day, or about 8-10 mg/kg/day, depending upon the route of administration. It is within the scope of the present application that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays.

In some embodiments, the pharmaceutical composition is administered for a single time (e.g. bolus injection). In some embodiments, the pharmaceutical composition is administered for multiple times (such as any of 2, 3, 4, 5, 6, or more times). If multiple administrations, they may be performed by the same or different routes and may take place at the same site or at alternative sites. The pharmaceutical composition may be administered twice per week, 3 times per week, 4 times per week, 5 times per week, daily, daily without break, once per week, weekly without break, once per 2 weeks, once per 3 weeks, once per month, once per 2 months, once per 3 months, once per 4 months, once per 5 months, once per 6 months, once per 7 months, once per 8 months, once per 9 months, once per 10 months, once per 11 months, or once per year. The interval between administrations can be about any one of 24 h to 48 h, 2 days to 3 days, 3 days to 5 days, 5 days to 1 week, 1 week to 2 weeks, 2 weeks to 1 month, 1 month to 2 months, 2 month to 3 months, 3 months to 6 months, or 6 months to a year. Intervals can also be irregular (e.g. following tumor progression). In some embodiments, there is no break in the dosing schedule. In some embodiments, the pharmaceutical composition is administered every 4 days for 4 times. The optimal dosage and treatment regime for a particular patient can readily be determined by one skilled in the art of medicine by monitoring the patient for signs of disease and adjusting the treatment accordingly.

The pharmaceutical compositions of the present application, including but not limited to reconstituted and liquid formulations, are administered to an individual in need of treatment with the anti-MET construct described herein, preferably a human, in accord with known methods, such as intravenous administration as a bolus or by continuous infusion over a period of time, by intramuscular, intraperitoneal, intracerobrospinal, subcutaneous, intravenous (i.v.), intra-articular, intrasynovial, intrathecal, oral, topical, or inhalation routes. A reconstituted formulation can be prepared by dissolving a lyophilized anti-MET construct described herein in a diluent such that the protein is dispersed throughout. Exemplary pharmaceutically acceptable (safe and non-toxic for administration to a human) diluents suitable for use in the present application include, but are not limited to, sterile water, bacteriostatic water for injection (BWFI), a pH buffered solution (e.g. phosphate-buffered saline), sterile saline solution, Ringer's solution or dextrose solution, or aqueous solutions of salts and/or buffers.

In some embodiments, the pharmaceutical compositions are administered to the individual by subcutaneous (i.e. beneath the skin) administration. For such purposes, the pharmaceutical compositions may be injected using a syringe. However, other devices for administration of the pharmaceutical compositions are available such as injection devices; injector pens; auto-injector devices, needleless devices; and subcutaneous patch delivery systems.

In some embodiments, the pharmaceutical compositions are administered to the individual intravenously. In some embodiments, the pharmaceutical composition is administered to an individual by infusion, such as intravenous infusion. Infusion techniques for immunotherapy are known in the art (see, e.g., Rosenberg et al., New Eng. J. of Med. 319:1676 (1988)).

### Methods of Preparation

The anti-MET construct (such as anti-MET single-domain antibodies) described herein may be prepared using any methods known in the art or as described herein. Also see the following examples.

Methods of preparing single-domain antibodies have been described. See, for example, Els Pardon et al, Nature Protocol, 2014; 9 (3): 674. Single-domain antibodies (such as VHHs) may be obtained using methods known in the art such as by immunizing a Camelid species (such as camel or llama) and obtaining hybridomas therefrom, or by cloning a library of single-domain antibodies using molecular biology techniques known in the art and subsequent selection by ELISA with individual clones of unselected libraries or by using phage display.

For recombinant production of the single-domain antibodies, the nucleic acids encoding the single-domain antibodies are isolated and inserted into a replicable vector for further cloning (amplification of the DNA) or for expression. DNA encoding the single-domain antibody is readily isolated and sequenced using conventional procedures (e.g., by using oligonucleotide probes that are capable of binding specifically to genes encoding the heavy and light chains of the antibody). Many vectors are available. The choice of vector depends in part on the host cell to be used. Generally, preferred host cells are of either prokaryotic or eukaryotic (generally mammalian) origin.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### EXAMPLES

### Example 1. Development of an Engineered Single-Domain Antibody for Targeting GMET in Non-Small Cell Lung Cancer

### INCORPORATION BY REFERENCE

Aspects of the following examples along with additional information pertaining to same have been published in Luo et al. 2024 (Luo N Y, Minne R L, Gallant J P, Gunaratne G S, West J L, Javeri S, Robertson A J, Lake E W, Engle J W, Mixdorf J C, Aluicio-Sarduy E, Nickel K P, Hernandez R, Kimple R J, Baschnagel A M, LeBeau A M. Development of an Engineered Single-Domain Antibody for Targeting MET in Non-Small Cell Lung Cancer. Bioconjug Chem. 2024 Mar. 20; 35 (3): 389-399). The Luo et al. 2024 reference is incorporated herein by reference in its entirety.

### Summary

The mesenchymal epithelial transition factor receptor (MET) is a receptor tyrosine kinase that is upregulated or mutated in 5% of non-small cell lung cancer (NSCLC) patients and overexpressed in multiple other cancers. We sought to develop single-domain camelid antibodies with high affinity for MET that could be used to deliver conjugated payloads to MET expressing cancers. From a naïve camelid variable-heavy-heavy (VHH) domain phage display library, we identified VHH clones (1E7, 1A2, and 2G9) that displayed high affinity for human MET and were cross-reactive with MET across multiple species. 1E7 was expressed as a bivalent human Fc fusion protein (1E7-Fc) and was found to selectively bind to EBC-1 (MET amplified) and UW-Lung-21 (MET exon 14 mutated) cell lines by flow cytometry and immunofluorescence imaging. Next, we investigated the ability of labeled 1E7-Fc ([⁸⁹Zr]Zr-1E7-Fc) to detect MET expression in vivo by PET/CT imaging. [⁸⁹Zr]Zr-1E7-Fc demonstrated rapid localization and high tumor uptake in both xenografts with a % ID/g of 6.4 and 5.8 for EBC-1 and UW-Lung-21 at 24 h, respectively. At the 24 h timepoint, clearance from secondary and non-target tissues was also observed. Altogether, our data suggests that the VHH clones and Fc fusions of same (e.g., 1E7-Fc) represent platform technologies that can be employed to both image and treat MET-altered NSCLC.

### Introduction

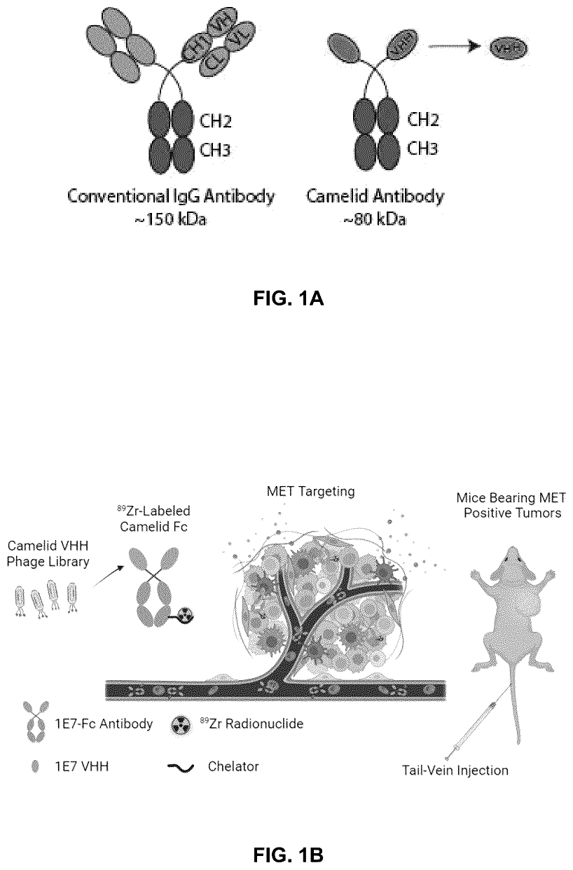

In this study, we document the identification by phage display of a potent VHH domains targeting MET (1E7, 1A2, and 2G9) (FIG. 1B). A VHH-Fc construct, 1E7-Fc, demonstrated high binding affinity to recombinant MET protein and MET-altered NSCLC cell lines through ELISA, biolayer interferometry, flow cytometry, and live cell confocal imaging. In vivo experiments confirmed the stability and ability of VHH-Fc to image MET-altered xenograft models of lung cancer. High uptake was observed in the MET-expression xenograft tumors with low normal tissue uptake, indicating the utility of VHH-Fc as a diagnostic tool and targeted radiotherapy. METexon14 is expressed in non-small cell lung cancer, so targeting the MET protein with an VHH-Fc antibody such as those described herein can be an effective way to image and treat at least this subtype of cancer.

### Results

### Identification and Characterization of Anti-MET VHH Domains

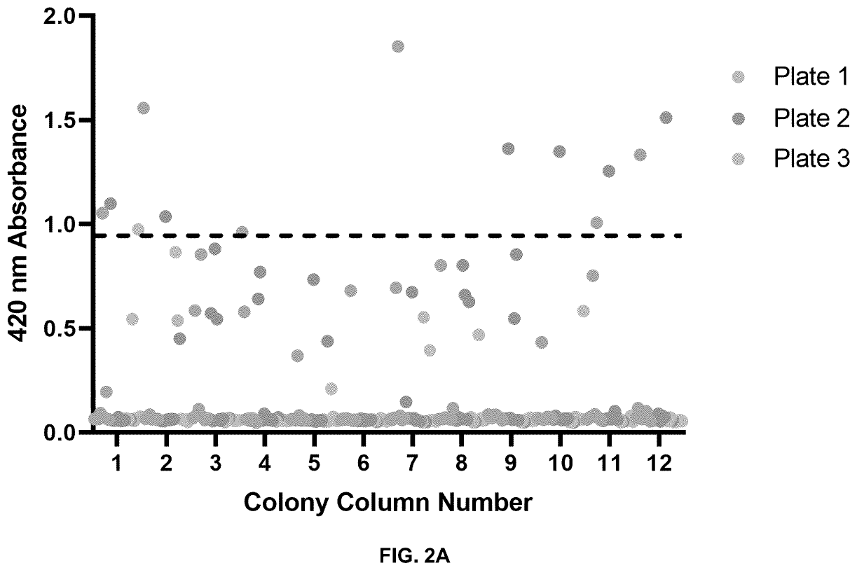

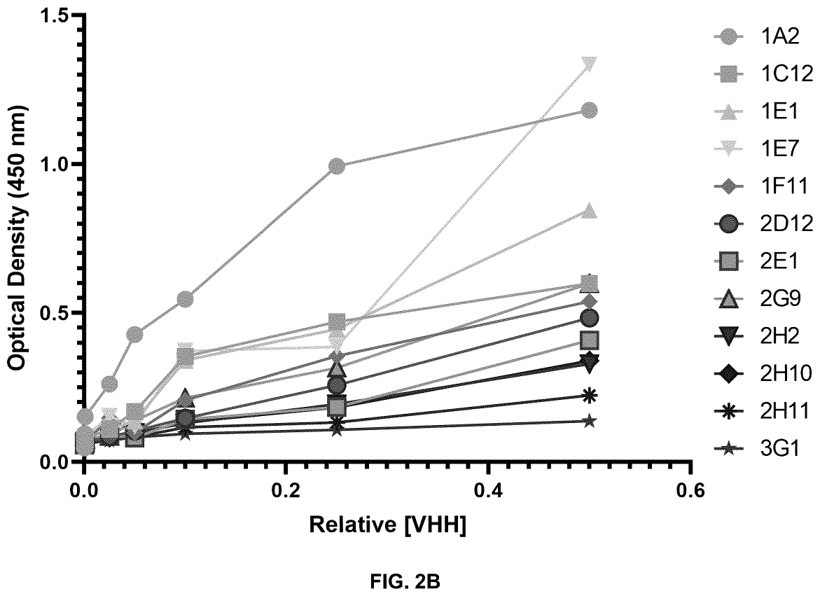

Single-domain constructs specific to MET extracellular domain were identified from a naïve VHH antibody phage display library with a diversity of 7.5×10¹⁰. This library was constructed in-house from B cells isolated from the blood, bone marrow, and spleen tissue of nearly a dozen llamas and alpacas. Library screening was performed against biotinylated human MET immobilized on streptavidin-coated magnetic beads. After four rounds of selection, 384 VHHs were screened against biotinylated human MET by ELISA. A signal threshold of 1.00 was used to identify twelve candidate MET binders by ELISA (FIG. 2A). The twelve unpurified VHHs in culture supernatant were serial diluted and evaluated for a saturating ELISA signal (FIG. 2B). Three (1E7, 1A2, and 2G9) of the twelve clones had unique sequences (Tables 3-6).

TABLE 3
Complementary determining regions
(CDRs) of exemplary VHH sdAbs.

| sdAb | ID | CDR1 | ID | CDR2 | ID | CDR3 |
|---|---|---|---|---|---|---|
| 1E7 | 7 | GENFERYDMS | 9 | RLNSFGRSTY | 11 | RSTDVSPGLS |
| | | | | | | SWWTYEYDV |
| 1A2 | 7 | GENFERYDMS | 9 | RLNSFGRSTY | 11 | RSTDVSPGLS |
| | | | | | | SWWTYEYDV |
| 2G9 | 8 | GFTFGAYDMV | 10 | FISNGGEEVS | 11 | RSTDVSPGLS |
| | | | | | | SWWTYEYDV |

ID: SEQ ID NO

TABLE 4
Framework Regions 1 (FR1) and 2
(FR2) of exemplary VHH sdAbs.

| sdAb | ID | FR1 | ID | FR2 |
|---|---|---|---|---|
| 1E7 | 12 | EVQLVESGGGLVQ | 15 | WVRQAPGKGPEWVS |
| | | PGGSLRLSCEAS | | |
| 1A2 | 13 | EAQLVESGGGLVQ | 15 | WVRQAPGKGPEWVS |
| | | PGGSLRLSCEAS | | |
| 2G9 | 14 | EVQLVESGGALVQ | 16 | WVRHATGKGPEWIS |
| | | PGGSLRLSCAAS | | |

ID: SEQ ID NO

TABLE 5
Framework Regions 3 (FR3) and 4
(FR4) of exemplary VHH sdAbs.

| sdAb | ID | FR3 | ID | FR4 |
|---|---|---|---|---|
| 1E7 | 17 | YLDSVKGRFTVSR | 20 | WGQGTQVTVSS |
| | | DNAQNMLYLQMNN | | |
| | | LKPEDTAVYYCA | | |
| 1A2 | 18 | YLDSVKGRFTVSR | 20 | WGQGTQVTVSS |
| | | DTAKSTFYLQMNN | | |
| | | LKPEDAGVYYCA | | |
| 2G9 | 19 | YASSVKDRFTISR | 21 | WGQGTLVTVSS |
| | | DNAQNMLYLQMNN | | |
| | | LKPEDTAVYYCA | | |

ID: SEQ ID NO

TABLE 6
Exemplary VHH sdAbs.

| sdAb | ID | Sequences |
|---|---|---|
| 1E7 | 22 | EVQLVESGGGLVQPGGSLRLSCEASGENFERYDMSWVRQAPGKGPEWVSRLNSF |
| | | GRSTYYLDSVKGRFTVSRDNAQNMLYLQMNNLKPEDTAVYYCARSTDVSPGLSS |
| | | WWTYEYDVWGQGTQVTVSS |
| 1E7 | 23 | GAGGTCCAGCTGGTGGAGTCTGGGGGAGGCTTGGTGCAGCCTGGGGGTTCTCTG |
| | | AGACTCTCCTGTGAAGCCTCTGGATTCAATTTTGAAAGATATGATATGAGTTGG |
| | | GTCCGCCAGGCTCCAGGAAAGGGGCCCGAGTGGGTGTCACGTCTGAATAGTTTT |
| | | GGACGGAGCACATATTACTTAGATTCTGTGAAGGGCCGATTCACCGTTTCCAGA |
| | | GACAACGCCCAGAACATGCTATATCTGCAAATGAACAACCTGAAACCTGAGGAC |
| | | ACGGCCGTGTATTACTGTGCAAGGTCTACGGACGTCTCACCCGGGCTAAGTAGT |
| | | TGGTGGACATATGAGTATGACGTTTGGGGCCAGGGGACCCAGGTCACCGTCTCC |
| | | AGC |
| 1A2 | 24 | EAQLVESGGGLVQPGGSLRLSCEASGENFERYDMSWVRQAPGKGPEWVSRLNSF |
| | | GRSTYYLDSVKGRFTVSRDTAKSTFYLQMNNLKPEDAGVYYCARSTDVSPGLSS |
| | | WWTYEYDVWGQGTQVTVSS |
| A2 | 25 | GAGGCCCAGCTGGTGGAGTCTGGGGGAGGCTTGGTGCAGCCTGGGGGTTCTCTG |
| | | AGACTCTCCTGTGAAGCCTCTGGATTCAATTTTGAAAGATATGATATGAGTTGG |
| | | GTCCGCCAGGCTCCAGGAAAGGGGCCCGAGTGGGTGTCACGTCTGAATAGTTTT |
| | | GGACGGAGCACATATTACTTAGATTCTGTGAAGGGCCGATTCACCGTTTCCAGA |
| | | GACACGGCCAAGAGTACGTTTTATCTGCAAATGAACAACCTGAAACCTGAGGAT |
| | | GCGGGCGTGTATTACTGTGCAAGGTCTACGGACGTCTCACCCGGGCTAAGTAGT |
| | | TGGTGGACATATGAGTATGACGTTTGGGGCCAGGGGACCCAGGTCACCGTCTCC |
| | | AGC |
| 2G9 | 26 | EVQLVESGGALVQPGGSLRLSCAASGFTFGAYDMVWVRHATGKGPEWISFISNG |
| | | GEEVSYASSVKDRFTISRDNAQNMLYLQMNNLKPEDTAVYYCARSTDVSPGLSS |
| | | WWTYEYDVWGQGTLVTVSS |
| 2G9 | 27 | GAGGTTCAGCTGGTGGAGTCTGGGGGAGCCTTGGTGCAGCCTGGGGGTTCTCTG |
| | | AGACTCTCCTGTGCAGCCTCTGGATTCACCTTCGGAGCCTATGACATGGTCTGG |
| | | GTCCGCCACGCTACTGGAAAGGGGCCCGAATGGATCTCATTCATCTCTAATGGC |
| | | GGTGAAGAGGTATCGTACGCGAGCTCCGTGAAGGACCGATTCACCATCTCCAGA |
| | | GACAATGCCCAGAACATGCTATATCTGCAAATGAACAACCTGAAACCTGAGGAC |
| | | ACGGCCGTGTATTACTGTGCAAGGTCTACGGACGTCTCACCCGGGCTAAGTAGT |
| | | TGGTGGACATATGAGTATGACGTTTGGGGCCAGGGGACCCTGGTCACCGTCTCC |
| | | AGC |
| 1E7-Fc | 28 | METDTLLLWVLLLLAAQPAMAAGSEVQLVESGGGLVQPGGSLRLSCEASGENFE |
| | | RYDMSWVRQAPGKGPEWVSRLNSFGRSTYYLDSVKGRFTVSRDNAQNMLYLQMN |
| | | NLKPEDTAVYYCARSTDVSPGLSSWWTYEYDVWGQGTQVTVSSGPGGQGTGPGG |
| | | SEPKSSDKTHTCPPCPAPELLGGPSVFLFPPKPKDTLMISRTPEVTCVVVDVSH |
| | | EDPEVKFNWYVDGVEVHNAKTKPREEQYNSTYRVVSVLTVLHQDWLNGKEYKCK |
| | | VSNKALPAPIEKTISKAKGQPREPQVYTLPPSRDELTKNQVSLTCLVKGFYPSD |
| | | IAVEWESNGQPENNYKTTPPVLDSDGSFFLYSKLTVDKSRWQQGNVFSCSVMHE |
| | | ALHNHYTQKSLSLSPGK |
| 1E7-Fc | 29 | ATGGAGACTGACACCCTGCTTCTTTGGGTGCTTTTGCTTCTCGCCGCACAACCT |
| | | GCTATGGCCGCTGGATCCGAAGTTCAACTTGTAGAAAGTGGGGGAGGTCTGGTA |
| | | CAACCAGGCGGTTCTCTGAGACTCTCCTGCGAGGCTTCTGGGTTTAACTTCGAA |
| | | CGCTATGACATGAGTTGGGTGCGGCAGGCACCGGGGAAAGGACCGGAATGGGTC |
| | | TCCCGGCTGAATAGCTTTGGGCGCAGCACATATTACCTTGATAGCGTTAAGGGA |
| | | CGCTTCACGGTATCACGAGATAATGCTCAAAACATGTTGTATCTCCAGATGAAT |
| | | AATCTGAAACCGGAAGACACAGCAGTGTACTATTGCGCAAGAAGCACAGACGTT |
| | | TCTCCTGGTCTCTCATCTTGGTGGACCTATGAATATGATGTTTGGGGCCAGGGC |
| | | ACGCAAGTTACGGTCAGTAGTGGGCCGGGCGGCCAGGGTACCGGACCCGGTGGG |
| | | TCTGAACCTAAATCATCTGATAAAACGCACACATGCCCCCCATGTCCTGCGCCA |
| | | GAGCTGCTGGGCGGTCCGTCCGTTTTCCTTTTTCCTCCTAAGCCGAAAGATACG |
| | | TTGATGATTTCAAGGACGCCTGAGGTAACTTGCGTGGTCGTAGATGTAAGCCAT |
| | | GAAGACCCGGAGGTCAAGTTCAACTGGTACGTAGACGGAGTCGAAGTACATAAC |
| | | GCGAAGACGAAGCCACGCGAGGAACAGTACAACAGCACGTATAGGGTAGTCAGC |
| | | GTACTGACCGTCTTGCATCAGGATTGGTTGAACGGTAAGGAGTACAAGTGTAAA |
| | | GTGTCCAATAAGGCCCTTCCCGCGCCGATTGAAAAGACGATTAGCAAAGCCAAA |
| | | GGCCAACCGCGAGAGCCACAGGTTTACACCCTGCCGCCGTCCCGCGATGAACTT |
| | | ACCAAGAACCAGGTCAGTCTGACTTGCCTTGTAAAAGGTTTCTACCCATCTGAC |
| | | ATAGCCGTGGAGTGGGAATCCAATGGGCAACCAGAGAATAACTATAAGACTACT |
| | | CCTCCGGTGCTTGATTCTGACGGGTCTTTTTTCTTGTATAGCAAACTCACGGTT |
| | | GATAAATCTAGGTGGCAACAGGGAAATGTATTTAGTTGTTCTGTAATGCACGAA |
| | | GCCCTTCACAATCACTACACCCAAAAGAGTCTCTCTCTCTCTCCCGGCAAGTGA |

ID: SEQ ID NO

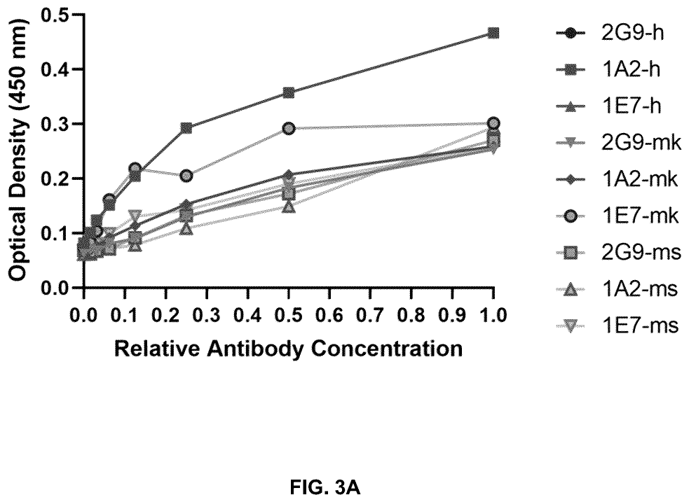

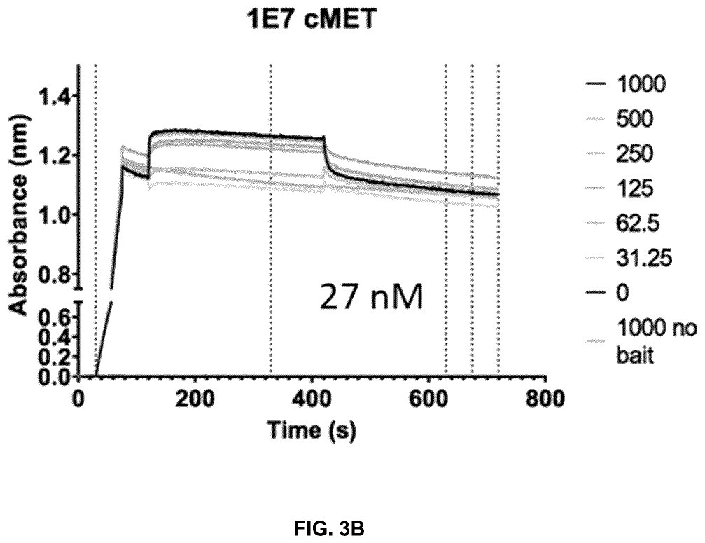

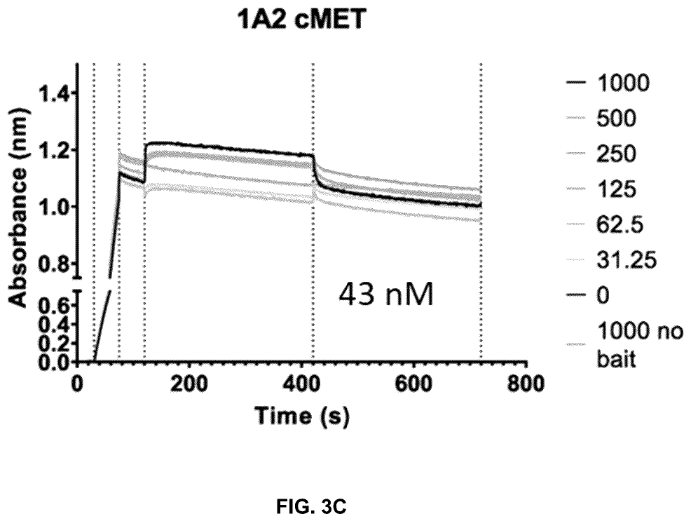

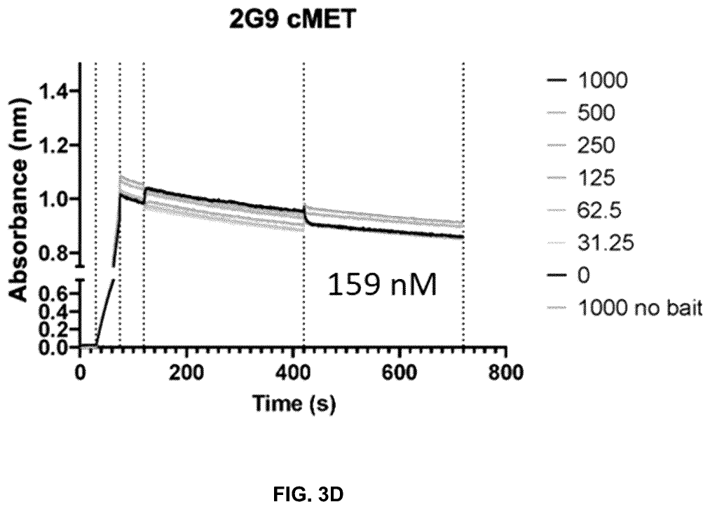

Next, we evaluated the ability of the three unique VHH clones (1E7, 1A2, and 2G9) to bind murine and rhesus monkey MET by performing ELISA counter screens. The VHHs showed modest cross-reactivity with murine and rhesus macaque MET suggesting they recognized common conserved epitopes (FIG. 3A). The three clones were expressed and purified for additional testing to identify a lead VHH. Biolayer interferometry (BLI) was next used to determine the affinity of each VHH domain for MET (FIGS. 3B-D, Table 7). Clone 1E7 demonstrated the highest affinity for MET with a K_{D} of 27 nM (FIG. 3B). When compared to the other clones, 1E7 had the highest kₒₙ and the lowest k_{off}, meaning it possessed faster binding and slower dissociation to MET. Additionally, 1E7 also had the strongest MET signal in the dilution ELISA for human MET. The BLI and dilution ELISA data combined determined that 1E7 was the lead clone to for development into a VHH-Fc targeting MET.

TABLE 7
Bio-layer interferometry values for
the curve fit of 2G9, 1A2, and 1E7.

| Construct | K_{D} | Kₐ | K_{d} | R² |
|---|---|---|---|---|
| 2G9 | 159 | 1.000 × 10⁴ | 1.500 × 10⁻³ | 0.528 |
| 1A2 | 43 | 1.865 × 10⁵ | 2.869 × 10⁻² | 0.984 |
| 1E7 | 27 | 1.445 × 10⁵ | 4.045 × 10⁻² | 0.959 |

### Generation and In Vitro Characterization of Anti-MET VHH-Fc

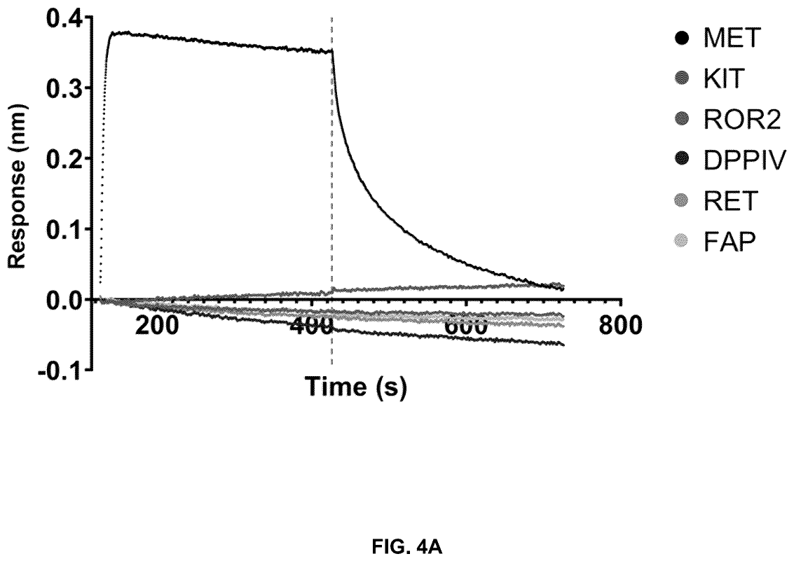

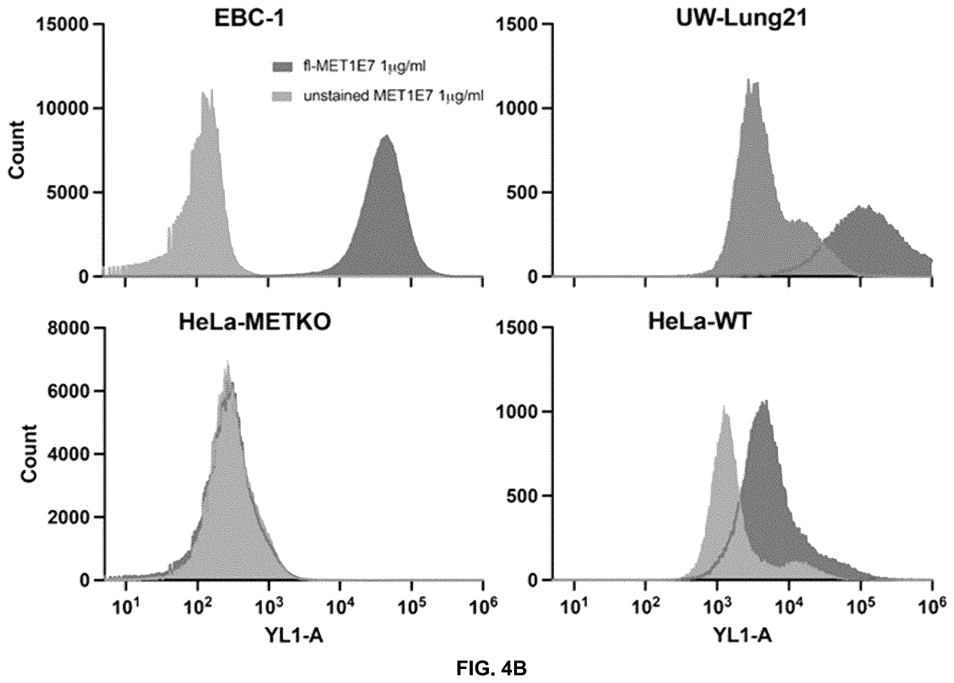

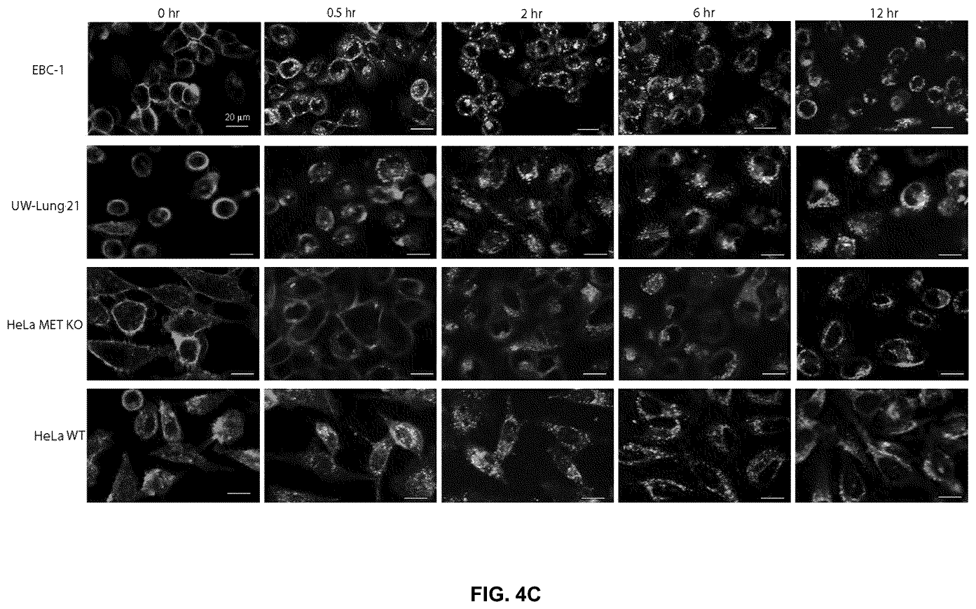

1E7 was cloned into an Fc vector to create a bivalent human Fc construct, 1E7-Fc, with an increased molecular weight of 86.6 kDa. Following expression and purification, the ability of 1E7-Fc to selectively bind to MET-expressing cells as confirmed using multiple in vitro approaches. We confirmed that 1E7-Fc only binds to human MET by performing BLI with human MET and other human surface proteins. The lack of binding to other proteins and binding to MET demonstrates 1E7-Fc binds specifically to MET (FIG. 4A). Two MET-altered NSCLC cell lines, EBC1 and UWLung 21, and two HeLa cell lines, wild type (HeLa WT) and MET knockout (HeLa MET KO), were stained with 1E7-Fc followed by a secondary antibody that was fluorophore labeled for flow cytometry. As indicated by the right shift, 1E7-Fc bound tightly to EBC-1 and UW-Lung 21, moderately to HeLa-WT, and did not bind to HeLa-METKO (FIG. 4B). Live cell immunofluorescent imaging was next performed to determine whether 1E7-Fc was internalized in MET-altered NSCLC cell lines after binding to the MET receptor. The cells were incubated with a green CellMask Plasma Membrane Stain followed by treatment with Alexa Fluor™ 647-labeled 1E7-Fc. At 2 h post-treatment, 1E7-Fc (red) demonstrated high binding to the cell surface (green) with internalization in UWLung 21 and EBC-1, some binding to HeLa WT and no binding to HeLa MET KO. Distinct accumulation within the internal cellular components of MET expressing cell lines is indicative of the internalization of 1E7-Fc (FIG. 4C).

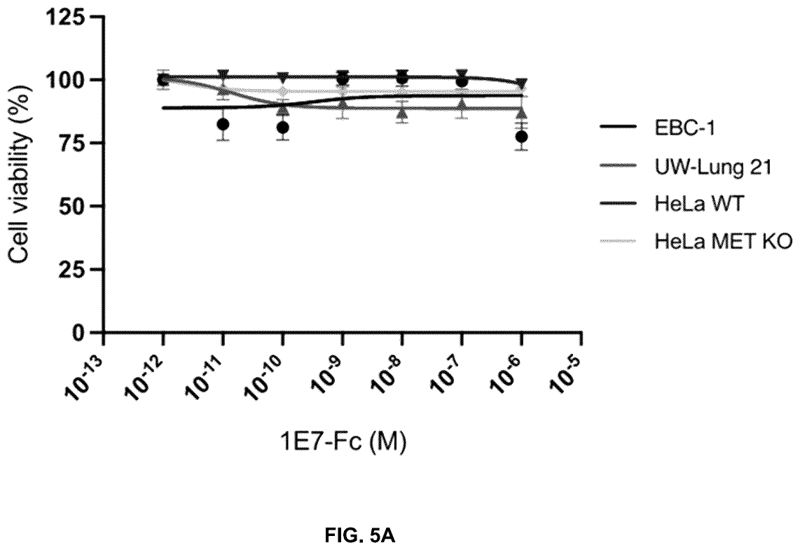

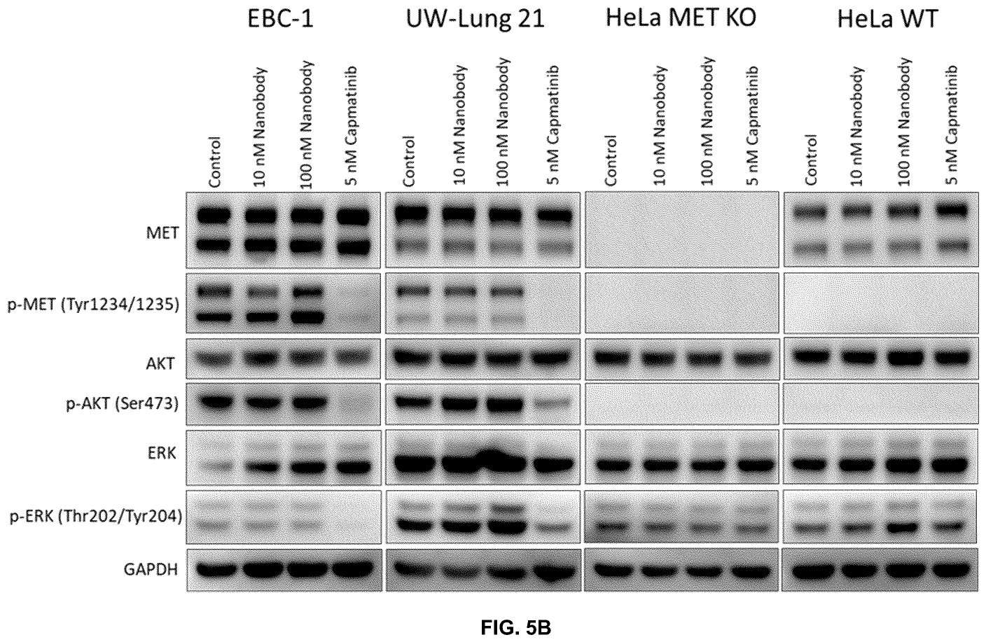

To determine if 1E7-Fc by itself was a therapeutic and inhibited MET signaling, as has been seen with other MET antibodies such as onartuzumab and emibetuzumab (De Mello et al. 2020), we performed a cell proliferation assay and Western blot to query effects of 1E7-Fc on cell behavior and downstream signaling pathways. Cell viability was measured using a panel of the same four cell lines. 1E7-Fc alone had no effect on cell viability even at doses far exceeding binding affinity, suggesting that it was biologically inert (FIG. 5A). Consistent with the lack of cell growth inhibition, 1E7-did not impact the phosphorylation of MET (p-MET) and downstream signaling of AKT and ERK (FIG. 5B). In contrast, treatment with the FDA-approved MET TKI, capmatinib, blocked MET, AKT, and ERK phosphorylation, as anticipated (FIG. 4B). These studies confirmed that 1E7-Fc had no biological effect on METexpressing cells, and it acted as neither an agonist or antagonist.

### In Vivo Imaging of MET by PET/CT

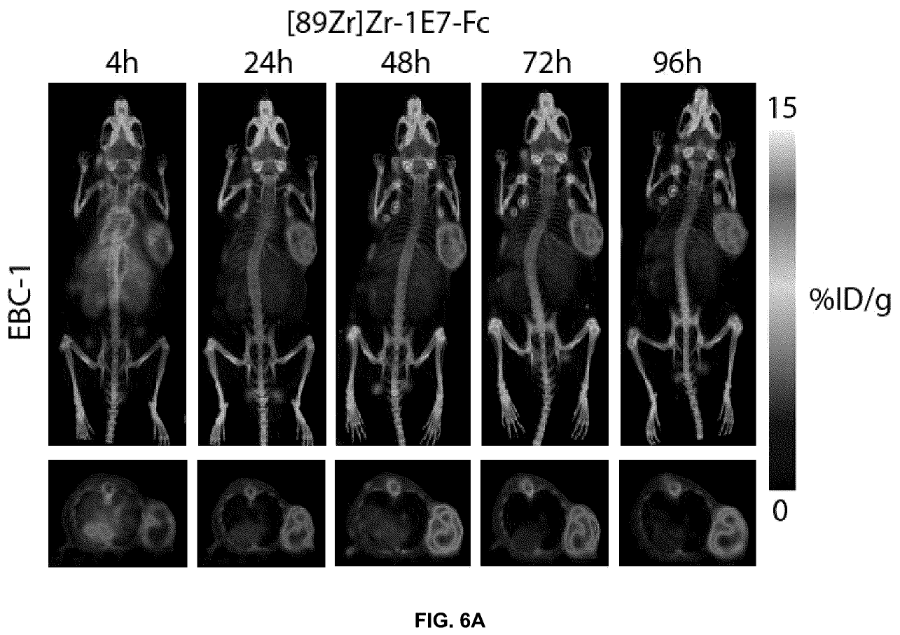

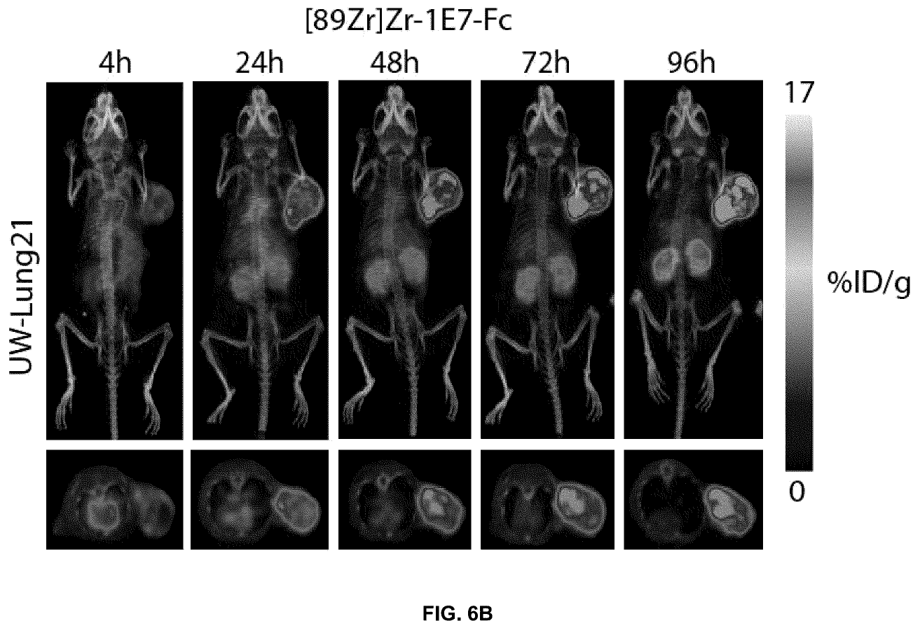

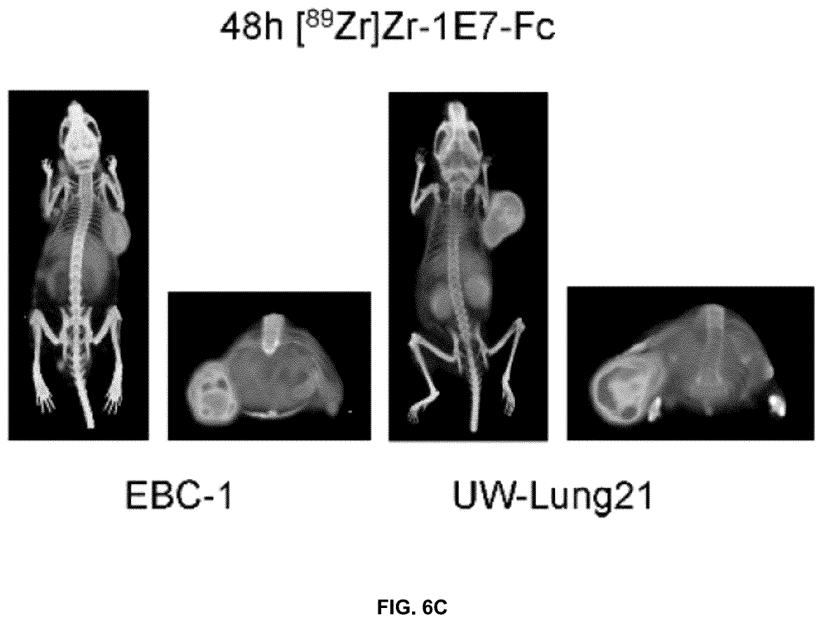

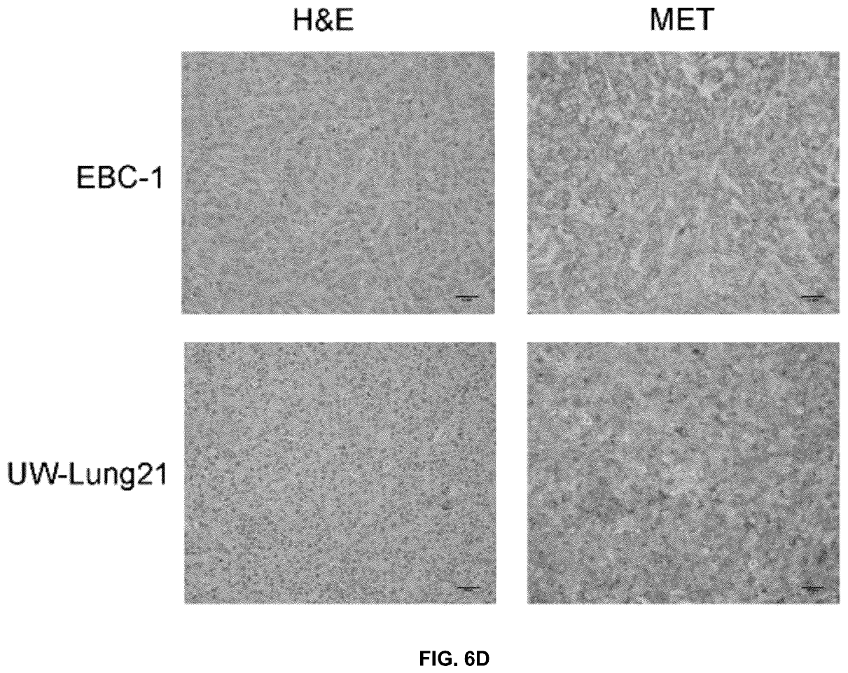

The biodistribution and ability of 1E7-Fc to localize to METexpressing lung cancer models was assessed by PET/CT. 1E7-Fc was radiolabeled with zirconium-89 (89Zr) using standard conditions and injected via tail vein into mice bearing EBC-1 and UW-Lung 21 xenografts. The mice were imaged by PET/CT longitudinally at 4, 24, 48, 72, and 96 h postinjection. 2D and 3D PET images were analyzed to illustrate organ distribution of [89Zr]Zr-1E7-Fc (FIGS. 6A and 6B). The PET/CT imaging study documents that, over time, [89Zr]Zr-1E7-Fc accumulates in the tumor and clears from other organs, showing specificity for MET-expressing tissue. Ex vivo confirmation of MET expression in EBC-1 and UW-Lung 21 xenografts is demonstrated in the positive immunohistochemistry staining.

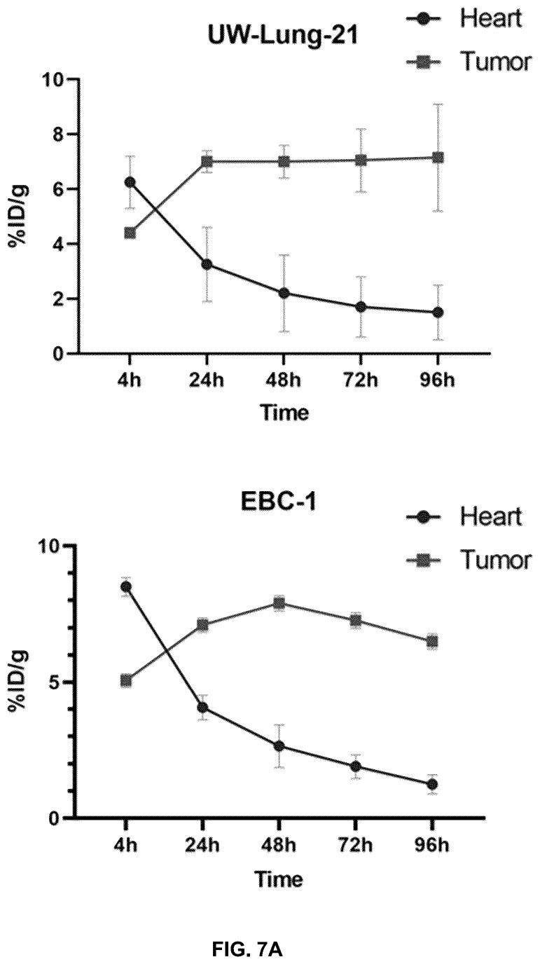

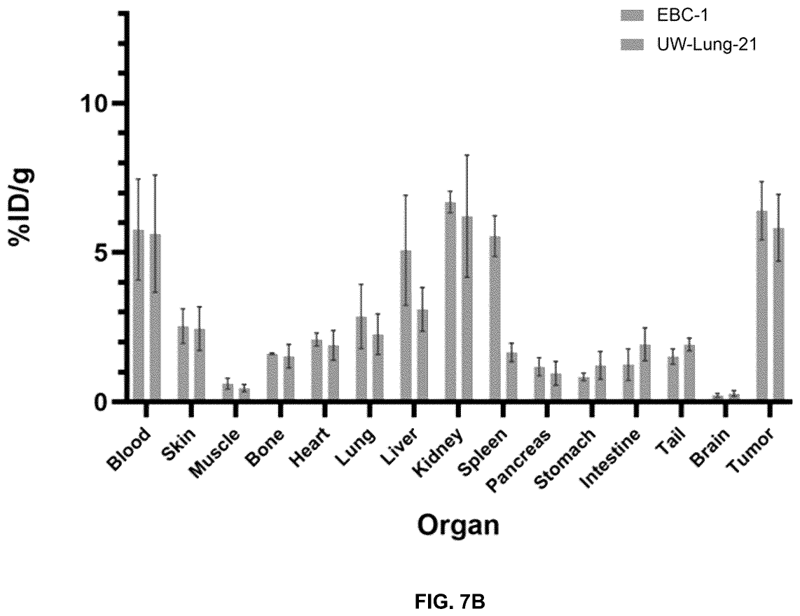

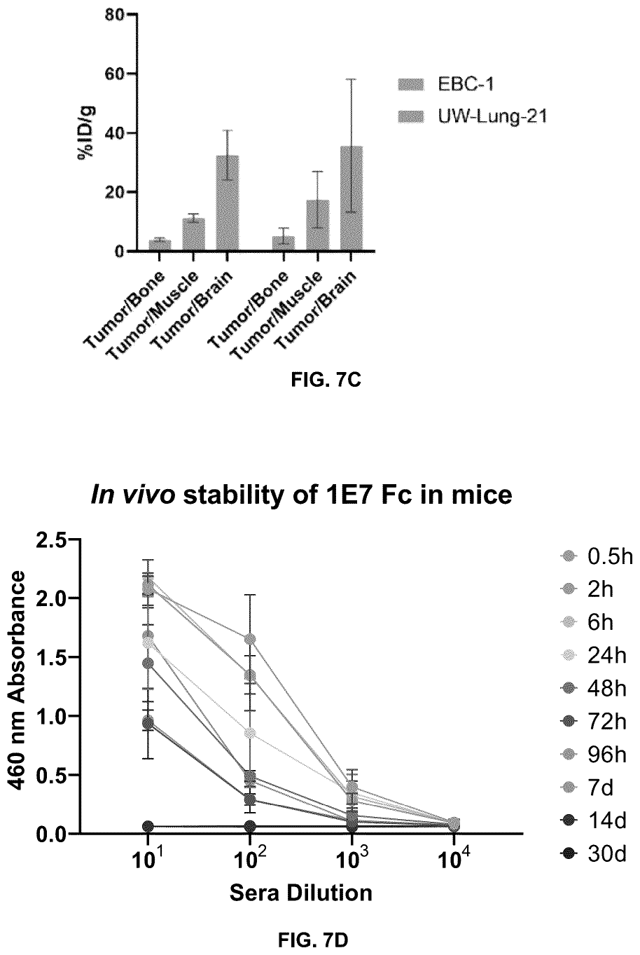

Analysis of the 3D imaging data confirmed high uptake in the MET-positive xenografts at all time points past 4 h. The data were reconstructed using Inveon Research Workplace to draw Regions of Interest (ROI) around selected organs and obtain the mean percentage injected dose per gram (% ID/g) of tissue. The inverse trend between the heart (blood) and tumor over time demonstrated that the administered [89Zr]Zr-1E7-Fc first traveled to the heart and then localized at the tumor (FIG. 7A). The relatively high level of [89Zr]Zr-1E7-Fc in the tumor even at 96 h showed that the antibody is retained in the MET-expressing xenografts (FIG. 7A). As illustrated in FIGS. 6A-6D, the radioactive signal in the tumors of both xenografts nearly reached their zenith at 24 h postinjection. In a separate experiment, xenograft-bearing mice were injected with [89Zr]Zr-1E7-Fc, and the organs were harvested 24 h postinjection to determine the biodistribution of the radiolabeled antibody. The tumors of UW-Lung 21 and EBC-1 have high antibody uptake compared to the muscle, digestive system tissues, brain, and bone. The liver, kidney, and spleen also have relatively high uptake as expected because those organs are responsible for filtering the blood (FIG. 7B). The high tumor-bone, tumor-muscle, and tumor-brain ratios from the biodistribution data further confirm the localization of [89Zr]Zr-1E7-Fc in MET-positive tumors (FIG. 7C).

An in vivo stability assay was performed to characterize the viability of 1E7-Fc to stay in the body for therapy and imaging applications. Nontumor bearing mice were injected with 1E7-Fc via the tail vein, and the blood was collected at different time points across a month. Serum was obtained from the collected blood and assessed for levels of residual 1E7-Fc that binds to human MET on a dilution ELISA. Appreciable binding occurred up to 1 week (FIG. 7D). This suggests that the 1E7-Fc not only can localize in MET-positive tumors but also is stable enough to be potentially utilized for therapy and imaging.

### Discussion

There is a critical unmet need for effective antibody-based therapies that target MET on the surface of transformed cells. Therefore, we developed camelid antibodies that target MET, a proto-oncogene, receptor tyrosine kinase that is upregulated in numerous cancers, including NSCLC. Using a naïve camelid VHH antibody phase display library, we identified three clones (1E7, 1A2, and 2G9). We subsequently engineered one of these clones (1E7) into a bivalent human Fc fusion protein (1E7-Fc). 1E7-Fc demonstrated high specificity to MET-positive cell lines based on flow cytometry and confocal microscopy analysis. These results were validated with in vivo xenografts, demonstrating localization and retention of 1E7-Fc in the tumor out to 96 hr. While 1E7-Fc demonstrated high affinity for the MET receptor, it did not have any inhibitory or cytotoxic effect on our MET-expressing cell lines. 1E7-Fc was internalized by MET-expressing cell lines in vitro. Internalization is a requirement for PET imaging tracers, radiotherapies, and antibody-drug conjugates to be successful and effective.

The most common PET tracer, [¹⁸F]F-FDG, is used to image lung cancer, but is limited in its capabilities to specifically detect METexon14 altered NSCLC. [¹⁸F]F-FDG uptake is also not cancer specific and results in high background radioactivity in brain tissue (Yi et al. 2008). A small-molecule inhibitor of the protease fibroblast activation protein (FAP) labeled with ⁶⁸Ga [⁶⁸Ga]Ga-FAPI-46 has shown greater rates of accurately detecting lung cancer lesions than [¹⁸F]F-FDG, suggesting that there is a possibility for more accurate imaging tracers to better guide treatment decisions (Wang et al. 2022). For MET-specific PET tracers, there have been studies with [⁶⁸Ga]Ga-EMP-100 to target MET in renal cell carcinoma, [¹⁸F]F-AH113804 to detect breast cancer, and 18F labeled MET inhibitor for NSCLC (Arulappu et al. 2016, Mittlmeier et al. 2022, Han et al. 2021. These tracers are at different preclinical and clinical stages. None of these are antibody-based tracers nor are they specific for METexon14 NSCLC. Thus, 1E7-Fc and the other clones described herein represent platform technologies that can exploit the presence of MET on the cell surface to enter the cancer cell to deliver imaging isotopes or multiple therapeutic modalities.

A number of antibodies targeting MET have been developed and investigated for various applications in the clinic. The humanized, one-armed monovalent antihuman MET antibody, onartuzumab, was designed as an antagonist of the MET pathway and performed better than a placebo in a phase II study when combined with erlotinib (Spigel et al. 2017). However, in phase III clinical trials, there was no improved therapeutic effect when combined with erlotinib compared to erlotinib alone Spigel et al. 2013, Elaine et al. 2012, Pool et al. 2017). A PET/CT study with onartuzumab showed promising pre-clinical results of imaging MET when radiolabeled with ⁸⁹Zr. Emibetuzumab, a humanized antibody, was also investigated in combination with erlotinib in patients with Stage IV EGFR mutant NSCLC and in patients with MET-positive metastatic NSCLC who acquired resistance to erlotinib. These Phase II studies showed no reversal of the erlotinib resistance nor significant clinical benefit (Camidge et al. 2022, Scagliotti et al. 2020). Telisotuzumab vedotin (Teliso-V), an antibody-drug conjugate with a microtubule inhibitor warhead, demonstrated antitumor effects during phase I trials. During the phase II trial, Teliso-V had unanticipated toxicity and did not meet the endpoint of the study (Camidge et al. 2021, Waqar et al. 2021). Additional constructs, including a murine antibody and human single-chain variable fragments (scFvs) engineered into bivalent cys-diabodies have been used for imaging MET in preclinical models (Perk et al. 2008, Martinelli et al. 2022, Li et al. 2014). Curiously, radiotherapy studies using MET-targeted antibodies have never been reported in the literature. Overall, the lack of effective antibodies for targeting MET further underscores the need for new biologic scaffolds and constructs.

To our knowledge, we are the first to target the MET receptor with a camelid heavy-chain VHH. Due to the small size and unique characteristics of VHH nanobodies, 1E7-Fc can bind to epitopes inaccessible to the mentioned mouse and human antibodies, allowing it to reach different targets and bind with extreme affinity (Asaadi et al. 2021). When looking at the some of the studies comparing VHH and scFv, camelid VHH is superior than scFv in siRNA delivery, downregulating their target, and neutralizing toxins (Zavoiura et al. 2021, Oliveira et al. 2010, Ben Abderrazek et al. 2011). In regard to molecular imagining, nanobodies have rapid tumor uptake, fast blood clearance, and higher stability than scFc and Fab, making them a more ideal candidate for PET imaging (De Groeve et al. 2010).

The data presented demonstrate that 1E7 and the other VHHs described herein is highly selective antibodies for human MET, and Fc fusions thereof (e.g., 1E7-Fc) can be further developed as a diagnostic or therapeutic agent. Since 1E7-Fc has no cellular toxicity or effect on other parts of the signaling pathway and is stable in vivo, the Fc fusions can be used as imaging agents for MET-driven lung cancers. The internalization of the Fc fusions makes them promising candidates for use in radioimmunotherapy or as antibody-drug conjugates. As a camelid VHH, the Fc fusions such as 1E7-Fc have more stability, smaller size, and possibly stronger binding to c-MET than the murine derived scFvs that are undergoing clinical trials. There are many potential future directions for 1E7-Fc and the other antibodies described herein to image and treat MET exon 14 skipping lung cancer.

### Methods

### Cell Culture

Two human NSCLC cell lines expressing MET, HeLa wild type, and HeLa Met knock-out cell line were used in this study. EBC-1 was derived from squamous cell carcinoma, has high MET amplification (Lutterbach et al. 2007), and was obtained from JCRB Cell Bank (Xenotech, Kansas City, KS). HeLa wild type and HeLa MET KO cell lines were obtained from Abcam (Waltham, MA, Cat #ab255928 and ab265961, respectively). The UW-Lung-21 PDX was derived from a human lung adenocarcinoma brain metastasis harboring a MET exon 14 skipping mutation. The stable UW-Lung 21 cell line was generated as previously described (Baschnagel et al. 2021). Briefly, tumors taken from early UW-Lung-21 Patient-derived xenograft (PDX) passages were dissociated into single cell suspensions and seeded in 6-well plates containing media (RPMI with 10% fetal bovine serum, 1% L-glutamine, 1% penicillin/streptomycin, and 2.5 μg/mL amphotericin B) and incubated at 37° C. in a humidified atmosphere of 5% CO2. Media was changed regularly, and cells were passaged once they reached 70% confluence. Mycoplasma testing and short tandem repeat profiling was performed routinely. All cell lines had their identity confirmed via short tandem repeat profiling analysis (Table 8), were maintained, used mycoplasma free at a lower passage, and were cultured as described in Table 9. Capmatinib was purchased from Selleck Chemicals (Houston, TX).

TABLE 8
STR profiling of cell lines used in this study.

| | UW-Lung 21 | | | EBC-1 |
|---|---|---|---|---|
| | | Genetica: | UW TRIP Lab: | UW TRIP Lab: |
| | P0* | May 3, 2021 | May 24, 2022 | Feb. 8, 2022 |
| FGA | 21, 21.2 | 20, 21, 21.2 | 20, 21, 21.2 | 22, 22 |
| TPOX | 8, 9 | 8, 9 | 8, 9 | 8, 8 |
| D8S1179 | 12, 16 | 11, 12, 13, 16 | 11, 12, 13, 16 | 14, 14 |
| vWA | 14, 16 | 14, 16 | 14, 16 | 16, 17 |
| Amelogenin | X, Y | X, X | X, X | X, X |
| Penta_D | 12, 14 | 10, 12, 13, 14 | 10, 12, 13, 14 | 13, 13 |
| CSF1PO | 10, 12 | 10 | 10, 10 | 10, 10 |
| D16S539 | 12, 12 | 12, 13 | 12, 13 | 9, 9 |
| D7S820 | 10, 11 | 10, 11, 12 | 10, 11, 12 | 10, 11 |
| D13S317 | 10, 10 | 10, 11 | 10, 11 | 12, 12 |
| D5S818 | 11, 12 | 11, 12 | 11, 12 | 11, 11 |
| Penta_E | 8, 15 | 7, 8, 15, 16 | 7, 15, 16 | 16, 16 |
| D18S51 | 15, 21 | 17, 21 | 17, 21 | 12, 17 |
| D21S11 | 29, 30 | 28, 29, 30, | 28, 29, 30, | 29, 30 |
| | | 33.2 | 33.2 | |
| TH01 | 9, 9.3 | 9, 9.3 | 8, 9, 9.3 | 7, 7 |
| D3S1358 | 16, 16 | 16, 17, 18 | 16, 17, 18 | 15, 15 |
| Passage # | P0 | P5 | P15 | P7 |

*Baschnagel A M, Kaushik S, Durmaz A, Goldstein S, Ong I M, Abel L, Clark PA, Gurel Z, Leal T, Buehler D, Iyer G, Scott J G, Kimple R J. Development and characterization of patient-derived xenografts from non-small cell lung cancer brain metastases. Sci Rep. 2021 Jan. 28; 11(1): 2520.

TABLE 9

| Cell | | |
|---|---|---|
| line | Source | Culture condition |
| EBC-1 | JCRB (Japanese | EMEM with 200 mM L-glutamine, |
| | Collection of | 10% FBS, penicillin (100 units/mL), |
| | Research | streptomycin (100 mg/mL) |
| | Bioresources) | |
| | Cell Bank | |
| | Catalog #: | |
| | JCRB0820 | |
| UW- | Established in | ATCC modified RPMI-1640 (catalog #: |
| Lung | Randall Kimple | 30-2001), 10% FBS, penicillin (100 |
| 21 | lab | units/mL), streptomycin (100 mg/mL) |
| HeLa | Abcam | DMEM (high glucose), 10% FBS, |
| wild | (catalog #: | penicillin (100 units/mL), streptomycin |
| type | ab255928) | (100 mg/mL) |
| HeLa | Abcam | DMEM (high glucose), 10% FBS, |
| MET | (catalog #: | penicillin (100 units/mL), streptomycin |
| knockout | ab265961) | (100 mg/mL) |

### Phage Display Biopanning

A camelid VHH phage display library previously constructed by our lab with a size of 7.5×10\hat{ }10 was used to identify single-domain fragments against human MET (Ye et al. 2021). Four rounds of bead panning with different amounts of MET antigen (100 ug, 50 ug, and 12.5 μg for the last two rounds) were performed to enrich for binders with high affinity for MET. Phage bound to MET-covered streptavidin beads (XXX) were eluted with 1 ml 100 mM triethylamine and neutralized with 500 μl 1 M Tris-HCl, pH 7.5. At every round, TG1s were infected with neutralized phage to proceed to the next round, and at rounds three and four, SS320s were also infected for screening and identifying strong binders.

### ELISA

VHH screening: 384 individual clones were selected and produced using 5 mM IPTG induction in a 96-well flat bottom plate. The VHHs released into the supernatant were screened for binding to human cMET by ELISA. MaxiSorp plates (Nunc) were coated with 50 μl of streptavidin (1 μg/mL in PBS, Promega) overnight at 4C. The wells were washed twice with PBS and blocked with 2% BSA for 1h, shaking at room temperature. All proceeding washes were done in PBS and 0.005% Tween20. The wells were washed three times and covered with 50 μl of human cMET (1 μg/mL in PBS) for 1h, shaking at room temperature. Three washes were done, and the supernatant with released VHH was added to the well (with 1% BSA, 0.005% Tween 20). The plates were shaken for 1 h at room temperature. The wells were washed a final three times, and a 1:1000 dilution of HRP Anti-HA-Proxidase tag antibody (Roche) in PBS, 1% BSA and 50 μL Turbo TMB reagent (Pierce). 2.5 M H2SO4 was used to stop the reaction. Absorbance at 450 nm was measured with a microplate reader (Tecan), and clones with high absorbance signals were identified as positive cMET binders and sent for sequencing to identify unique sequences.

Dilution ELISA: MaxiSorp plates were prepared as described above. 50 μL of biotinylated human cMET, murine cMET, or rhesus macaque cMET was added to the wells. 1E7 monomeric VHH was serial diluted from 1 μM to 15.6 nM in PBS, 1% BSA < and 0.005% Tween20 and added to each well. Wells with protein but no 1E7 VHH were used as a negative control. 1E7 binding was detected, as described above.

### VHH Expression and Purification

Unique VHHs with high affinity to human MET were transformed in Shuffle T7 Competent Escherichia coli K12 cells (NEB). Individual colonies were selected and amplified overnight at 37C in 100 mL of Terrific Broth (TB) with 100 μg/mL ampicillin and 0.04% Glycerol. The 100 mL overnight cultures were used to inoculate 2 L of TB with 100 μg/mL ampicillin and 0.04% Glycerol. Protein expression was induced by adding 1 M IPTC and cultured overnight at 25C. Cells were harvested by centrifugation at 5000×g for 15 min, and periplasmic E. coli fraction was extracted by sonication. The supernatant was obtained after centrifugation at 5000×g for 30 min and run through a 5 mL HisTrap column (Cytiva). The column was then loaded on a High-Performance Liquid Chromatography machine to purify the VHH into PBS and 10% Glycerol. Eluted VHH was combined and concentrated using a 10 kDa centrifugal filter (Millipore) for analysis.

### Fc Production and Purification

The 1E7 sequence was cloned into a TGEX human Fc expression vector and transformed into Stellar competent cells (Clontech), and plasmid DNA was purified using PureLink HiPure plasmid maxiprep kits (Thermo Fisher) according to vendor's recommended protocol. Purified plasmids were then transfected into ExpiCHO-S cells using an Expifectamine CHO Transfection Kit (Thermo Fisher) according to the vendor's recommended protocol, using 1 μg of plasmid DNA and 3.2 μl of Expifectamine CHO Reagent per ml of suspension culture. Cells were incubated at 37° C. and 8% CO2 overnight, shaking at 120 rpm. The next day, transfected cells were supplemented with 6 μl of ExpiCHO Enhancer and 240 μl of ExpiCHO Feed per ml of suspension culture, and incubator conditions were changed to 32° C. and 5% CO2. After 11 days, suspension cultures were harvested and centrifuged at 2000xrcf for 10 min at 4° C. The protein-containing supernatant was collected and further clarified by centrifuging at 20,000xrcf for 30 min at 4° C. and passed through a 0.22 μm sterile filter. The 1E7-Fc antibody was captured using protein A and further purified by size exclusion chromatography. A HiTrap Protein A HP column (Cytiva) was equilibrated with 5 column volumes (CVs) of PBS. Clarified ExpiCHO-S supernatant was supplemented with 300 mM NaCl, pH was adjusted to 6.8, and was pumped onto the protein A column at 1CV/min. Unbound protein was washed from the column using 10 CVs of PBS. Antibodies were eluted in 2.5 CVs of 100 mM glycine pH 3.0, followed by 2.5 CVs of PBS. Eluate was immediately neutralized using 2M Tris HCl pH 8.6. Eluates were concentrated and buffer exchanged into PBS using an Amicon stirred chamber with a 30 kDa MWCO ultrafiltration membrane (Millipore). Size exclusion chromatography was performed on an ÄKTApure fast protein liquid chromatography system using a HiLoad 16/600 Superdex 200 PG (Cytiva) column equilibrated with PBS. Samples were loaded and fractionated using a mobile phase of PBS at 0.5 ml/min, chromatograms were obtained by monitoring UV absorbance at 280 nm. Eluted protein fractions contributing to a single peak of UV absorbance corresponding to the theoretical molecular mass of 1E7-Fc were collected and pooled. Eluates were diluted to 1 mg/ml in PBS, dispensed into 0.5 ml aliquots, and flash frozen.

### Biolayer Interferometry (BLI)

Biolayer interferometry was done with the Octet R8 (Sartorius) instrument. Biotinylated human c-MET was captured on SAX (high precision streptavidin) biosensor. 1 to 4 serial dilutions of 1E7-Fc in 1% BSA starting at 1 μM were bound to the biosensor tip with c-MET.

### Live Cell Confocal Microscopy

Cells were seeded into an 8 well chamber coverslip slide (#1.5 thickness) at approximately 20,000 to 40,000 cells per well depending on cell type. After incubating at 37C for 48 hours, cells were stained with a green CellMask™ Plasma Membrane stain (C37608, Thermo Fisher Scientific) in accordance with manufacturer procedure. The stained cells were then placed in a temperature and CO2 controlled Tokei hit Biochamber and set to be imaged every minute for 2 hours using a Nikon Ti2 Spinning Disk Confocal Microscope at an objective of 40x with 488 nm and 547 nm laser settings. At the beginning of the imaging sequence, cells were treated with 1 mM Alexa Fluor™ 647-labeled 1E7-Fc antibody (A20186, Thermo Fisher Scientific) for 10 m. The cell media was then suctioned out and phenol red free DMEM media was added (21-063-029, Thermo Fisher Scientific). Images were processed using Nikon NIS-Elements imaging software and ImageJ.

### Flow Cytometry

Cells were harvested with Trypsin for 5 minutes at 37C and 5% CO2. 1×10\hat{ }6 cells per condition were incubated with 1 ug/mL of 1E7 Fc for 1 h on ice. Cells were washed and stained with 5 μg/mL Goat anti-Human IgG Fc, PE for 1 h on ice. Cells were washed three times and resuspended in flow cytometry staining buffer (eBioscience). Data was collected with the Attune Flow Cytometer (ThermoFischer), and at least 10 000 viable cells were gated and analyzed with FlowJo software.

### Cell Proliferation

Cells were plated in 96-well plates at densities ranging from 3,000 to 6,000 cells per well according to each cell growth rate. Twenty-four hours after-plating, cells were treated with indicated doses of 1E7-Fc or control (PBS) and incubated for 72 h. After 72 h drug treatment, Cell Counting Kit-8 reagent was added (Dojindo Molecular Technologies) and absorbance measured at 450 nm on a SpectraMax i3 plate reader (Molecular Devices). The absorbance of treated wells was normalized to control wells and the half-maximal inhibitory concentration (IC50) values were calculated using GraphPad Prism.

### Western Blot

Cells were seeded in 10 cm dishes and treated with either control (PBS), 10 nM, or 100 nM 1E7-Fc when cells were reached about 75% confluency, and then cells were harvested 4 h after treatment. Five nM Capmatinib was used as a positive control. Cell lysates were generated as previously described (SenthilKumar et al. 2020). The specific antibodies and sources are listed in Table 10.

TABLE 10
Antibodies used in this study.

| Antibody | Source | Catalog # | Company | Dilution | Detection method |
|---|---|---|---|---|---|
| MET | Rabbit | CST 8198 | Cell Signaling | 1:1000 | Western Blot |
| | | | Technology | | |
| phospho-Met | Rabbit | CST 3077 | Cell Signaling | 1:500 | |
| (Tyr1234/1235) | | | Technology | | |
| AKT | Rabbit | CST 2920 | Cell Signaling | 1:1000 | |
| | | | Technology | | |
| phospho-AKT | Rabbit | CST 4060 | Cell Signaling | 1:1000 | |
| (Ser473) | | | Technology | | |
| p44/42 MAPK | Rabbit | CST 4696 | Cell Signaling | 1:1000 | |
| (Erk1/2) | | | Technology | | |
| phospho-p44/42 | Rabbit | CST 4370 | Cell Signaling | 1:500 | |
| MAPK (Erk1/2) | | | Technology | | |
| (Thr202/Try204) | | | | | |
| GAPDH | Rabbit | CST5174 | Cell Signaling | 1:1000 | |
| | | | Technology | | |

### Animal Models

All animal studies were conducted under a protocol approved by the University of Wisconsin Institutional Animal Care and Use Committee. Three-to-four-week-old homozygous Foxn1\hat{ }nu athymic nude female mice were purchased from Envigo for biodistribution and nuclear imaging studies. Xenografts (n=3/cell line) were made by injecting each cell line (1×10\hat{ }6 cells/mL; 100 μL per mouse) in 50% matrigel subcutaneously above the right shoulder blade. Tumors were allowed to grow for five weeks for the nuclear imaging experiment.

### In Vivo Stability of 1E7

Five-week-old BALB/cJ white mice were purchased from Envigo for in vivo stability study. Mice were intravenously injected through the tail vein with 1E7-Fc (100 μg in 100 μl PBS). Mice were euthanized, and whole blood was collected at 10 different time points. Mouse sera were collected by centrifuging the whole blood at 1500×g for 10 minutes. The sera were then used in ELISA to evaluate their binding to human c-MET.

### Bioconjugation and Radiochemistry

1E7 Fc was conjugated to p-SCN-Bn-Deferoxamine (DFO) as described previously (Zeglis et al. 2015). Zirconium-89 [89Zr] was purchased from the University of Wisconsin Medical Physics Department (Madison, WI). [89Zr]Zr-oxalate in 1.0 mol/L oxalic acid was adjusted to pH 7.5 with 1.0 mol/L Na₂CO₃. To radiolabel the 1E7-Fc, the DFO-1E7-Fc conjugate in 0.5 mol/L HEPES (pH 7.5) was added to the neutralized [89Zr]Zr-oxalate solution and incubated at room temperature with rotation for 1 hour. A size-exclusion PD-10 column preequilibrated with PBS buffer was used to purify the labeled 1E7-Fc. This was then evaluated by radioactive TLC for purity using standard methods as described previously (Glumac et al. 2020). Radiolabeled product did not go through size-exclusion HPLC analysis with a radioactive detector.

### Biodistribution

Nanobodies were labeled with [89Zr] and injected into five-to-six-week-old female Foxn1\hat{ }nu athymic nude mice (Envigo) by tail vein injection. Mice (n=3 mice for each tissue per time point) were euthanized at 4, 24, 48, 72, and 96 hours post-injection. Organs and tumors were harvested and evaluated on an automatic gamma-counter (Hidex). The total counts per minute (cpm) of every organ was compared with a standard syringe of known activity and mass. Count data were background- and decay-corrected, and the percentage injected dose per gram (% ID/g) for each tissue sample was calculated by normalization to the total amount of activity injected into each mouse.

### PET/CT Imaging

Images were acquired on Inveon uPET/CT Scanner (Siemens Medical Solutions). Mice (n=3 for experimental and control groups) were injected with [89Zr]Zr-1E7 Fc by tail vein injection. Mice were anesthetized by inhaling 2.5% isoflurane. Images were recorded at 4, 24, 48, and 96 hour time points. PET list mode data were acquired for 80 million counts using a gamma ray energy window of 350-650 KeV and coincidence timing window of 3.428 ns. A CT-based attenuation correction was performed for approximately 10 minutes with 80 kVp, 1 mA, 220 rotation degrees in 120 rotation steps, 250 ms exposure time, and subsequently reconstructed using a Shepp-Logan filter with 210 micron isotropic voxels. Scans were reconstructed using 3-dimensional ordered-subset expectation maximization (2 iterations, 16 subsets) with a maximum a posteriori probability algorithm (OSEM3DMAP). Two-dimension (2D) images and maximum intensity projections (MIPs) were analyzed in Inveon Research Workplace and ROIs were manually drawn and quantified in Inveon Research Workspace.

### Example 2. Evaluation of a Novel MET-Targeting Camelid-Derived Antibody in Head and Neck Cancer

### Summary

In head and neck squamous cell carcinoma (HNSCC), the Mesenchymal Epithelial Transition (MET) receptor drives cancer growth, proliferation, and metastasis. We evaluated a novel single-domain camelid antibody targeting MET, with potential theranostic application in MET-expressing HNSCC using positron emission tomography/computed tomography (PET/CT) imaging. Immunostaining for MET protein was performed on a tissue microarray from 270 patients with HNSCC. The Cancer Genome Atlas (TCGA) database provided a cohort of 486 HNSCC patient tumors, categorized into high and low MET expression groups based on RNA expression and human papillomavirus (HPV) status. Immunohistochemistry (IHC) assessed MET and phosphorylated-MET expression in head and neck cancer cell lines and xenografts. We evaluated the binding affinity and specificity of a novel camelid nanobody fused to a human IgG Fc chain (1E7-Fc) using flow cytometry and immunofluorescence. Proliferation assay measured 1E7-Fc's effects on cell viability. The efficacy and biodistribution of [⁸⁹Zr]Zr-1E7-Fc as a PET imaging agent were investigated in a MET-expressing head and neck xenograft model. MET protein expression was found in 86% of patients, with 14% having high and 53% low MET expression. High MET RNA expression was associated with worse progression-free survival in patients who were HPV-negative and not in patients who were HPV-positive. In vitro, 1E7-Fc showed high binding affinity and specificity to high MET-expressing Detroit 562 cells, but not to low MET-expressing other HNSCC cells and had no cytotoxic effects. [⁸⁹Zr]Zr-1E7-Fc rapidly localized and showed high tumor uptake in Detroit 562 xenografts (8.4% ID/g at 72 h post-injection), with rapid clearance from the circulatory system (2.7 blood-to-tumor radioactivity ratio at 72 h post-injection). Our preclinical data suggest that the camelid antibody MET-1E7-Fc could be a potential theranostic agent for HNSCC. MET-1E7-Fc can be used in patients as an imaging agent and radionuclide therapeutic for MET-driven cancers.

### Introduction

The MET receptor, also known as the c-Met or hepatocyte growth factor receptor (HGFR), is a receptor tyrosine kinase encoded by the MET gene. It plays a key role in cell proliferation, embryogenesis, tissue development, and wound healing (Birchmeier et al. 1998, Birchmeier et al. 2003, Trusolino et al. 2010. In cancer, the receptor activates downstream signaling pathways such as the mitogen-activated protein kinase (MAPK) cascade, phosphatidylinositol-3 kinase (PI3K)/AKT axis, Janus kinase/signal transducer and transcription activator (JAK/STAT3), and nuclear factor kappa B (NF-κB) complex (Ponzetto et al. 1994, Gherardi et al. 2012, Fan et al. 2005, Johnson et al. 2002, Zhang et al. 2002). In head and neck squamous cell carcinoma (HNSCC), increased activation of the HGF/MET signaling pathway results from various aberrations including overexpression of both MET and HGF protein and amplification of the MET gene. In addition, paracrine signaling, overexpression of the ligand, and autocrine loop formation can also lead to sustained MET activation. Overexpression of the MET protein from transcriptional upregulation in the absence of gene amplification is the most frequently observed MET alteration, being present in greater than 80% of HNSCC tumors (Aebersold et al. 2001, Taskin et al. 2004, Kim et al. 2010, Kim et al. 2011, Lim et al. 2012, Baschnagel et al. 2014). MET amplification, on the other hand, has been reported to be seen in approximately 13% of HNSCC (Seiwert et al. 2009), while MET mutations occur in very few HNSCC tumors (Cancer Genome Atlas Network 2015).

Studies have correlated MET expression level with advanced primary tumor stage, involvement of lymph nodes, resistance to standard therapy, and poorer overall survival across subsites of HNSCC (Aebersold et al. 2001, Yücel et al. 2004, Kim et al. 2010, Kim et al. 2011, Lim et al. 2012, Baschnagel et al. 2014, Akervall et al. 2004, Baschnagel et al. 2017). Patients with HNSCC and with high MET expression, as determined by IHC, have worse locoregional control, distant metastasis, disease-free survival, and overall survival compared to patients with low or no MET expression (Baschnagel et al. 2014). After adjusting for site, T-stage, tobacco history, and p16 status, high MET expression continued to predict worse disease-free survival (Baschnagel et al. 2014). In addition, high MET expression is associated with high CD44 expression, p16-negative tumors, and epidermal growth factor receptor (EGFR)-positive tumors (Baschnagel et al. 2017). A multitude of other studies including at least four separate meta-analyses have confirmed overexpression of MET to be associated with higher clinical stage and worse prognosis (Szturz et al. 2017, Kim et al. 2017, Vsiansky et al. 2018, Li et al. 2019).

The abundance of data on aberrant MET signaling in HNSCC and its role in promoting both metastasis and resistance to standard therapies provides a strong rationale for identifying and developing novel agents that target the MET signaling pathway as an anti-cancer strategy. Indeed, multiple preclinical studies have shown anti-tumor success through suppressing MET signaling with monoclonal antibodies or inhibiting the tyrosine kinase domain of the MET protein itself in HNSCC models (Seiwert et al. 2009, Knowles et al. 2009, Sen et al. 2011, Xu et al. 2011, Liu et al. 2011, Stabile et al. 2013, Baschnagel et al. 2015, Xi et al. 2015, Nisa et al. 2020, Lüttich et al. 2021). In the clinic, both small molecules targeting MET and monoclonal antibodies targeting HGF have been evaluated (Seiwert et al. 2013, Kochanny et al. 2020, Bauman et al. 2020). Two tyrosine kinase inhibitors, foretinib and tivantinib, have been studied in advanced HNSCC and unfortunately demonstrated little clinical benefit (Seiwert et al. 2013, Kochanny et al. 2020). However, ficlatuzumab, a humanized anti-HGF immunoglobulin G1 (IgG1) monoclonal antibody, has demonstrated clinical benefit when given with cetuximab in recurrent and metastatic HSNCC refractory to cetuximab, platinum, and immunotherapy (Bauman et al. 2020, Bauman et al. 2023). No anti-MET monoclonal antibodies have been evaluated in patients with HNSCC and no MET-directed therapy has gained regulatory approval in patients with HNSCC.

Previous therapies may have failed because patients were not appropriately selected based on their MET expression levels or because MET is not an oncogenic driver in HNSCC. Molecular imaging may help identify patients who would respond to specific therapies including MET-directed therapies, and antibodies targeting MET can be used to deliver drugs or radionuclides to MET-expressing cancers even in cancers that are not dependent on MET. None of the previous studies used camelid antibody binding domains. Antibodies derived from camelids (llamas, alpacas, camels, etc.) are unique in that they possess immunoglobulins that have single-chain only variable regions for antigen engagement rather than canonical heavy-light chain pairs (Riechmann et al. 1999). The object of this study was to evaluate a novel single-domain potent MET-binding camelid antibody for the use of positron emission tomography (PET) imaging in MET-expressing HNSCC to identify tumors that would benefit from MET-targeted therapies.

### Results

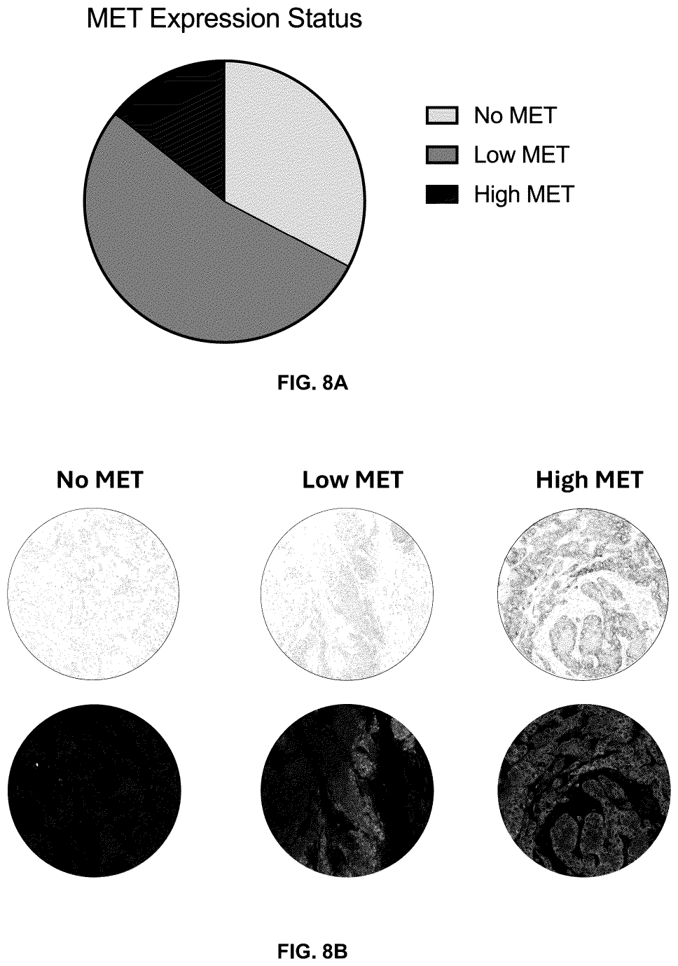

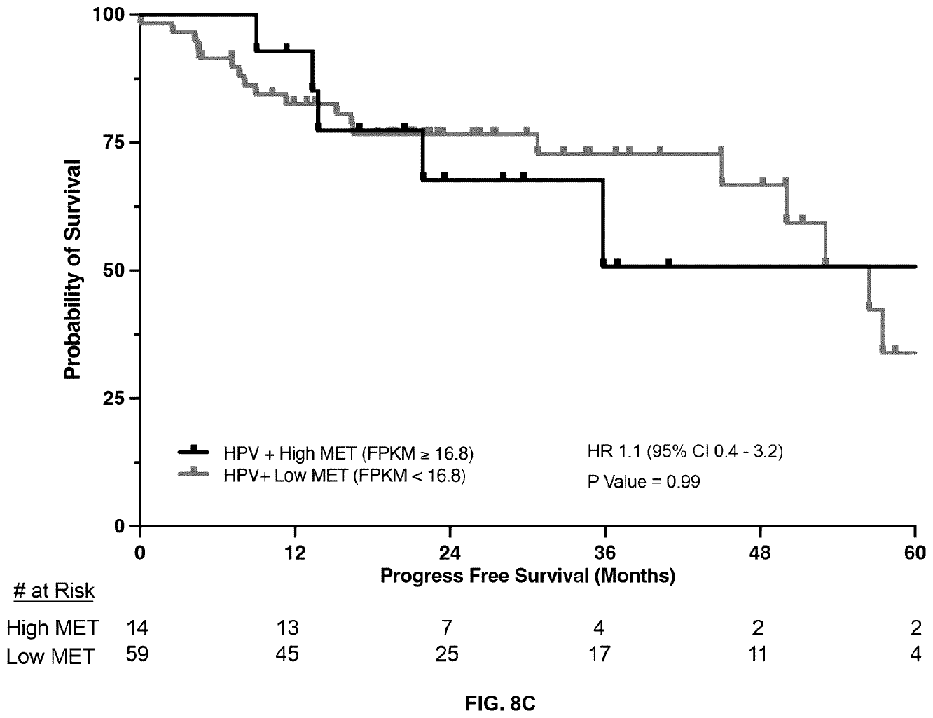

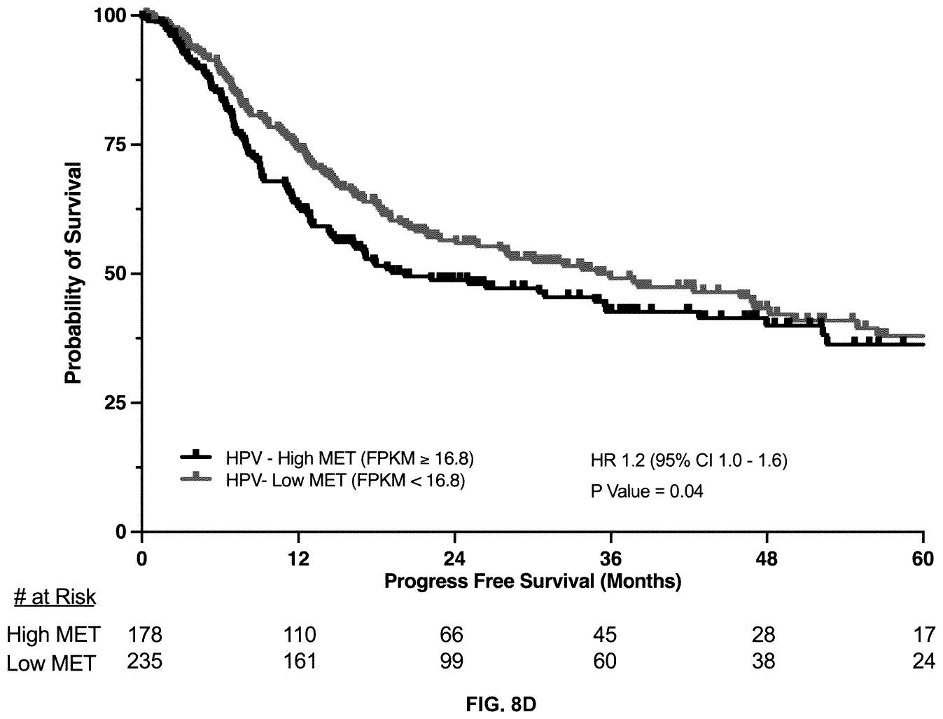

The characteristics of the 203 patients included in the tissue microarray (TMA) analysis are given in Table 11. All patients had oropharyngeal cancer and HPV was positive in 66.5%, negative in 17.7%, and unknown in 15.8% of patients. MET protein was expressed in 85.7% of all tumors including high MET expression in 33%, low MET expression in 52.7% and no MET expression in 14.3% (FIGS. 8A and 8B). There was no association between MET expression and p16 status (Table 11).

TABLE 11
Patient characteristics by MET expression in non-recurrent
University of Wisconsin oropharynx tumors.

| | No MET | | Low MET | | High MET | | |
|---|---|---|---|---|---|---|---|
| Patient Characteristic | Expression | | expression | | expression | | p value |
| No. of patients (%) | 71 | (33.0%) | 102 | (52.7%) | 30 | (14.3%) | |
| Age (y) | | | | | | | |
| Median (range) | 58.6 | (41.8-83.4) | 58.2 | (36.8-88.4) | 59.2 | (37-80.9) | 0.81 |
| Sex | | | | | | | |
| Male | 58 | (81.7%) | 80 | (78.4%) | 28 | (93.3%) | 0.090 |
| Female | 13 | (18.3%) | 22 | (21.6%) | 2 | (6.7%) | |
| Primary Tumor Site | | | | | | | |
| Oropharynx | 27 | (38.0%) | 39 | (38.2%) | 15 | (50%) | 0.66 |
| Base of Tongue | 25 | (35.2%) | 36 | (35.3%) | 5 | (16.7%) | |
| Tonsil | 15 | (21.1%) | 23 | (22.5%) | 6 | (20%) | |
| Soft Palate | 4 | (5.6%) | 4 | (3.9%) | 1 | (3.3%) | |
| p16 | | | | | | | |
| Negative | 14 | (19.7%) | 15 | (14.7%) | 7 | (23.3%) | 0.58 |
| Positive | 44 | (62.0%) | 68 | (66.7%) | 23 | (79.7%) | |
| Unknown | 13 | (18.3%) | 19 | (18.6%) | 0 | (0%) | |
| T classification | | | | | | | |
| T1 | 30 | (42.3%) | 37 | (36.3%) | 12 | (40%) | 0.83 |
| T2 | 23 | (32.4%) | 40 | (39.2%) | 13 | (43.3%) | |
| T3 | 9 | (12.7%) | 12 | (11.8%) | 4 | (13.3%) | |
| T4 | 5 | (7.0%) | 5 | (4.9%) | 0 | (0%) | |
| Unknown | 4 | (5.6%) | 8 | (7.8%) | 1 | (3.3%) | |
| N classification | | | | | | | |
| N0 | 13 | (18.3%) | 12 | (11.8%) | 4 | (13.3%) | 0.50 |
| N1 | 8 | (11.3%) | 15 | (14.7%) | 6 | (20%) | |
| N2 | 42 | (59.2%) | 67 | (65.7%) | 20 | (66.7%) | |
| N3 | 5 | (7.0%) | 5 | (4.9%) | 0 | (0%) | |
| Unknown | 3 | (4.2%) | 3 | (2.9%) | 0 | (0%) | |
| Tobacco use | | | | | | | |
| Yes | 35 | (49.3%) | 67 | (65.7%) | 20 | (66.7%) | 0.070 |
| No | 36 | (50.7%) | 35 | (34.3%) | 10 | (33.3%) | |

Four hundred and eighty-six patients with HNSCC were analyzed from the TCGA database. Mean MET RNA expression measured was 16.8 FPKM (range: 0.3-82.6). Patients were divided into high and low MET expression based on mean FPKM RNA expression. Analysis of patient characteristics revealed patients with high MET expression were more likely to be HPV-negative compared to those with low MET expression (87.2% vs 75.3%, p<0.001) (Table 12). High MET expression correlated with worse PFS in patients with HPV-negative HNSCC (HR: 1.2, 95% CI 1.0-1.6, p=0.04) but not in patients with HPV-positive HNSCC (FIGS. 8C and 8D).

TABLE 12
Patient characteristics by MET RNA expression
in TCGA head and neck cancer cohort.

| | Low MET | | High MET | | |
|---|---|---|---|---|---|
| Patient Characteristic | Expression | | expression | | p value |
| No. of patients (%) | 267 | (54.93%) | 219 | (45.06%) | |
| Age (y) | | | | | |
| Median (range) | 60 | (19, 90) | 62 | (24, 87) | 0.22 |
| Sex | | | | | |
| Male | 198 | (74.2%) | 159 | (72.6%) | 0.77 |
| Female | 69 | (25.8%) | 60 | (27.4%) | |
| Stage | | | | | |
| Stage I | 17 | (6.4%) | 9 | (4.1%) | 0.55 |
| Stage II | 35 | (13.1%) | 36 | (16.4%) | |
| Stage III | 44 | (16.5%) | 35 | (16.0%) | |
| Stage IV | 130 | (48.7%) | 113 | (51.6%) | |
| Unknown | 41 | (15.4%) | 26 | (11.9%) | |
| p16 | | | | | |
| Positive | 58 | (21.7%) | 18 | (8.2%) | <0.001 |
| Negative | 201 | (75.3%) | 191 | (87.2%) | |
| Unknown | 8 | (3.0%) | 10 | (4.6%) | |
| T classification | | | | | |
| T0 | 0 | (0%) | 1 | (0.5%) | 0.55 |
| T1 | 29 | (10.9%) | 18 | (8.2%) | |
| T2 | 73 | (27.3%) | 57 | (26.0%) | |
| T3 | 45 | (16.9%) | 48 | (21.9%) | |
| T4 | 82 | (30.7%) | 73 | (33.3%) | |
| TX | 22 | (8.2%) | 16 | (7.3%) | |
| Unknown | 16 | (6.0%) | 6 | (2.7%) | |
| N classification | | | | | |
| N0 | 85 | (31.8%) | 75 | (34.2%) | 0.75 |
| N1 | 36 | (13.5%) | 28 | (12.8%) | |
| N2 | 82 | (30.7%) | 75 | (34.2%) | |
| N3 | 6 | (2.2%) | 2 | (0.9%) | |
| NX | 41 | (15.3%) | 32 | (14.6%) | |
| Unknown | 17 | (6.3%) | 7 | (3.2%) | |
| Tobacco use | | | | | |
| Yes | 193 | (72.3%) | 171 | (78.1%) | 0.18 |
| No | 67 | (25.1%) | 43 | (19.6%) | |
| Unknown | 7 | (2.6%) | 5 | (2.3%) | |

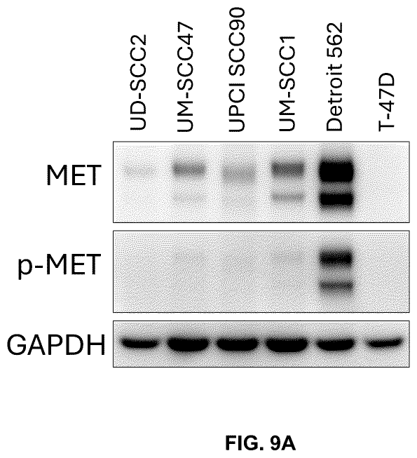

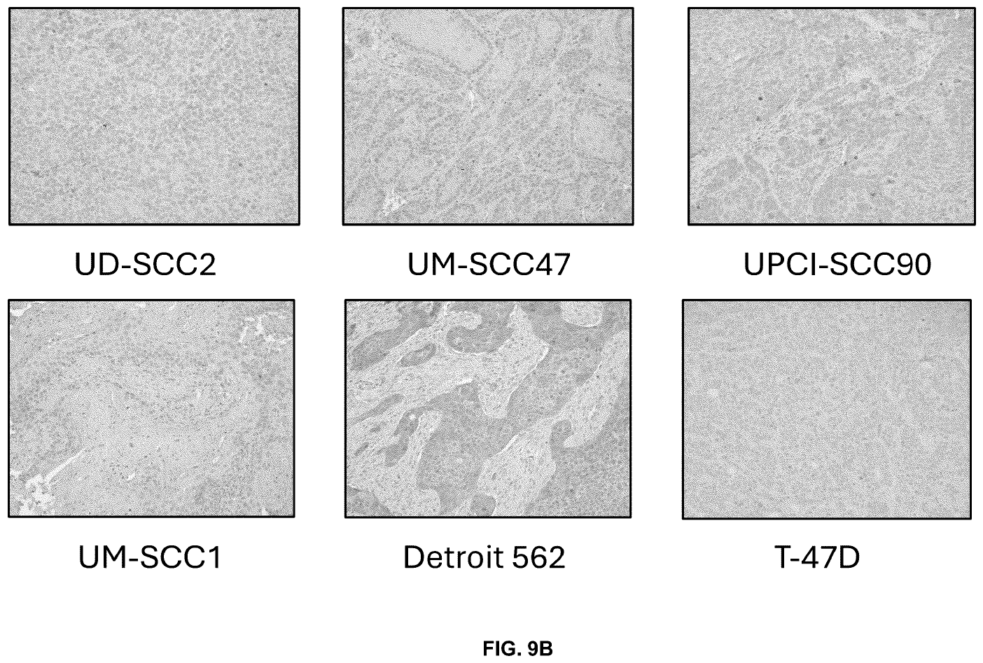

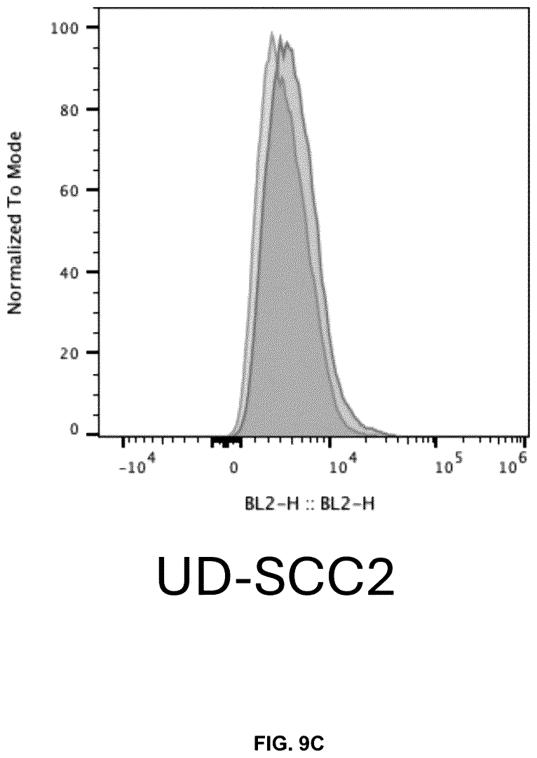

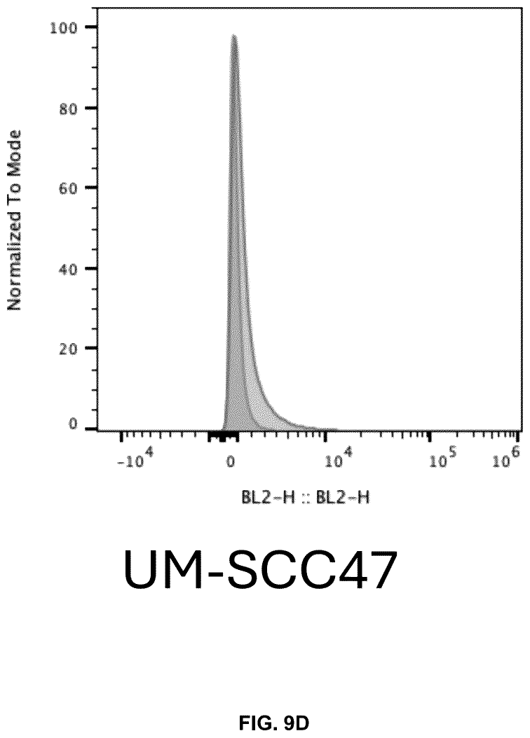

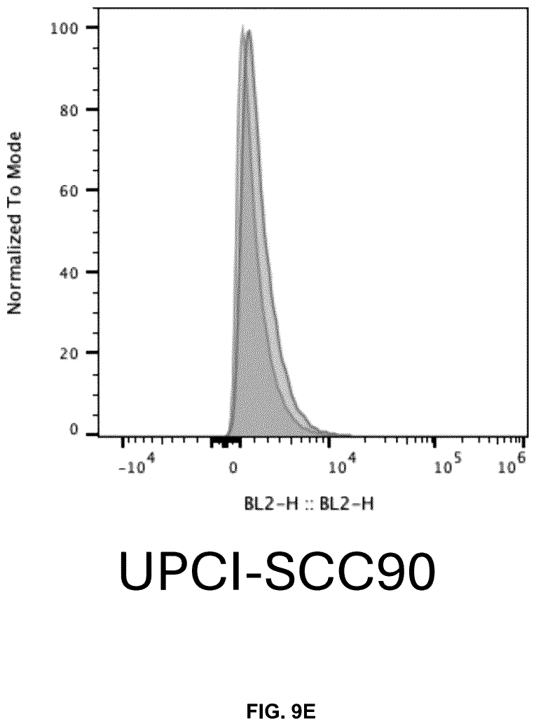

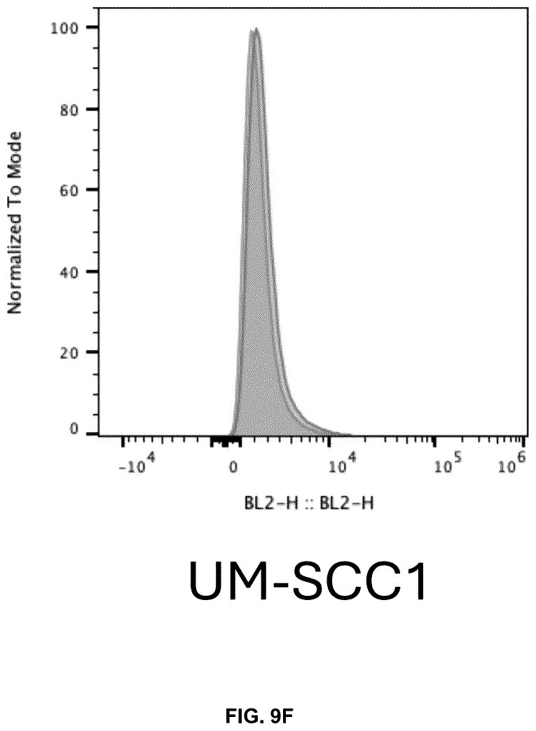

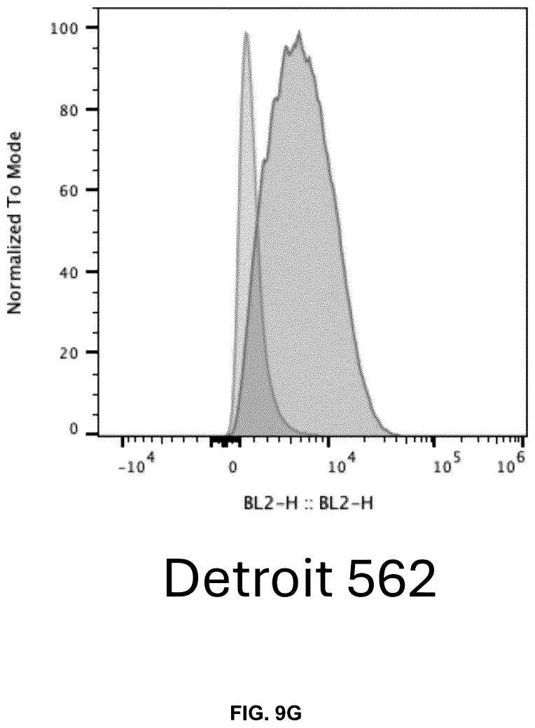

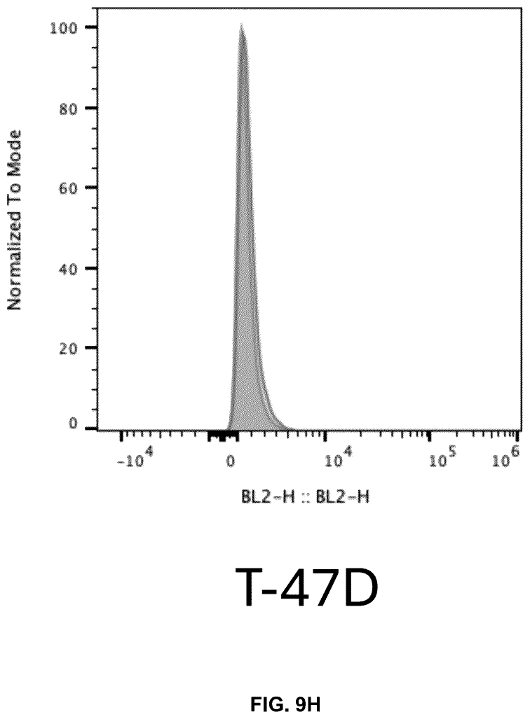

Western Blot was conducted to evaluate MET and phosphorylation of MET (p-MET) expression on a panel of HSNCC cell lines grown under normal growth cell culture. Detroit 562 (HPV-negative) demonstrated high MET and p-MET expression while UM-SCC1 (HPV-negative), UD-SCC2, UM-SCC47, and UPCI SCC90 (all HPV-positive) demonstrated moderate to low MET expression and low p-MET expression. Breast cell line T-47D was identified to have no MET or p-MET expression (FIG. 9A). IHC staining of MET in the cell lines grown as xenografts confirmed our western blot findings, with only Detroit 562 showing significant MET staining (FIG. 9B). Flow cytometry was performed to confirm the specificity of our camelid antibody to MET. As indicated by the right shift, 1E7-Fc showed higher binding to Detroit 562 with minimal to no binding of the other low MET expressing cell lines (FIGS. 9C-9H).

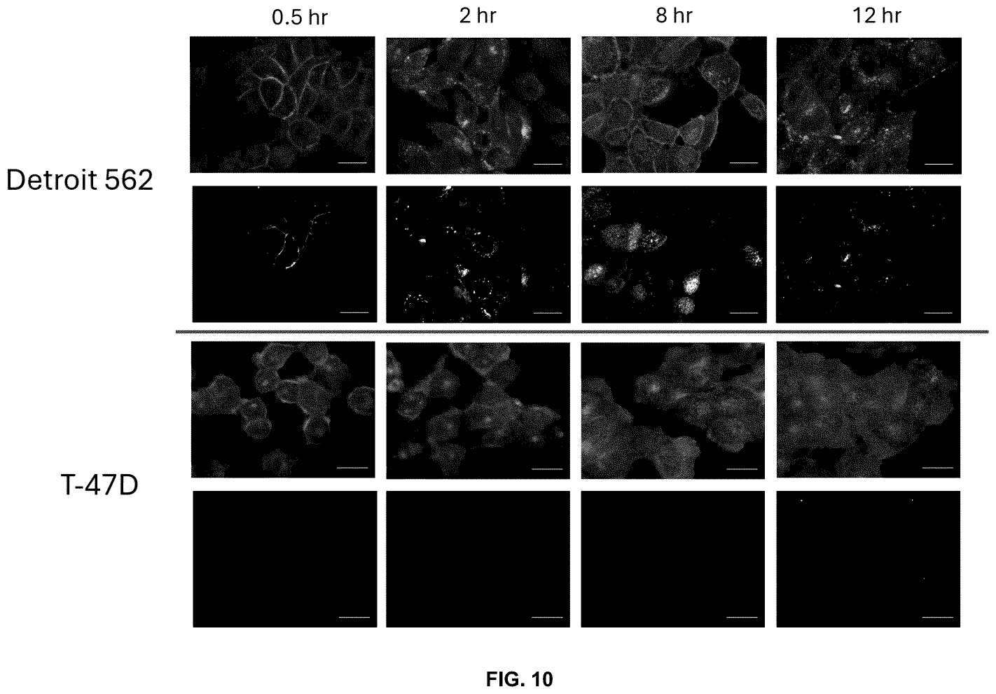

Immunofluorescent live cell imaging was performed on high MET expressing cell line, Detroit 562, to assess the internalization of 1E7-Fc across the plasma membrane. The results demonstrated that binding of camelid antibody occurred within 30 min of exposure and internalization was visualized for up to 8 h after exposure. By 12 h, rapid clearance from cellular compartment was observed. No binding nor internalization of camelid antibody was seen within the negative control cell line, T-47D (FIG. 10).

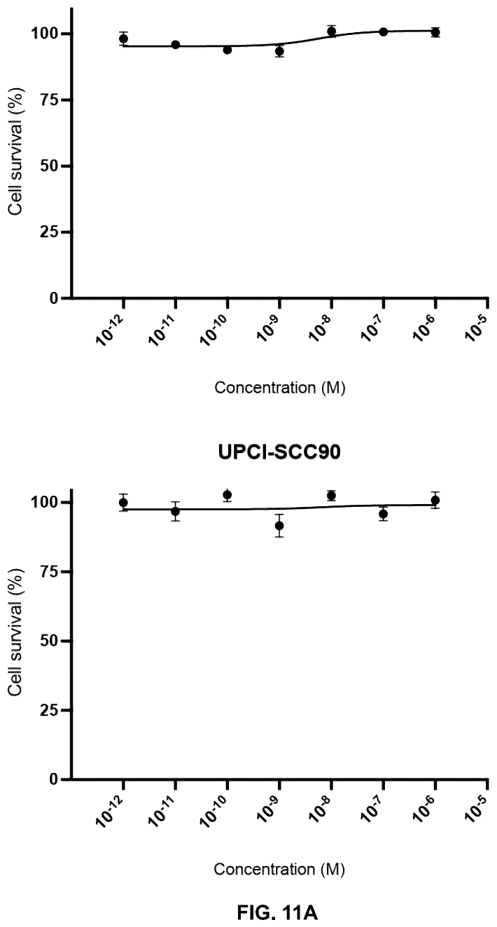

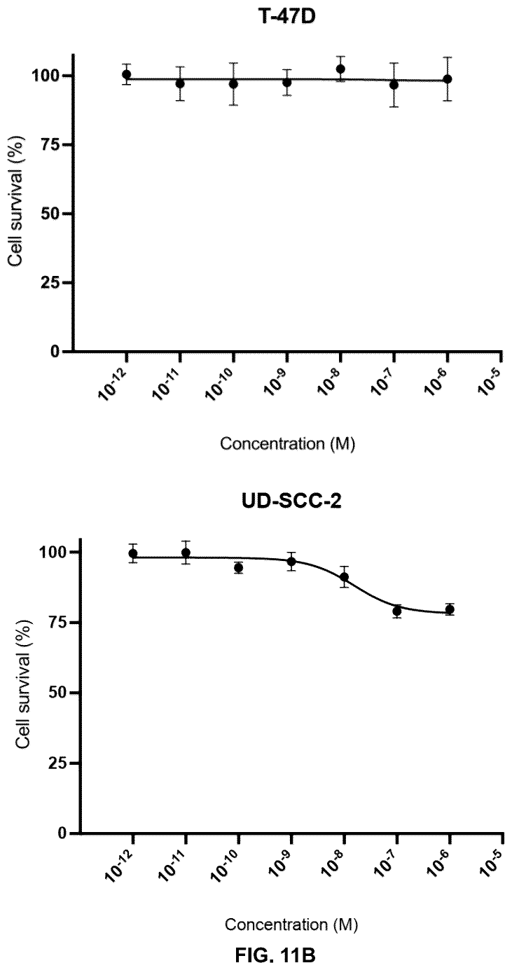

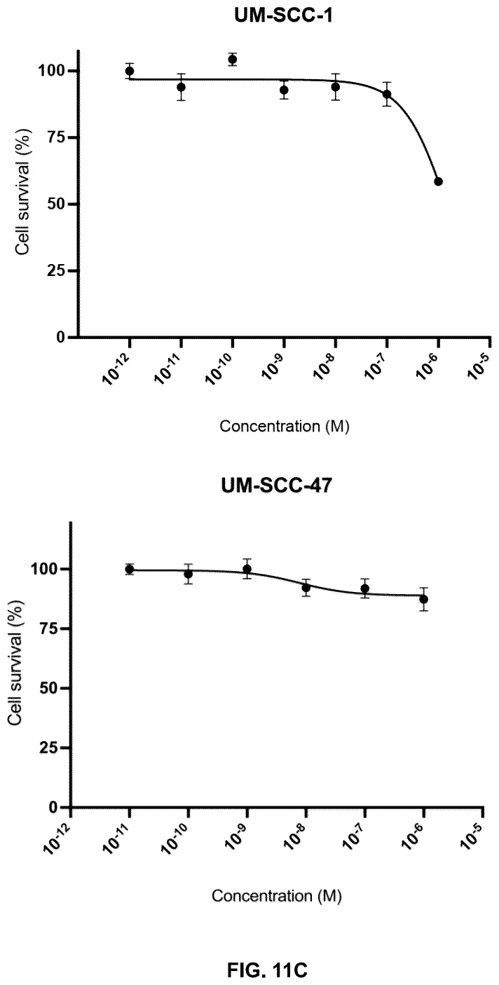

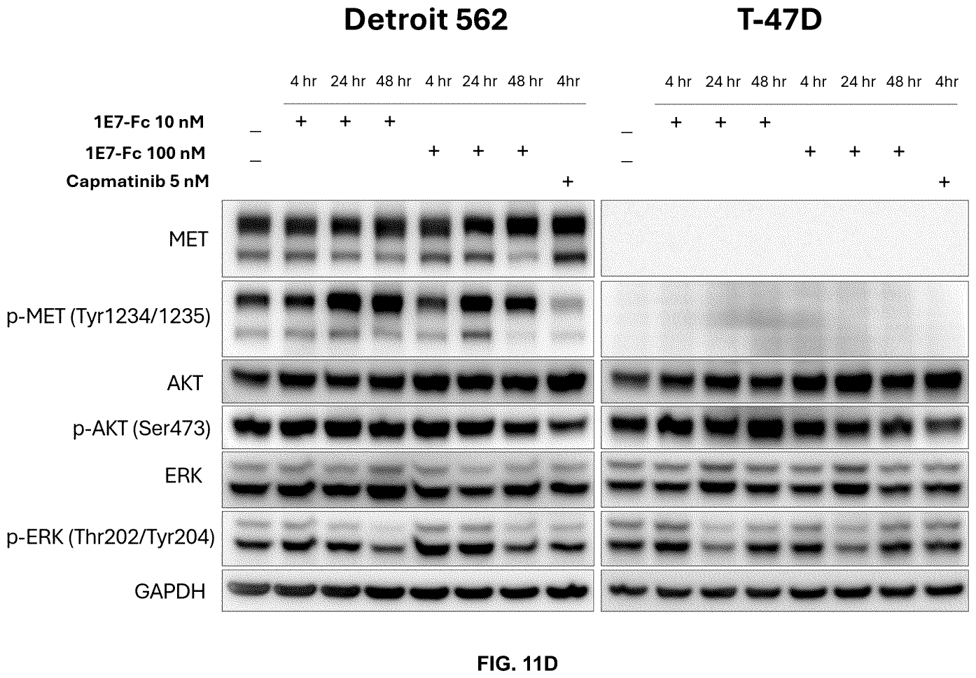

To determine if 1E7-Fc by itself was therapeutic and inhibited MET signaling, both cell proliferation assays and western blotting examining MET downstream signaling were evaluated in Detroit 462 cell and T-47D as a negative control. The MET tyrosine kinase inhibitor capmatinib was used as a positive control for MET downstream signaling. 1E7-Fc exhibited no impact on cell viability even at high concentrations surpassing its binding affinity, indicating it lacks biological activity (FIGS. 11A-11C). This was further supported by no inhibitory effects of 1E7-Fc on MET phosphorylation or downstream signaling of AKT and ERK (FIG. 11D). In contrast, the FDA-approved MET TKI, capmatinib, effectively blocked the phosphorylation of MET as expected (FIG. 11D). Collectively, these findings suggested that 1E7-Fc exerts no biological effect on MET-expressing cells and functions neither as agonist nor antagonist on MET signaling pathway.

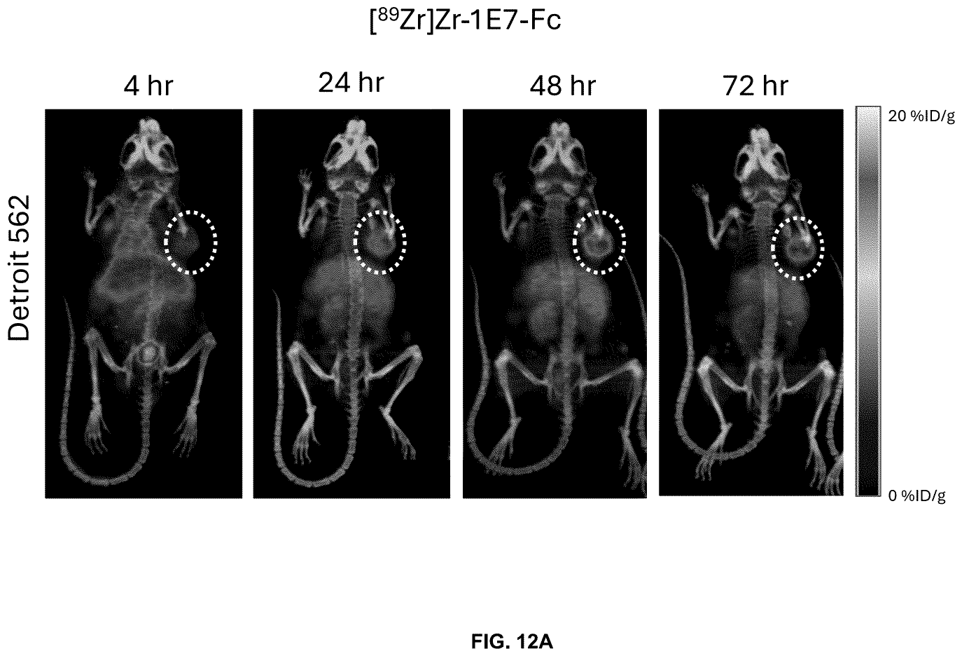

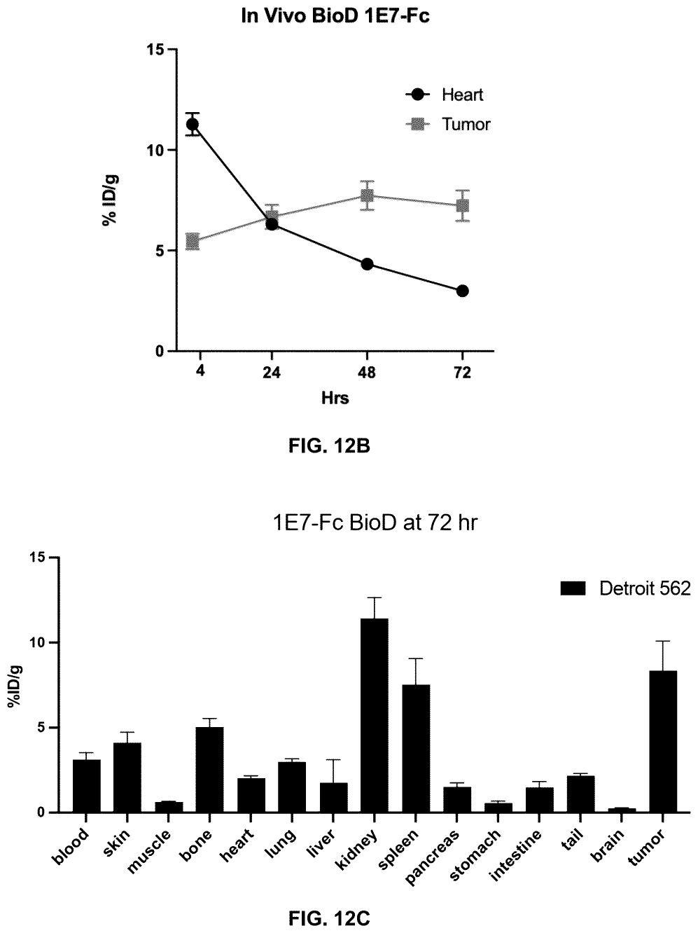

PET/CT imaging was performed on Detroit 562 xenograft models to assess [⁸⁹Zr]Zr-1E7-Fc as an imaging agent to target MET in vivo. [⁸⁹Zr]Zr-1E7-Fc demonstrated rapid localization and high tumor uptake and retention with 8.4% % ID/g 72 hours post injection (FIG. 12A). Additional ROI analysis revealed rapid clearance from the circulatory systems within 24 h post injection while tumor concentration of [⁸⁹Zr]Zr-1E7-Fc increased and remained stable out to 72 h (FIG. 12B). Ex vivo biodistribution was performed at 72 h and the tumor-to-blood radioactivity ratio was calculated to be 2.7. Alongside the tumor, organs associated with small molecular filtration such as kidney and spleen demonstrated high accumulation of [⁸⁹Zr]Zr-1E7-Fc (FIG. 12C).

In this study, we demonstrate that MET is expressed in the majority of HNSCC patient tumors which correlates with a worse prognosis in patients who are HPV-negative. This is consistent with previous reports where overexpression of MET has been observed in 65-80% of HNSCC tumors and has been associated with poor prognosis in patients who are HPV-negative (Bauman et al. 2023, Baschnagel et al. 2014). Given the high MET expression and association with worse outcomes, the development of MET-targeting theranostic agents is warranted.

Previous clinical studies targeting MET with small molecule kinase inhibitors in HNSCC showed mixed results, likely due to inaccurate patient selection based on MET expression levels or lack of a driver role of the MET receptor. MET-directed PET/CT imaging could identify patients with high MET expression who could then be treated with a MET antibody drug conjugate or MET-conjugated radionuclide. In this context, camelid antibodies, with their smaller binding domains, emerge as valuable tools capable of binding inaccessible epitopes with high affinity (Könning et al. 2017, Muyldermans et al. 2021). Previously, we demonstrated 1E7-FC was highly successfully in identifying non-small cell lung cancer lesions with high MET expression attributable to MET amplifications or MET exon 14 skipping mutation (Luo et al. 2024). Building upon this success, this study investigated the efficacy of this specific camelid in detecting HNSCC with elevated MET expression levels.

Similar to what has previously been demonstrated, 1E7-Fc showed high specificity and binding affinity to MET receptors on HNSCC cell lines without causing growth inhibition or cytotoxicity. We also provide evidence that once bound, 1E7-Fc is internalized and taken into the intracellular compartments of MET-expressing HNSCC for an extended period. This not only highlights 1E7-Fc performance as an effective bioimaging agent in PET/CT imaging, but also as a drug or radiotherapy delivery agent. This discovery is crucial as there is currently no PET imaging agent available that successfully targets MET in HNSCC. The current PET imaging gold-standard, 2-deoxy-2 [18F]fluoro-D-glucose ([18F]F-FDG), provides adequate information of primary tumor sights in HNSCC (Almuhaideb et al. 2011). However, [18F]F-FDG uptake is not cancer specific, resulting in substantial noise in regions of high metabolic function and uncertain treatment response assessment (Almuhaideb et al. 2011). Additionally, application of [¹⁸F]F-FDG in HNSCC faces distinct limitations due to the delicate structure of the region and the natural uptake in tissues such as the brain, vocal cords, and cervical muscles (Caldarella et al. 2024). Naturally, these factors potentially affect the scan's prognostic accuracy and highlight the need to introduce additional agents for HNSCC imaging.

MET-targeting PET imaging agents like [⁶⁸Ga]Ga-EMP-100 in renal cell carcinoma (Mittlmeier et al. 2022) and [¹⁸F]F-AH113804 in breast cancer (Arulappu et al. 2016) are under investigation, but neither are antibody-based nor have demonstrated HNSCC application. Clinical trials exploring the therapeutic benefits of MET-targeting monoclonal IgG antibodies have shown disappointing results. For instance, anti-MET antibodies, Onartuzumab (Spigel et al. 2017) and ABT-700 (Strickler et al. 2014), failed to show therapeutic benefits or improve overall survival in late-phase clinical trials. Additionally, the antibody-drug conjugate Telisotuzumab vedotin (ABBV-399) was prematurely terminated due to severe adverse side effects (Camidge et al. 2021, Waqar et al. 2021). There is a need for more innovative approaches in designing theranostic agents to produce better clinical results than those previously achieved. As a variable heavy domain of heavy chain (VHH)-Fc, our camelid-derived antibody (1E7-Fc) offers greater stability, smaller size, and potentially stronger binding to MET than murine-derived monoclonal antibodies undergoing clinical trials. Even when grafted onto a human Fc domain, VHH-Fc constructs maintain a molecular weight of around 80 kDa, resulting in in vivo half-lives compatible with either positron-emitting radionuclide ⁸⁹Zr (t_{1/2}−3.3 d) or the therapeutic radionuclide ⁹⁰Y (t_{1/2}−2.7 d).

### Conclusions

In summary, we demonstrate that the MET receptor is highly expressed in HNSCC and is associated with poor prognosis making it a worthy target for a theranostic. We show that the camelid antibody, 1E7-Fc, has high specificity to the MET receptor and exhibits no cytotoxic effects. Following binding to the MET receptor, internalization of 1E7-Fc was observed, supporting the use of 1E7-Fc as both a PET agent and for the delivery of radionuclide therapy or antibody-drug conjugate therapy. Future studies are warranted to assess the potential of this novel camelid antibody as a theranostic in patients with HNSCC.

### Materials and Methods

### Tissue Microarray (TMA)

Three Oropharyngeal Squamous Cell Carcinoma (OPSCC) TMAs were previously constructed by the University of Wisconsin (UW) Head and Neck Specialized Program of Research Excellence (SPORE) under an IRB-approved protocol. These TMA slides were evaluated for MET expression. Each TMA block consisted of triplicate 1.0-1.5 mm tissue cores of 203 patients treated between 1989 and 2017. Each triplicate consisted of 3 samples of the same tumor tissue to allow maximum data confidence.

### TMA Immunostaining

Multiplex immunostaining was performed on the TMAs using the Opal method following the manufacturer's protocol (Akoya, CA) and the antibodies were applied in sequence as follows: MET/Opal 650 (Cell Signaling Technology, #8198, dilution factor of 1:200, incubation period of 30 min) and pan-cytokeratin (PCK)/Opal 690 (Abcam, ab7735, dilution factor of 1:2000, incubation period of 15 min). Stained tissue microarray slides were then scanned with Vectra 2 (Akoya, CA) at 20X. InForm Tissue Analysis Workflow v.2.4. software (Akoya, CA) was used to create an automated algorithm to segment stroma and epithelium, as well as identify MET expression. Utilizing a spectral library algorithm, PCK was used to detect epithelium, while each individual signal of MET and PCK was unmixed, and applied pseudo colors for image analysis: MET (red), PCK (green), and DAPI (blue). Manual segmentation of tissue compartments was performed: epithelium versus stroma, and subcellular compartments of the nucleus, membrane, and cytoplasm. Samples that were blurry or had less than 100 cells were excluded. In addition, when training the tissue segmentation, each epithelium and stroma algorithm required a minimum of a 92% segmentation accuracy. All cellular antibody expression levels from each image were quantitated as positivity utilizing pixel-based thresholds.

### TCGA MET RNA Expression Analysis

The Cancer Genome Atlas (TCGA) database (https://www.cancer.goc/tcga) was queried for all patients with HNSCC (Cancer Genome Atlas Network 2015). Patient characteristics and MET RNA expression values were extracted from the website database. We compiled a cohort of 486 patients with a diagnosis of HNSCC. Patients with stage IVC metastatic disease or those that did not have progression-free survival (PFS) data were excluded from the analysis. Fragments Per Kilobase per Million mapped fragments (FPKM) values were utilized to categorize patients into High and Low MET expression groups, with the mean FPKM value serving as the threshold. Patients were further subcategorized based on HPV positivity from p16 testing and patients without HPV status were assumed to be negative.

### Camelid Antibody

The 1E7 was obtained from screening a camelid antibody phage display library as previously described (Example 1 and Luo et al. 2024). The sequence was cloned into a TGEX human Fc expression vector, and plasmid DNA was purified using PureLink HiPure plasmid maxiprep kits. Purified plasmids were transfected into ExpiCHO-S cells using an Expifectamine CHO Transfection Kit with 1 μg of plasmid DNA and 3.2 μL of Expifectamine CHO Reagent per milliliter of suspension culture. Cell culture technique and kit application was executed as previously described (Luo et al. 2024). The 1E7-Fc antibody was captured using protein A and purified by size exclusion chromatography with an ÄKTApure system with a Superdex 200 PG column. Samples were fractionated with phosphate-buffered saline (PBS) at 0.5 mL/min, and chromatograms were obtained by monitoring UV absorbance at 280 nM. Eluted protein fractions corresponding to the theoretical molecular mass of 1E7-Fc were collected, pooled, diluted to 1 mg/mL in PBS, dispensed into aliquots, and flash frozen.

### Cell Lines and Culture

HNSCC cell lines (HPV-positive: UD-SCC2, UM-SCC47, UPCI: SCC90; HPV-negative: UM-SCC1, Detroit 562) were obtained from the UW Head and Neck SPORE cell line repository and maintained in Dulbecco's Modified Eagle Medium (DMEM), supplemented with 10% FBS and 1% penicillin and streptomycin at 37° C. and 5% CO₂. T-47D (Met-negative breast carcinoma) was cultured in RPMI with 10% FBS, 1%, penicillin and streptomycin, and 1% L-Glutamine. The identity of each cell lines was authenticated using short-tandem repeat profiling and routine mycoplasma testing was performed with MycoStrip (Invivogen).

### Immunoblot Analysis

Cells were seeded in 10-cm dishes and treated with either control (i.e., PBS), 10 nM, or 100 nM 1E7-Fc when cells reached about 75% confluency, and then cells were harvested 4, 24, or 48 h after treatment. Capmatinib (5 nM) was used as a positive control for inhibition of MET signaling. Cell lysates were prepared as previously described (Könning et al. 2017). Equal amounts of protein were analyzed by sodium dodecyl-sulfate polyacrylamide gel electrophoresis (SDS-PAGE), transferred to polyvinylidene fluoride membranes, and probed with specific primary antibodies. Targets were detected with horseradish peroxidase (HRP)-conjugated secondary antibodies (BioRad) using Enhanced Chemiluminescence western blot substrates (Pierce) and imaged on a LI-COR Odyssey Fc. The specific antibodies are as listed: MET, phospho-MET (Tyr1234/1235), AKT, phosphor-AKT (Ser473), p44/42 MAPK (Erk1/2), phosphor-p44/42 MAPK (Erk1/2) *Thr202/Try204), GAPDH.

### Xenograft IHC

Each xenograft tumor (UD-SCC2, UM-SCC47, UPCI: SCC90, UM-SCC1, Detroit 562, and T-47D) was fixed in 10% neutral-buffered formalin and embedded in paraffin blocks. Five-μm sections of formalin-fixed, paraffin-embedded samples were deparaffinized with Xylene and hydrated through graded solutions of ethanol. Antigen retrieval was conducted in sodium citrate retrieval buffer (pH 6.0) followed by washing in running water. The slides were washed in PBS and then incubated with a 0.3% hydrogen peroxide solution. Blocking was carried out using 10% goat serum in PBS and then incubated with the MET antibody (Cell Signaling Technology, #8198) diluted in 1% goat serum in PBS containing 0.1% Triton X-100 overnight at 4° C. The slides were washed with PBS the next day; secondary antibody (Signal Stain Boost IHC Detection Reagent (HRP, Rabbit), CST #8114) was used. Staining was detected using diaminobenzidine (Vector Laboratories, Inc. #SK-4100). The slides were counterstained with 1:10 hematoxylin (Thermo Scientific, #TA-125-MH) solution for 2 min, then dehydrated in ethanol and xylene solutions and sections were covered with coverslip with Cytoseal (Thermo Scientific #8312-4). Images were obtained on an Olympus BX51 microscope (Olympus America, Inc).

### Flow Cytometry

Single cell suspension of each cell line was prepared by trypsinization. About 0.5×10⁶ cells were incubated with 100 nM of 1E7-Fc for 30 min on ice. Then cells were washed and stained with 1 μg/mL Goat anti-Human IgG Fc, PE (eBioscience) for 30 min on ice in the dark. Cells were washed three times and resuspended in flow cytometry staining buffer (2% FBS and 0.1% NaN₃ in PBS). Data was collected with the Attune Flow Cytometer (Thermo Fisher), and at least 10,000 viable cells were gated and analyzed with FlowJo.

### Immunofluorescent Live Cell Imaging

Cells were seeded into an eight-well chamber coverslip slide (#1.5 thickness) at approximately 40,000 to 80,000 cells per well depending on cell type. After incubating at 37° C. for 48 h, cells were stained with a green CellMask Plasma Membrane stain (C37608, Thermo Fisher Scientific) and blue Hoechst 33342 (62249, Thermo Fisher Scientific) in accordance with manufacturer procedure. The stained cells were then placed in a temperature- and CO₂-controlled Tokei Hit Biochamber and imaged using a Nikon Ti2 Spinning Disk Confocal Microscope at an objective of 40X with excitation laser 405, 488, and 640 nM. At the beginning of the imaging sequence, cells were treated with 1 μM Alexa Fluor 647-labeled 1E7-Fc antibody (A20186, Thermo Fisher Scientific) for 15 min. The cell medium was then suctioned out and phenol red-free DMEM media was added (21-064-029, Thermo Fisher Scientific). Images were acquired immediately prior to treatment and every 5 min for the first 3 h post-treatment, and then every 30 min for the next 9 h. Images were processed using Nikon NIS-Elements imaging software and ImageJ.

### Proliferation Assay

Cells were plated in 96-well plates at densities ranging from 3,000 to 6,000 cells per well according to each cell growth rate. After overnight incubation, cells were treated with indicated doses of 1E7-Fc or control (PBS) and incubated for 72 h. After 72 h of drug treatment, Cell Counting Kit-8 reagent was added (Dojindo Molecular Technologies) and absorbance measured at 450 nM on a SpectraMax i3 plate reader (Molecular Devices). The absorbance of treated wells was normalized to control wells, and the half-maximal inhibitory concentration (IC₅₀) values were calculated using GraphPad Prism.

### Animal Models

All animal studies were conducted under a protocol approved by the University of Wisconsin Institutional Animal Care and Use Committee. Four-to-six-week-old female Hsd: athymic Nude-Foxn1nu mice (Envigo) were used for biodistribution and nuclear imaging studies. Detroit 562 xenografts were established by subcutaneous injection of cells (1×10⁶ cells in 100 μl) mixed with Matrigel (1:1, Corning) above the right shoulder blade. Tumors were allowed to grow for a month for the nuclear imaging experiment.

### Bioconjugation and Radiochemistry

1E7-Fc was conjugated to p-SCN-Bn-Deferoxamine (Macrocyclics). Zirconium-89 [⁸⁹Zr] was purchased from the UW Medical Physics Department (Madison, WI). Radiolabeling of 1E7-Fc with ⁸⁹Zr was accomplished using traditional methods previously described and purified with PD-10 columns (GE Healthcare).

### PET/CT Imaging

Images were acquired on Inveon microPET/computed tomography (CT) Scanner (Siemens Medical Solutions) and performed by the Small Animal Irradiation Facility (SAIRF) at the UW Carbone Cancer Center. Mice (n=4) were injected with 9.25 MBq of [⁸⁹Zr]Zr-1E7 Fc by tail vein injection. Mice were anesthetized by inhaling 2.5% isoflurane. Images were recorded at 4, 24, 48, and 72 h time points. PET list mode data were acquired for 80 million counts using a gamma ray energy window of 350-650 KeV and coincidence timing window of 3.428 ns. A CT-based attenuation correction was performed for approximately 10 min with 80 kVp, 1 mA, 220 rotation degrees in 120 rotation steps, 250 ms exposure time, and subsequently reconstructed using a Shepp-Logan filter with 210-μ isotropic voxels. Scans were reconstructed using 3-dimensional ordered-subset expectation maximization (2 iterations, 16 subsets) with a maximum a posteriori probability algorithm (OSEM3DMAP). Two-dimension (2D) images and maximum intensity projections (MIPs) were analyzed, and region of interest (ROI) were manually drawn using Inveon Research Workspace (Siemens medical Solutions). ROI analysis of the images was used to determine tracer uptake in major organs or tissues and quantitative results are given as percentage injected dose per gram of tissue (% ID/g).

### Biodistribution

Biodistribution was performed after the final PET/CT scans. Major organs, tissues, and blood were collected, and the radioactivity in each sample was measured using a WIZARD2 automatic gamma counter (PerkinElmer). Count data were background and decay-corrected, and % ID/g for each tissue sample was calculated by normalization to the total amount of activity injected into each mouse.

### Statistical Analyses

In vitro experiments were repeated three times and statistical analyses were carried out using a Student's t-test or one-way ANOVA. Data are presented as the mean±standard error of the mean (SEM). PFS was calculated with Kaplan-Meier curves and PFS differences were determined with Gehan-Breslow-Wilcoxon test. A p value of <0.05 was considered statistically significant. All graphs and in vitro analyses were performed and graphed using GraphPad Prism version 10.0.3 (Prism) or RStudio (2021.09.0).

### Abbreviations

% ID/g, percentage injected dose per gram; 2D, Two-dimension; [¹⁸F]F-FDG, 2-deoxy-2 [18F]fluoro-D-glucose), DMEM, Dulbecco's Modified Eagle Medium; EGFR, epidermal growth factor receptor; FPKM, Fragments Per Kilobase per Million mapped fragments; HGFR, hepatocyte growth factor receptor; HNSCC, head and neck squamous cell carcinoma; HPV, human papillomavirus; IgG1, immunoglobulin G1; HRP, horseradish peroxidase; IC₅₀, half-maximal inhibitory concentration; IgG Fc, IgG Fc chain; IHC, immunohistochemistry; JAK, Janus kinase/signal transducer and transcription activator; MAPK, mitogen-activated protein kinase; MET, Mesenchymal Epithelial Transition; MIP, maximum intensity projection; NF-κB, nuclear factor kappa B; PBS, phosphate-buffered saline; PCK, pan-cytokeratin; PET/CT, positron emission tomography/computed tomography; PFS, progression-free survival; PI3K, phosphatidylinositol-3 kinase; pMET, phosphorylation of MET; ROI, region of interest; SDS-PAGE, sodium dodecyl-sulfate polyacrylamide gel electrophoresis; SPORE, Specialized Program of Research Excellence; TCGA, Cancer Genome Atlas; TKI, tyrosine kinase inhibitor; TMA, tissue microarray; VHH, variable heavy domain of heavy chain; UW, University of Wisconsin.

### REFERENCES

Aebersold D M, Kollar A, Beer K T, Laissue J, Greiner R H, Djonov V. Involvement of the hepatocyte growth factor/scatter factor receptor c-met and of Bcl-xL in the resistance of oropharyngeal cancer to ionizing radiation. Int J Cancer. 2001 Feb. 20; 96 (1): 41-54.
Akervall J, Guo X, Qian C N, Schoumans J, Leeser B, Kort E, Cole A, Resau J, Bradford C, Carey T, Wennerberg J, Anderson H, Tennvall J, Teh B T. Genetic and expression profiles of squamous cell carcinoma of the head and neck correlate with cisplatin sensitivity and resistance in cell lines and patients. Clin Cancer Res. 2004 Dec. 15; 10 (24): 8204-13.
Almuhaideb A, Papathanasiou N, Bomanji J. 18F-FDG PET/C T imaging in oncology. Ann Saudi Med. 2011 January-February; 31 (1): 3-13.
Arulappu A, Battle M, Eisenblaetter M, McRobbie G, Khan I, Monypenny J, Weitsman G, Galazi M, Hoppmann S, Gazinska P, Wulaningsih W, Dalsgaard G T, Macholl S, Ng T. c-Met PET Imaging Detects Early-Stage Locoregional Recurrence of Basal-Like Breast Cancer. J Nucl Med. 2016 May; 57 (5): 765-70. doi: 10.2967/jnumed.115.164384. Epub 2015 Dec. 3. PMID: 26635342.Arulappu A, Battle M, Eisenblaetter M, et al. c-Met PET Imaging Detects Early-Stage Locoregional
Recurrence of Basal-Like Breast Cancer. J Nucl Med. 2016; 57 (5): 765-770. Asaadi Y, Jouneghani F F, Janani S, Rahbarizadeh F. A comprehensive comparison between camelid nanobodies and single chain variable fragments. Biomark Res. 2021 Dec. 4; 9 (1): 87. doi: 10.1186/s40364-021-00332-6.
Baschnagel A M, Galoforo S, Thibodeau B J, Ahmed S, Nirmal S, Akervall J, Wilson G D. Crizotinib Fails to Enhance the Effect of Radiation in Head and Neck Squamous Cell Carcinoma Xenografts. Anticancer Res. 2015 November; 35 (11): 5973-82.
Baschnagel A M, Tonlaar N, Eskandari M, Kumar T, Williams L, Hanna A, Pruetz B L, Wilson G D. Combined CD44, c-MET, and EGFR expression in p16-positive and p16-negative head and neck squamous cell carcinomas. J Oral Pathol Med. 2017 March; 46 (3): 208-213.
Baschnagel A M, Williams L, Hanna A, Chen P Y, Krauss D J, Pruetz B L, Akervall J, Wilson G D. c-Met expression is a marker of poor prognosis in patients with locally advanced head and neck squamous cell carcinoma treated with chemoradiation. Int J Radiat Oncol Biol Phys. 2014 Mar. 1; 88 (3): 701-7.
Baschnagel A M, Kaushik S, Durmaz A, Goldstein S, Ong I M, Abel L, Clark P A, Gurel Z, Leal T, Buehler D, Iyer G, Scott J G, Kimple R J. Development and characterization of patient-derived xenografts from non-small cell lung cancer brain metastases. Sci Rep. 2021 Jan. 28; 11 (1): 2520.
Bauman J E, Ohr J, Gooding W E, Ferris R L, Duvvuri U, Kim S, Johnson J T, Soloff A C, Wallweber G, Winslow J, Gaither-Davis A, Grandis J R, Stabile L P. Phase I Study of Ficlatuzumab and Cetuximab in Cetuximab-Resistant, Recurrent/Metastatic Head and Neck Cancer. Cancers (Basel). 2020 Jun. 11; 12 (6): 1537.
Bauman J E, Saba N F, Roe D, Bauman J R, Kaczmar J, Bhatia A, Muzaffar J, Julian R, Wang S, Bearelly S, Baker A, Steuer C, Giri A, Burtness B, Centuori S, Caulin C, Klein R, Saboda K, Obara S, Chung C_{H}. Randomized Phase II Trial of Ficlatuzumab With or Without Cetuximab in Pan-Refractory, Recurrent/Metastatic Head and Neck Cancer. J Clin Oncol. 2023 Aug. 1; 41 (22): 3851-3862.
Ben Abderrazek R, Vincke C, Hmila I, Saerens D, Abidi N, El Ayeb M, Muyldermans S, Bouhaouala-Zahar B. Development of Cys38 knock-out and humanized version of NbAahII10 nanobody with improved neutralization of AahII scorpion toxin. Protein Eng Des Sel. 2011 September; 24 (9): 727-35.
Birchmeier C, Birchmeier W, Gherardi E, Vande Woude G F. Met, metastasis, motility and more. Nat Rev Mol Cell Biol. 2003 December; 4 (12): 915-25.
Birchmeier C, Gherardi E. Developmental roles of HGF/S F and its receptor, the c-Met tyrosine kinase. Trends Cell Biol. 1998 October; 8 (10): 404-10.
Caldarella C, De Risi M, Massaccesi M, Miccichè F, Bussu F, Galli J, Rufini V, Leccisotti L. Role of ¹⁸F-FDG PET/C T in Head and Neck Squamous Cell Carcinoma: Current Evidence and Innovative Applications. Cancers (Basel). 2024 May 16; 16 (10): 1905.
Camidge D R, Morgensztern D, Heist R S, Barve M, Vokes E, Goldman J W, Hong D S, Bauer T M, Strickler J H, Angevin E, Motwani M, Parikh A, Sun Z, Bach B A, Wu J, Komarnitsky P B, Kelly K. Phase I Study of 2- or 3-Week Dosing of Telisotuzumab Vedotin, an Antibody-Drug Conjugate Targeting c-Met, Monotherapy in Patients with Advanced Non-Small Cell Lung Carcinoma. Clin Cancer Res. 2021 Nov. 1; 27 (21): 5781-5792.
Camidge D R, Moran T, Demedts I, Grosch H, Mileham K, Molina J, Juan-Vidal O, Bepler G, Goldman J W, Park K, Wallin J, Wijayawardana S R, Wang X A, Wacheck V, Smit E. A Randomized, Open-Label Phase II Study Evaluating Emibetuzumab Plus Erlotinib and Emibetuzumab Monotherapy in MET Immunohistochemistry Positive NSCLC Patients with Acquired Resistance to Erlotinib. Clin Lung Cancer. 2022 June; 23 (4): 300-310.
Cancer Genome Atlas Network. Comprehensive genomic characterization of head and neck squamous cell carcinomas. Nature. 2015 Jan. 29; 517 (7536): 576-82.
Cancer Genome Atlas Research Network. Comprehensive molecular profiling of lung adenocarcinoma. Nature. 2014 Jul. 31; 511 (7511): 543-50. doi: 10.1038/nature13385. Epub 2014 Jul. 9. Erratum in: Nature. 2014 Oct. 9; 514 (7521): 262.
De Groeve K, Deschacht N, De Koninck C, Caveliers V, Lahoutte T, Devoogdt N, Muyldermans S, De Baetselier P, Raes G. Nanobodies as tools for in vivo imaging of specific immune cell types. J Nucl Med. 2010 May; 51 (5): 782-9.
De Mello, R. A. et al. The Role of MET Inhibitor Therapies in the Treatment of Advanced Non-Small Cell Lung Cancer. Journal of Clinical Medicine 9, 1918 (2020).
Desai A, Cuellar S. The Current Landscape for METex14 Skipping Mutations in Non-Small Cell Lung Cancer. J Adv Pract Oncol. 2022 July; 13 (5): 539-544.
Digumarthy S R, Mendoza D P, Zhang E W, Lennerz J K, Heist R S. Clinicopathologic and Imaging Features of Non-Small-Cell Lung Cancer with MET Exon 14 Skipping Mutations. Cancers (Basel). 2019 Dec. 17; 11 (12): 2033.
Jagoda E M, Lang L, Bhadrasetty V, Histed S, Williams M, Kramer-Marek G, Mena E, Rosenblum L, Marik J, Tinianow J N, Merchant M, Szajek L, Paik C, Cecchi F, Raffensperger K, Jose-Dizon J M, Bottaro D P, Choyke P. Immuno-PET of the hepatocyte growth factor receptor Met using the 1-armed antibody onartuzumab. J Nucl Med. 2012 October; 53 (10): 1592-600.
Fan S, Gao M, Meng Q, Laterra J J, Symons M H, Coniglio S, Pestell R G, Goldberg I D, Rosen E M. Role of N F-kappaB signaling in hepatocyte growth factor/scatter factor-mediated cell protection. Oncogene. 2005 Mar. 3; 24 (10): 1749-66.
Gherardi E, Birchmeier W, Birchmeier C, Vande Woude G. Targeting MET in cancer: rationale and progress. Nat Rev Cancer. 2012 Jan. 24; 12 (2): 89-103.
Glumac P M, Gallant J P, Shapovalova M, Li Y, Murugan P, Gupta S, Coleman I M, Nelson P S, Dehm S M, LeBeau A M. Exploitation of CD133 for the Targeted Imaging of Lethal Prostate Cancer. Clin Cancer Res. 2020 Mar. 1; 26 (5): 1054-1064.
Han Z, Zhang T, Ke M, Qiao L, Wu Z, Guan Z, Li Z, Wu Z, Sun X. PET imaging of c-Met activity in NSCLC using 18F Labelled c-MET inhibitor. Journal of Nuclear Medicine May 2021, 62 (supplement 1) 148.
Heukers R, Mashayekhi V, Ramirez-Escudero M, de Haard H, Verrips T C, van Bergen En Henegouwen PMP, Oliveira S. VHH-Photosensitizer Conjugates for Targeted Photodynamic Therapy of Met-Overexpressing Tumor Cells. Antibodies (Basel). 2019 Apr. 4; 8 (2): 26.
Hong L, Zhang J, Heymach J V, Le X. Current and future treatment options for MET exon 14 skipping alterations in non-small cell lung cancer. Ther Adv Med Oncol. 2021 Feb. 15; 13:1758835921992976.
Johnson G L, Lapadat R. Mitogen-activated protein kinase pathways mediated by ERK, JNK, and p38 protein kinases. Science. 2002 Dec. 6; 298 (5600): 1911-2.
Kim C H, Koh Y W, Han J H, Kim J W, Lee J S, Baek S J, Hwang H S, Choi E C. c-Met expression as an indicator of survival outcome in patients with oral tongue carcinoma. Head Neck. 2010 December; 32 (12): 1655-64.
Kim J H, Kim B J, Kim H S. Clinicopathological impacts of high c-Met expression in head and neck squamous cell carcinoma: a meta-analysis and review. Oncotarget. 2017 Sep. 28; 8 (68): 113120-113128.
Kim Y J, Go H, Wu H G, Jeon Y K, Park S W, Lee S H. Immunohistochemical study identifying prognostic biomolecular markers in nasopharyngeal carcinoma treated by radiotherapy. Head Neck. 2011 October; 33 (10): 1458-66.
Knowles L M, Stabile L P, Egloff A M, Rothstein M E, Thomas S M, Gubish C T, Lerner E C, Seethala R R, Suzuki S, Quesnelle K M, Morgan S, Ferris R L, Grandis J R, Siegfried J M. HGF and c-Met participate in paracrine tumorigenic pathways in head and neck squamous cell cancer. Clin Cancer Res. 2009 Jun. 1; 15 (11): 3740-50.
Kochanny S E, Worden F P, Adkins D R, Lim D W, Bauman J E, Wagner S A, Brisson R J, Karrison T G, Stadler W M, Vokes E E, Seiwert T Y. A randomized phase 2 network trial of tivantinib plus cetuximab versus cetuximab in patients with recurrent/metastatic head and neck squamous cell carcinoma. Cancer. 2020 May 15; 126 (10): 2146-2152.
Könning D, Zielonka S, Grzeschik J, Empting M, Valldorf B, Krah S, Schröter C, Sellmann C, Hock B, Kolmar H. Camelid and shark single domain antibodies: structural features and therapeutic potential. Curr Opin Struct Biol. 2017 August; 45:10-16.
Li L, Sun Z, Huang X, Li X, Sun L, Zhang L, Zhang X, Ye L, Yuan J, Mao L, Li G. Role of c-Met expression on prognosis of head and neck cancer: A literature review and meta-analysis. Head Neck. 2019 June; 41 (6): 1999-2006.
Li K, Tavaré R, Zettlitz K A, Mumenthaler S M, Mallick P, Zhou Y, Marks J D, Wu A M. Anti-MET immunoPET for non-small cell lung cancer using novel fully human antibody fragments. Mol Cancer Ther. 2014 November; 13 (11): 2607-17.
Lim Y C, Han J H, Kang H J, Kim Y S, Lee B H, Choi E C, Kim C_{H}. Overexpression of c-Met promotes invasion and metastasis of small oral tongue carcinoma. Oral Oncol. 2012 November; 48 (11): 1114-9.
Liu L, Shi H, Liu Y, Anderson A, Peterson J, Greger J, Martin A M, Gilmer T M. Synergistic effects of foretinib with HER-targeted agents in MET and HER 1- or HER2-coactivated tumor cells. Mol Cancer Ther. 2011 March; 10 (3): 518-30.
Luo N Y, Minne R L, Gallant J P, Gunaratne G S, West J L, Javeri S, Robertson A J, Lake E W, Engle J W, Mixdorf J C, Aluicio-Sarduy E, Nickel K P, Hernandez R, Kimple R J, Baschnagel A M, LeBeau A M. Development of an Engineered Single-Domain Antibody for Targeting MET in Non-Small Cell Lung Cancer. Bioconjug Chem. 2024 Mar. 20; 35 (3): 389-399.
Lutterbach B, Zeng Q, Davis L J, Hatch H, Hang G, Kohl N E, Gibbs J B, Pan B S. Lung cancer cell lines harboring MET gene amplification are dependent on Met for growth and survival. Cancer Res. 2007 Mar. 1; 67 (5): 2081-8.
Lüttich L, Besso M J, Heiden S, Koi L, Baumann M, Krause M, Dubrovska A, Linge A, Kurth I, Peitzsch C. Tyrosine Kinase c-MET as Therapeutic Target for Radiosensitization of Head and Neck Squamous Cell Carcinomas. Cancers (Basel). 2021 Apr. 14; 13 (8): 1865.
Martinelli I, Modica C, Chiriaco C, Basilico C, Hughes J M, Corso S, Giordano S, Comoglio P M, Vigna E. hOA-DN30: a highly effective humanized single-arm MET antibody inducing remission of ‘MET-addicted’ cancers. J Exp Clin Cancer Res. 2022 March 29; 41 (1): 112. doi: 10.1186/s13046-022-02320-6. Erratum in: J Exp Clin Cancer Res. 2022 May 24; 41 (1): 180.
Mittlmeier L M, Todica A, Gildehaus F J, Unterrainer M, Beyer L, Brendel M, Albert N L, Ledderose S T, Vettermann F J, Schott M, Rodler S, Marcon J, Ilhan H, Cyran C C, Stief C G, Staehler M, Bartenstein P. 68Ga-EMP-100 PET/C T-a novel ligand for visualizing c-MET expression in metastatic renal cell carcinoma-first in-human biodistribution and imaging results. Eur J Nucl Med Mol Imaging. 2022 April; 49 (5): 1711-1720.
Muyldermans S. Applications of Nanobodies. Annu Rev Anim Biosci. 2021 Feb. 16; 9:401-421.
Muyldermans S. Nanobodies: natural single-domain antibodies. Annu Rev Biochem. 2013; 82:775-97.
Nisa L, Francica P, Giger R, Medo M, Elicin O, Friese-Hamim M, Wilm C, Stroh C, Bojaxhiu B, Quintin A, Caversaccio M D, Dettmer M S, Buchwalder M, Brodie T M, Aebersold D M, Zimmer Y, Carey T E, Medová M. Targeting the MET Receptor Tyrosine Kinase as a Strategy for Radiosensitization in Locoregionally Advanced Head and Neck Squamous Cell Carcinoma. Mol Cancer Ther. 2020 February; 19 (2): 614-626.
Okuda K, Sasaki H, Yukiue H, Yano M, Fujii Y. Met gene copy number predicts the prognosis for completely resected non-small cell lung cancer. Cancer Sci. 2008 November; 99 (11): 2280-5.
Oliveira S, Schiffelers R M, van der Veeken J, van der Meel R, Vongpromek R, van Bergen En Henegouwen P M, Storm G, Roovers R C. Downregulation of EGFR by a novel multivalent nanobody-liposome platform. J Control Release. 2010 Jul. 14; 145 (2): 165-75.
Perk L R, Stigter-van Walsum M, Visser G W, Kloet R W, Vosjan M J, Leemans C R, Giaccone G, Albano R, Comoglio P M, van Dongen G A. Quantitative PET imaging of Met-expressing human cancer xenografts with 89Zr-labelled monoclonal antibody DN30. Eur J Nucl Med Mol Imaging. 2008 October; 35 (10): 1857-67.
Peschard P, Fournier T M, Lamorte L, Naujokas M A, Band H, Langdon W Y, Park M. Mutation of the c-Cb1 TKB domain binding site on the Met receptor tyrosine kinase converts it into a transforming protein. Mol Cell. 2001 November; 8 (5): 995-1004.
Ponzetto C, Bardelli A, Zhen Z, Maina F, dalla Zonca P, Giordano S, Graziani A, Panayotou G, Comoglio P M. A multifunctional docking site mediates signaling and transformation by the hepatocyte growth factor/scatter factor receptor family. Cell. 1994 Apr. 22; 77 (2): 261-71.
Pool M, Terwisscha van Scheltinga AGT, Kol A, Giesen D, de Vries EGE, Lub-de Hooge M N. ⁸⁹Zr-Onartuzumab PET imaging of c-MET receptor dynamics. Eur J Nucl Med Mol Imaging. 2017 August; 44 (8): 1328-1336.
Reungwetwattana T, Liang Y, Zhu V, Ou S I. The race to target MET exon 14 skipping alterations in non-small cell lung cancer: The Why, the How, the Who, the Unknown, and the Inevitable. Lung Cancer. 2017 January; 103:27-37.
Riechmann L, Muyldermans S. Single domain antibodies: comparison of camel V H and camelised human V H domains. J Immunol Methods. 1999 Dec. 10; 231 (1-2): 25-38.
Rolle C E, Tan Y C, Seiwert T Y, Vora S, Kanteti R, Hasina R, Carey G B, Surati M, Weichselbaum R R, Lingen M W, Vokes E E, Salgia R. Expression and mutational analysis of c-CBL and its relationship to the MET receptor in head and neck squamous cell carcinoma. Oncotarget. 2017 Mar. 21; 8 (12): 18726-18734.
Scagliotti G, Moro-Sibilot D, Kollmeier J, Favaretto A, Cho E K, Grosch H, Kimmich M, Girard N, Tsai C M, Hsia T C, Brighenti M, Schumann C, Wang X A, Wijayawardana S R, Gruver A M, Wallin J, Mansouri K, Wacheck V, Chang G C. A Randomized-Controlled Phase 2 Study of the MET Antibody Emibetuzumab in Combination with Erlotinib as First-Line Treatment for EGFR Mutation-Positive NSCLC Patients. J Thorac Oncol. 2020 January; 15 (1): 80-90.
Seiwert T, Sarantopoulos J, Kallender H, McCallum S, Keer H N, Blumenschein G Jr. Phase II trial of single-agent foretinib (GSK1363089) in patients with recurrent or metastatic squamous cell carcinoma of the head and neck. Invest New Drugs. 2013 April; 31 (2): 417-24.
Seiwert T Y, Jagadeeswaran R, Faoro L, Janamanchi V, Nallasura V, El Dinali M, Yala S, Kanteti R, Cohen E E, Lingen M W, Martin L, Krishnaswamy S, Klein-Szanto A, Christensen J G, Vokes E E, Salgia R. The MET receptor tyrosine kinase is a potential novel therapeutic target for head and neck squamous cell carcinoma. Cancer Res. 2009 Apr. 1; 69 (7): 3021-31.
Sen B, Peng S, Saigal B, Williams M D, Johnson F M. Distinct interactions between c-Src and c-Met in mediating resistance to c-Src inhibition in head and neck cancer. Clin Cancer Res. 2011 Feb. 1; 17 (3): 514-24.
SenthilKumar G, Fisher M M, Skiba J H, Miller M C, Brennan S R, Kaushik S, Bradley S T, Longhurst C A, Buehler D, Nickel K P, Iyer G, Kimple R J, Baschnagel A M. FGFR Inhibition Enhances Sensitivity to Radiation in Non-Small Cell Lung Cancer. Mol Cancer Ther. 2020 June; 19 (6): 1255-1265.
Spigel D R, Edelman M J, O'Byrne K, Paz-Ares L, Mocci S, Phan S, Shames D S, Smith D, Yu W, Paton V E, Mok T. Results From the Phase III Randomized Trial of Onartuzumab Plus Erlotinib Versus Erlotinib in Previously Treated Stage IIIB or IV Non-Small-Cell Lung Cancer: METLung. J Clin Oncol. 2017 February; 35 (4): 412-420.
Spigel D R, Ervin T J, Ramlau R A, Daniel D B, Goldschmidt J H Jr, Blumenschein G R Jr, Krzakowski M J, Robinet G, Godbert B, Barlesi F, Govindan R, Patel T, Orlov S V, Wertheim M S, Yu W, Zha J, Yauch R L, Patel P H, Phan S C, Peterson A C. Randomized phase II trial of Onartuzumab in combination with erlotinib in patients with advanced non-small-cell lung cancer. J Clin Oncol. 2013 Nov. 10; 31 (32): 4105-14.
Stabile L P, He G, Lui V W, Thomas S, Henry C, Gubish C T, Joyce S, Quesnelle K M, Siegfried J M, Grandis J R. c-Src activation mediates erlotinib resistance in head and neck cancer by stimulating c-Met. Clin Cancer Res. 2013 Jan. 15; 19 (2): 380-92. doi: 10.1158/1078-0432.CCR-12-1555. Epub 2012 Dec. 4. Erratum in: Clin Cancer Res. 2013 Jul. 1; 19 (13): 3715.
Strickler J H, LoRusso P, Salgia R, Kang Y K, Yen C J, Lin C C, Ansell P, Motwani M, Wong S, Yue H, Wang L, Reilly E, Afar D, Naumovski L, Ramanathan R K. Phase I Dose-Escalation and -Expansion Study of Telisotuzumab (ABT-700), an Anti-c-Met Antibody, in Patients with Advanced Solid Tumors. Mol Cancer Ther. 2020 May; 19 (5): 1210-1217.
Su Z, Han Y, Sun Q, Wang X, Xu T, Xie W, Huang X. Anti-MET VHH Pool Overcomes MET-Targeted Cancer Therapeutic Resistance. Mol Cancer Ther. 2019 January; 18 (1): 100-111.
Szturz P, Budíková M, Vermorken J B, Horová I, Gál B, Raymond E, de Gramont A, Faivre S. Prognostic value of c-MET in head and neck cancer: A systematic review and meta-analysis of aggregate data. Oral Oncol. 2017 November; 74:68-76.
Yücel O T, Sungur A, Kaya S. c-met overexpression in supraglottic laryngeal squamous cell carcinoma and its relation to lymph node metastases. Otolaryngol Head Neck Surg. 2004 June; 130 (6): 698-703.
Trusolino L, Bertotti A, Comoglio P M. MET signalling: principles and functions in development, organ regeneration and cancer. Nat Rev Mol Cell Biol. 2010 December; 11 (12): 834-48.
Van Audenhove I, Gettemans J. Nanobodies as Versatile Tools to Understand, Diagnose, Visualize and Treat Cancer. EBioMedicine. 2016 June; 8:40-48.
Vsiansky V, Gumulec J, Raudenska M, Masarik M. Prognostic role of c-Met in head and neck squamous cell cancer tissues: a meta-analysis. Sci Rep. 2018 Jul. 10; 8 (1): 10370.
Wang L, Tang G, Hu K, Liu X, Zhou W, Li H, Huang S, Han Y, Chen L, Zhong J, Wu H. Comparison of 68Ga-FAPI and 18F-FDG PET/C T in the Evaluation of Advanced Lung Cancer. Radiology. 2022 April; 303 (1): 191-199.
Waqar S N, Redman M W, Arnold S M, Hirsch F R, Mack P C, Schwartz L H, Gandara D R, Stinchcombe T E, Leighl N B, Ramalingam S S, Tanna S H, Raddin R S, Minichiello K, Bradley J D, Kelly K, Herbst R S, Papadimitrakopoulou V A. A Phase II Study of Telisotuzumab Vedotin in Patients With c-MET-positive Stage IV or Recurrent Squamous Cell Lung Cancer (LUNG-MAP Sub-study S1400K, NCT03574753). Clin Lung Cancer. 2021 May; 22 (3): 170-177.
Xi W H, Yang L Y, Cao Z Y, Qian Y. Tivantinib (ARQ-197) exhibits anti-tumor activity with down-regulation of FAK in oral squamous cell carcinoma. Biochem Biophys Res Commun. 2015 Feb. 20; 457 (4): 723-9.
Xu H, Stabile L P, Gubish C T, Gooding W E, Grandis J R, Siegfried J M. Dual blockade of EGFR and c-Met abrogates redundant signaling and proliferation in head and neck carcinoma cells. Clin Cancer Res. 2011 Jul. 1; 17 (13): 4425-38.
Ye G, Gallant J, Zheng J, Massey C, Shi K, Tai W, Odle A, Vickers M, Shang J, Wan Y, Du L, Aihara H, Perlman S, LeBeau A, Li F. The development of Nanosota-1 as anti-SARS-CoV-2 nanobody drug candidates. Elife. 2021 Aug. 2; 10: e64815.
Yi C A, Shin K M, Lee K S, Kim B T, Kim H, Kwon O J, Choi J Y, Chung M J. Non-small cell lung cancer staging: efficacy comparison of integrated PET/C T versus 3.0-T whole-body M R imaging. Radiology. 2008 August; 248 (2): 632-42.
Zavoiura O, Brunner B, Casteels P, Zimmermann L, Ozog M, Boutton C, Helms M W, Wagenaar T, Adam V, Peterka J, Metz-Weidmann C, Deschaght P, Scheidler S, Jahn-Hofmann K. Nanobody-siRNA Conjugates for Targeted Delivery of siRNA to Cancer Cells. Mol Pharm. 2021 Mar. 1; 18 (3): 1048-1060.
Zeglis B M, Lewis J S. The bioconjugation and radiosynthesis of 89Zr-DFO-labeled antibodies. J Vis Exp. 2015 Feb. 12; (96): 52521.
Zhang Y W, Wang L M, Jove R, Vande Woude G F. Requirement of Stat3 signaling for HGF/S F-Met mediated tumorigenesis. Oncogene. 2002 Jan. 10; 21 (2): 217-26.

What is claimed is:

1. An isolated anti-MET construct comprising a single-domain antibody (sdAb) moiety specifically recognizing MET, wherein the sdAb moiety comprises a CDR1, a CDR2, and a CDR3, wherein:
the CDR1 comprises the amino acid sequence of GFX¹FX²X³YDMX⁴ (SEQ ID NO:1), wherein:
X¹ is N or T;
X² is E or G;
X³ is R or A; and
X⁴ is S or V,
or a variant thereof comprising up to 3 (such as any of 1, 2, or 3) amino acid substitutions;
the CDR2 comprises the amino acid sequence of X⁵X⁶X⁷X⁸X⁹GX¹⁰X¹¹X¹²X¹³, wherein:
X⁵ is R or F;
X⁶ is L or I;
X⁷ is N or S;
X⁸ is S or N;
X⁹ is F or G;
X¹⁰ is R or E;
X¹¹ is S or E;
X¹² is T or V; and
X¹³ is Y or S,
or a variant thereof comprising up to 3 (such as any of 1, 2, or 3) amino acid substitutions; and/or
the CDR3 comprises the amino acid sequence of RSTDVSPGLSSWWTYEYDV (SEQ ID NO: 11), or a variant thereof comprising up to 3 (such as any of 1, 2, or 3) amino acid substitutions.

2. The isolated anti-MET construct of claim 1, wherein:
the CDR1 comprises the amino acid sequence of any one of GFNFERYDMS (SEQ ID NO: 7) and GFTFGAYDMV (SEQ ID NO:8), or a variant thereof comprising up to 3 (such as any of 1, 2, or 3) amino acid substitutions;
the CDR2 comprises the amino acid sequence of any one of RLNSFGRSTY (SEQ ID NO: 9) and FISNGGEEVS (SEQ ID NO:10), or a variant thereof comprising up to 3 (such as any of 1, 2, or 3) amino acid substitutions; and/or
the CDR3 comprises the amino acid sequence of RSTDVSPGLSSWWTYEYDV (SEQ ID NO: 11), or a variant thereof comprising up to 3 (such as any of 1, 2, or 3) amino acid substitutions.

3. The isolated anti-MET construct of claim 1, wherein:
the CDR1 comprises the amino acid sequence of any one of GENFERYDMS (SEQ ID NO: 7) and GFTFGAYDMV (SEQ ID NO:8);
the CDR2 comprises the amino acid sequence of any one of RLNSFGRSTY (SEQ ID NO: 9) and FISNGGEEVS (SEQ ID NO:10); and/or
the CDR3 comprises the amino acid sequence of RSTDVSPGLSSWWTYEYDV (SEQ ID NO: 11).

4. The isolated anti-MET construct of claim 1, wherein:
the CDR1 comprises the amino acid sequence of GENFERYDMS (SEQ ID NO:7) or a variant thereof comprising up to 3 (such as any of 1, 2, or 3) amino acid substitutions, a CDR2 comprising the amino acid sequence of RLNSFGRSTY (SEQ ID NO:9) or a variant thereof comprising up to 3 (such as any of 1, 2, or 3) amino acid substitutions, and a CDR3 comprising the amino acid sequence of RSTDVSPGLSSWWTYEYDV (SEQ ID NO:11) or a variant thereof comprising up to 3 (such as any of 1, 2, or 3) amino acid substitutions; or
the CDR1 comprises the amino acid sequence of GFTFGAYDMV (SEQ ID NO:8) or a variant thereof comprising up to 3 (such as any of 1, 2, or 3) amino acid substitutions, a CDR2 comprising the amino acid sequence of FISNGGEEVS (SEQ ID NO:10) or a variant thereof comprising up to 3 (such as any of 1, 2, or 3) amino acid substitutions, and a CDR3 comprising the amino acid sequence of RSTDVSPGLSSWWTYEYDV (SEQ ID NO:11) or a variant thereof comprising up to 3 (such as any of 1, 2, or 3) amino acid substitutions.

5. The isolated anti-MET construct of claim 1, wherein the sdAb moiety comprises:
the CDR1 comprises the amino acid sequence of GENFERYDMS (SEQ ID NO:7), a CDR2 comprising the amino acid sequence of RLNSFGRSTY (SEQ ID NO:9), and a CDR3 comprising the amino acid sequence of RSTDVSPGLSSWWTYEYDV (SEQ ID NO:11); or
the CDR1 comprises the amino acid sequence of GFTFGAYDMV (SEQ ID NO:8), a CDR2 comprising the amino acid sequence of FISNGGEEVS (SEQ ID NO:10), and a CDR3 comprising the amino acid sequence of RSTDVSPGLSSWWTYEYDV (SEQ ID NO:11).

6. The isolated anti-MET construct of claim 1, wherein the sdAb moiety comprises:
an FR1 comprising the amino acid sequence of EX¹⁴QLVESGGX¹⁵LVQPGGSLRLSCX¹⁶AS (SEQ ID NO:3), wherein:
X¹⁴ is V or A;
X¹⁵ is G or A; and
X¹⁶ is E or A,
or a variant thereof comprising up to 3 (such as any of 1, 2, or 3) amino acid substitutions;
an FR2 comprising the amino acid sequence of WVRX¹⁷AX¹⁸GKGPEWX¹⁹S (SEQ ID NO: 4), wherein:
X¹⁷ is Q or H;
X¹⁸ is P or T; and
X¹⁹ is V or I,
or a variant thereof comprising up to 3 (such as any of 1, 2, or 3) amino acid substitutions;
an FR3 comprising the amino acid sequence of YX²⁰X²¹SVKX²²RFTX²³SRDX²⁴AX²⁵X²⁶X²⁷X²⁸YLQMNNLKPEDX²⁹X³⁰VYYCA (SEQ ID NO: 5), wherein:
X²⁰ is L or A;
X²¹ is D or S;
X²² is G or D;
X²³ is V or I;
X²⁴ is N or T;
X²⁵ is Q or K;
X²⁶ is N or S;
X²⁷ is M or T;
X²⁸ is L or F;
X²⁹ is T or A; and
X³⁰ is A or G,
or a variant thereof comprising up to 3 (such as any of 1, 2, or 3) amino acid substitutions; and/or
an FR4 comprising the amino acid sequence of WGQGTX³¹VTVSS (SEQ ID NO:6), wherein X³¹ is Q or L, or a variant thereof comprising up to 3 (such as any of 1, 2, or 3) amino acid substitutions.

7. The isolated anti-MET construct of claim 1, wherein the sdAb moiety comprises a VHH domain comprising the CDR1, the CDR2, and the CDR3, wherein:
the VHH domain comprises the amino acid sequence of SEQ ID NO:22 or a variant thereof having at least 90% or at least 95% sequence identity to SEQ ID NO:22 wherein the CDR1 comprises the amino acid sequence of GENFERYDMS (SEQ ID NO:7), the CDR2 comprises the amino acid sequence of RLNSFGRSTY (SEQ ID NO:9), and the CDR3 comprises the amino acid sequence of RSTDVSPGLSSWWTYEYDV (SEQ ID NO:11);
the VHH domain comprising the amino acid sequence of SEQ ID NO:24 or a variant thereof having at least 90% or at least 95% sequence identity to SEQ ID NO:24 wherein the CDR1 comprises the amino acid sequence of GENFERYDMS (SEQ ID NO:7), the CDR2 comprises the amino acid sequence of RLNSFGRSTY (SEQ ID NO:9), and the CDR3 comprises the amino acid sequence of RSTDVSPGLSSWWTYEYDV (SEQ ID NO:11); or
the VHH domain comprising the amino acid sequence of SEQ ID NO:26, or a variant thereof having at least 90% or at least 95% sequence identity to SEQ ID NO:26 wherein the CDR1 comprises the amino acid sequence of GFTFGAYDMV (SEQ ID NO:8), the CDR2 comprises the amino acid sequence of FISNGGEEVS (SEQ ID NO:10), and the CDR3 comprises the amino acid sequence of RSTDVSPGLSSWWTYEYDV (SEQ ID NO:11).

8. The isolated anti-MET construct of claim 1, wherein the sdAb moiety specifically recognizing MET is camelid, chimeric, partially humanized, or fully humanized.

9. The isolated anti-MET construct of claim 1, wherein the sdAb moiety specifically recognizing MET is fused to a human IgG1 Fc.

10. The isolated anti-MET construct of claim 1, wherein the isolated anti-MET construct is a heavy chain-only antibody.

11. The isolated anti-MET construct of claim 1, wherein the isolated anti-MET construct is fused to a second antibody moiety.

12. The isolated anti-MET construct of claim 1, wherein the isolated anti-MET construct is fused to an anti-EGFR antibody moiety.

13. The isolated anti-MET construct of claim 1, wherein the isolated anti-MET construct is labeled.

14. The isolated anti-MET construct of claim 1, wherein the isolated anti-MET construct is radiolabeled.

15. The isolated anti-MET construct of claim 1, wherein the sdAb moiety is conjugated to a cytotoxic agent.

16. A method of treating a MET-related condition in an individual in need thereof, comprising administering to the individual a therapeutically effective amount of the isolated anti-MET construct of claim 1.

17. The method of claim 16, wherein the MET-related condition is cancer.

18. The method of claim 17, wherein the cancer is selected from the group consisting of lung cancer and head and neck cancer.

19. The method of claim 16, further comprising screening the individual, comprising administering a screening amount of the isolated anti-MET construct to the individual and imaging the individual for presence of the isolated anti-MET construct in the individual.

20. A method of screening an individual, comprising administering a screening amount of the isolated anti-MET construct of claim 1 to the individual and imaging the individual for presence of the isolated anti-MET construct in the individual.
